(19) United States
(12) Patent Application Publication
(10) Pub. No.: US 2019/0082968 A1
(43) Pub. Date: Mar. 21, 2019

(54) SYSTEM AND METHOD OF CONTINUOUS HEALTH MONITORING

(71) Applicants: Prasad Karnik, Ladera Ranch, CA (US); Sumukh Pathare, Ladera Ranch, CA (US)

(72) Inventors: Prasad Karnik, Ladera Ranch, CA (US); Sumukh Pathare, Ladera Ranch, CA (US)

(21) Appl. No.: 16/133,309

(22) Filed: Sep. 17, 2018

Related U.S. Application Data
(60) Provisional application No. 62/558,995, filed on Sep. 15, 2017.

(51) Int. Cl. A61B 5/01; A61B 5/00; G16H 80/00; G06F 3/0482

(52) U.S. Cl. CPC A61B 5/01; A61B 5/0008; G16H 80/00; G06F 3/0482; A61B 2562/166; A61B 5/6831; A61B 5/6833; A61B 5/6804; A61B 2562/0271; A61B 5/4306

(57) ABSTRACT

The invention involves a system and method implementing a wearable device that employs a flexible printed circuit board (PCB), which includes a temperature sensor. The PCB may be printed on a flexible substrate that may be folded to form multiple layers configured to house the sensor and an antenna. The sensor may be housed within said layers and situated at a terminal end of a pathway that may be printed on the PCB, wherein the pathway acts as a contact as well as a conduit of heat from the body of the user to the sensor. The antenna may be housed within the layers of the flexible PCB in a manner such that proper signal transmission is preserved and latency is minimized. Temperature readings may be wirelessly communicated to one or more client devices, which implement one or more algorithms suitable for generating insights regarding health aspects of the user.

### PRIORITY NOTICE

The present application claims priority under 35 U.S.C. § 119 to U.S. Provisional Application 62/558,995, filed Sep. 15, 2017, the disclosure of which is incorporated herein by reference in its entirety.

### TECHNICAL FIELD OF THE INVENTION

The present invention relates in general to a system and method of continuous health monitoring, and more specifically, to continuous health monitoring using a wearable device that wirelessly communicates electrical activity, which may be in conjunction with improved temperature readings, to one or more client devices.

### COPYRIGHT AND TRADEMARK NOTICE

A portion of the disclosure of this patent application may contain material that is subject to copyright protection. The owner has no objection to the facsimile reproduction by anyone of the patent document or the patent disclosure, as it appears in the Patent and Trademark Office patent file or records, but otherwise reserves all copyrights whatsoever.

Certain marks referenced herein may be common law or registered trademarks of third parties affiliated or unaffiliated with the applicant or the assignee. Use of these marks is by way of example and should not be construed as descriptive or to limit the scope of this invention to material associated only with such marks.

### BACKGROUND OF THE INVENTION

Health monitoring of individuals using wearable devices is becoming a widespread practice. The prior art is beginning to see more and more wearables that can monitor and track different metrics such as how many steps a user takes during the day, what their approximate heart rate is at a given time, etc. Other metrics are more difficult to read with popular devices such as the smart watches and fitness bands of today. For example, many smart watches and fitness bands fail to include hardware that reads certain critical vital signs—like body temperature.

Reading the body's core temperature accurately is not easily achieved by placing a sensor in just any area of the human body; for example, a smart watch equipped with a temperature sensor for example is unlikely to properly read a user's body core temperature because the wrist is not a medically recognized region for accurately taking temperature measurements—accordingly, any reading of temperature may simply be undependable with common wearables.

However, there have been some developments in the continuous monitoring of the body's core temperature; several wireless solutions exist that consist of a wearable device configured to monitor the temperature of a user, which can connect to a smartphone or tablet for reception of temperature data. Some of these newer devices attempt to monitor skin temperature in the armpit or axillary region where there is medical acceptance. Yet, these devices are riddled with shortcomings.

One persistent problem in body-worn wearable devices is the design and placement of antennas. For example, some devices, designed to be placed at the axillary region, fail in function because the axillary region is an enclosed cavity surrounded by body tissue that severely attenuates radio frequency signal, thereby rendering the device unable to properly transmit the temperature measured to a listening device. Thus, it is desirable to have a device with an appropriately designed antenna suitable for overcoming signal transmission challenges related to the human anatomy.

Another problem is the delay in getting an accurate measurement of body temperature after applying the device on the body. Any measurement means that uses a temperature sensor suffers latency that comes from having to get the temperature sensor in thermal equilibrium with the body—usually causing a delay in the order of minutes. This delay is not acceptable to users who are looking for an accurate reading in seconds. Part of the problem resides in the material and means used for conducting heat. The devices disclosed in U.S. Patent Application 2016/0183794 to Gannon et al, for example, suffers from the shortcomings mentioned above. Thus, it is desirable to minimize the latency in a continuous monitoring device so that information may be gleaned quickly and acted upon as needed.

Yet another problem is providing proper battery life suitable for effective continuous health care monitoring; battery life is a challenge for wearable devices and has not been adequately addressed. Many wearable devices now use Bluetooth Low-Energy (BLE) to lower the power consumed over the communications link. The power consumed by these devices is still too high to allow wearable devices to last for many months without recharging or without using a large and intrusive primary battery. For a wearable device that is meant to be used for continuous monitoring, recharging is a problem because the time taken for recharging keeps the device from being used for monitoring. The problem gets worse for a device that is designed to monitor and act continuously such as in delivering a continuous dose of medication; these devices can't be allowed to take time off for recharging. The ideal solution is a device that can be used without recharging for as long as possible, wherein the lifetime of the product becomes its differentiating feature.

One factor in power consumption is the power consumed by communication of data from the wearable device. US Patent Application 2016/0095047 to Lee et al. and U.S. Pat. No. 8,208,973 to Mehta address the problem by implementation of various time intervals for multiple different advertising packets or in different operating modes. However, as will be explained below, certain battery parameters are not fully factored by the methods in these disclosures, which may be useful in determination of time intervals for advertising packets or the contents of the packets so as to further increase power efficiency and generally prolong battery life of wearable devices.

Yet another problem is keeping data provided by these devices adequately secure. A medical device should be designed to provide privacy of patient data over communication and storage. Typically, wireless medical devices transfer patient data to a single device over a dedicated connection, or broadcast patient data to any device that is within listening range. The former approach restricts the use of the monitoring solution to only one user, which can be a single point of failure when the monitoring device is out of range or powered down. The latter approach of data broadcast is more reliable as the broadcasts can be received by several monitoring devices that are in range; but as the data is transmitted on the broadcast channel, patient data is no longer private. To ensure secure transmission over a wireless communication system, patient data requires additional encryption not provided by transmission protocols over a broadcast channel. To encrypt patient data, encryption keys have to be shared between the medical device and the device receiving the patient data, and it is known that these encryption keys may be intercepted. U.S. Pat. No. 8,130,958 to Schrum and US Patent Application 2002/0123325A1 to Cooper address the problem by reducing transmission power so that proximity is required between the two devices that are communicating with each other. The limitations of these two approaches is that even at lowest power settings, a receiver dedicated to eavesdropping on a channel can do so from several feet. Thus, it is necessary for a medical device to protect user data from unauthorized access by storing and transmitting data securely; although several solutions have been disclosed, the problem has not been adequately addressed.

Yet another problem that has not been adequately addressed is providing timely insights and providing these insights via meaningful alerts to the right entities. A key aspect of health monitoring is alerting to detected changes in the condition of the user and the condition of the monitoring device. Certain insights need to be communicated to the user or an authorized person as soon as possible. An example may include detection of low-grade fever, which typically only the user or the caregiver may need to be made aware. However, when fever in cancer patients exceeds a certain threshold, it is often necessary to have the patient's clinic or emergency room be advised of the developing condition. Thus, it is desirable that a system of a wearable device generate proper insights and implement adequate notifications.

Yet another problem faced by wearables is the device's adaptability to the wearer's environment. Wearable devices for continuous monitoring may be worn all the time, but there are times when these devices should be put in a non-transmitting mode even when worn. For example, this is true when users are passing through security checks or when they are flying on an airplane. Thus, it is desirable that a wearable device be made adaptable to different environmental conditions.

Therefore, there exists a previously unappreciated need for a new and improved system and method of continuous health monitoring that: is suitable for overcoming certain signal transmission challenges; minimizes latency so that information may be gleaned and acted upon quickly as needed; increases efficiency and generally prolongs battery life of the wearable device; protects user data from unauthorized access by storing and transmitting data securely; may be easily adapted for different environmental conditions; and is configured to generate proper insights and implement adequate notifications concerning the user.

It is to these ends that the present invention has been developed.

### SUMMARY OF THE INVENTION

To minimize the limitations in the prior art, and to minimize other limitations that will be apparent upon reading and understanding the present specification, the present invention describes a system and method of continuous health monitoring, which implements a wearable device that wirelessly communicates improved temperature readings to one or more client devices.

Generally, the invention involves a system comprising at least one wearable device that employs a flexible printed circuit board (PCB), which includes a temperature sensor. The flexible PCB may be printed on a flexible substrate that may be folded to form multiple layers configured to house the sensor and an antenna. The sensor may be housed within said layers and situated at a terminal end of a pathway that may be printed on the PCB, which conducts heat from the body of the user to the sensor. The antenna may be housed within the layers of the flexible PCB in a manner such that proper signal transmission is preserved, and latency is minimized. A part of the flexible PCB may be folded around a battery to connect to the terminals of the battery. Temperature readings may be wirelessly communicated to one or more client devices, which implement one or more algorithms suitable for generating insights regarding one or more health aspects of the user. The system may also further comprise another wearable device suitable for reading multiple voltage differentials throughout the human body of a user. In exemplary embodiments, such wearable device may comprise a plurality of contacts, some of which may be applied directly to the body and some of which may be disposed on a surface of the device for a user to interact with. A multiplexer in communication with a microprocessor housed within the wearable device may be used to receive and process the voltage differences; a communications module may transmit the voltage differentials to one or more client devices. As may be appreciated, these signals pertaining to voltage differentials throughout the body may be utilized to detect abnormalities indicative of a health condition, and or used for monitoring the health of the user. In certain embodiments, the voltage differentials may be used to measure the electrocardiogram (ECG or EKG) of the user so as to determine the cardiac health of the user, the detection of which can lead to timely intervention for ischemia, fibrillation, arrhythmia, tachycardia or any other heart condition that is detectable using ECG. In certain other embodiments, the voltage differentials may be used to measure neural activity to detect the onset of seizures and the detection of epilepsy, and to provide feedback on an autistic user's ability to learn. In exemplary embodiments, a feedback module may be incorporated such as a haptic device to alert the user in the event an anomaly in the voltage differential is detected. As may be appreciated by a person of skill in the medical field, such wearable device has many applications, including but not limited to, monitoring a cardiovascular health of the user. In some exemplary embodiments, one or more of the wearable devices communicate user data to a client device such as a smartphone. In some exemplary embodiments, one or more of the wearable devices further communicate data to a server. In some exemplary embodiments, the server may host a user interface such as a website or mobile application so that one or more users may access the user data remotely.

Moreover, several methods may be implemented to: determine body core temperature; determine basal body temperature; measure, communicate and alert of physical parameters of human body such as axillary temperature to monitor fever or to detect the basal body temperature that forms the basis for ovulation monitoring or hormonal deficiency; provide one or more insights concerning health aspects of a user; determine a wearable device state; prolong battery life; and maintain secured user data for patient privacy and reliability of communication of said data.

A wearable device, in accordance with an exemplary embodiment of the present invention, comprises: a flexible printed circuit board (PCB) that may be folded to form multiple layers; a temperature sensor situated on a first surface of a first layer of the PCB; a circuit including a copper contact region etched on a second surface of the first layer of the PCB, the copper circuit including one or more copper pathways connecting the thermal contact region of the copper circuit to the temperature sensor; a communication transmitter including an antenna situated on one of the multiple layers of the PCB; and a microprocessor in communication with the temperature sensor and the communication transmitter, the microprocessor configured to continuously obtain temperature sensing data from the temperature sensor and transmit the sensing data to one or more client devices.

A system for continuous health monitoring, in accordance with an exemplary embodiment of the present invention, comprises: a server for storing health data including temperature data; a wearable device including: a flexible printed circuit board (PCB) that is folded to form multiple layers; a temperature sensor situated on a first surface of a first layer of the PCB; a circuit including a thermal contact region etched on a second surface of the first layer of the PCB, the circuit including one or more pathways connecting the contact region of the circuit to the temperature sensor; a communication transmitter including an antenna situated on one of the multiple layers of the PCB; and a microprocessor in communication with the temperature sensor and the communication transmitter, the microprocessor configured to: continuously obtain temperature sensing data from the temperature sensor; and transmit the sensing data to one or more client devices; and a user interface executable by one of the one or more client devices configured to display information associated with the temperature sensing data.

A method in accordance with practice of an exemplary embodiment of the present invention, comprises: A method of continuous health monitoring implemented by a wearable device, comprising: receiving temperature data from one or more sensors on a flexible printed circuit board (PCB) that is folded to form multiple layers, wherein the one or more temperature sensors are situated on a first surface of a first layer of the PCB, including a copper circuit for transferring heat from a copper thermal contact etched on a second surface of the first layer of the PCB, the copper circuit including one or more copper pathways connecting the copper contact of the copper circuit to the temperature sensor; generating one or more data packets associated with the temperature data; and sending via a communication transmitter including an antenna situated on one of the multiple layers of the PCB, the one or more data packets associated with the temperature data to a client device.

Various objects and advantages of the present invention will become apparent from the following description taken in conjunction with the accompanying drawings wherein are set forth, by way of illustration and example, certain embodiments of this invention. The drawings submitted herewith constitute a part of this specification, include exemplary embodiments of the present invention, and illustrate various objects and features thereof.

### BRIEF DESCRIPTION OF THE DRAWINGS

Elements in the figures have not necessarily been drawn to scale in order to enhance their clarity and improve understanding of the various embodiments of the invention. Furthermore, elements that are known to be common and well understood to those in the industry are not depicted in order to provide a clear view of the various embodiments of the invention. The drawings that accompany the detailed description can be briefly described as follows:

FIG. 1(a) illustrates a system in accordance with an exemplary embodiment of the present invention.

FIG. 1(b) illustrates a diagram of a wearable device in accordance with an exemplary embodiment of the present invention.

FIG. 2(a) depicts a bottom view of a flexible PCB for a wearable device in accordance with an exemplary embodiment of the present invention.

FIG. 2(b) depicts a top view of a flexible PCB for a wearable device in accordance with an exemplary embodiment of the present invention.

FIG. 2(c) depicts a flexible PCB partially coupled to a plastic layer in accordance with an exemplary embodiment of the present invention.

FIG. 2(d) depicts a top view of the flexible PCB partially folded over the foam layer depicted in FIG. 2(c).

FIG. 2(e) depicts a bottom view of the flexible PCB partially folded over and threaded through a portion of the foam layer depicted in FIG. 2(a).

FIG. 3(a) depicts a bottom view of a flexible PCB for a wearable device in accordance with an exemplary embodiment of the present invention.

FIG. 3(b) depicts a top view of a flexible PCB for a wearable device in accordance with an exemplary embodiment of the present invention.

FIG. 3(c) depicts an exploded view of the flexible PCB depicted in FIG. 3(a)-(b), including a housing in accordance with an exemplary embodiment of the present invention.

FIG. 3(d) depicts a top view of the flexible PCB depicted in FIG. 3(a)-(b), placed inside a first portion of the housing depicted in FIG. 3(c).

FIG. 3(e) depicts a bottom view of the flexible PCB depicted in FIG. 3(a)-(b), placed inside a second portion of the housing depicted in FIG. 3(c).

FIG. 3(f)-3(h) depict the wearable device fully enclosed in the housing depicted in FIG. 3(c).

FIG. 3(i) depicts an exploded view of the flexible PCB depicted in FIG. 3(a)-(b), including a housing in accordance with an exemplary embodiment of the present invention.

FIG. 3(j) depicts a top view of the flexible PCB depicted in FIG. 3(a)-(b), placed inside a first portion of the housing depicted in FIG. 3(i).

FIG. 3(k) depicts a bottom view of the flexible PCB depicted in FIG. 3(a)-(b), placed inside a second portion of the housing depicted in FIG. 3(i).

FIG. 4(a) illustrates different folds of the PCB within layers of the device in accordance with an exemplary embodiment of the present invention.

FIG. 4(b) illustrates a side view of a folded PCB and different layers formed therein in accordance with an exemplary embodiment of the present invention.

FIG. 4(c) illustrates a sensor and heat pathway of an exemplary PCB in accordance with the present invention.

FIG. 4(d) illustrates a close-up view of an exemplary embodiment of a flexible PCB and foam configuration in accordance with the present invention.

FIG. 5(a) illustrates a flow chart of an exemplary method of reading electrical activity, which may be in conjunction with temperature data in accordance an exemplary embodiment of the present invention.

FIG. 5(b) illustrates a block diagram of a wearable device including multiple electrically-conducting contacts and does not necessarily include a temperature sensor, this wearable device configured to generate voltage differentials pertaining to a user, in accordance with exemplary embodiments of the present invention.

FIG. 5(c) and FIG. 5(d) illustrate an enclosure or housing for the exemplary wearable device depicted in the block diagram of FIG. 5(b).

FIG. 5(e) is a flow chart depicting an exemplary method employed by an exemplary embodiment of the wearable device illustrated in FIG. 5(b)-FIG. 5(c).

FIG. 6 is a flow chart depicting an exemplary method of deriving basal body temperature in exemplary practice of an embodiment of the present invention.

FIG. 7(a) is a chart showing temperature as a function of time, which demonstrates a method for determining different device states in exemplary practice of an embodiment of the present invention.

FIG. 7(b) is a flow chart depicting an exemplary method of deriving and presenting insights to a user in exemplary practice of an embodiment of the present invention.

FIG. 7(c) is a chart showing temperature as a function of time, which demonstrates a method of deriving useful insights in exemplary practice of an embodiment of the present invention.

FIG. 8 is a flow chart depicting an exemplary method of throttling data in exemplary practice of an embodiment of the present invention.

FIG. 9 is a flow chart depicting an exemplary processing flow of typical programmable instructions on a wearable device in accordance with the present invention.

FIG. 10 is a flow chart depicting an exemplary method of communicating user data between a wearable device and a client device in exemplary practice of an embodiment of the present invention.

FIG. 11 is a flow chart depicting an exemplary method of authorizing one or more devices to read data captured by a wearable device in exemplary practice of an embodiment of the present invention.

FIG. 12 is a flow chart depicting an exemplary method of authorizing one or more devices to read data captured by a wearable device in exemplary practice of an embodiment of the present invention.

FIG. 13-FIG. 29 illustrate an exemplary user interface that may be distributed to a plurality of client devices in accordance with exemplary embodiments of the present invention.

### DETAILED DESCRIPTION OF THE INVENTION

In the following discussion that addresses a number of embodiments and applications of the present invention, reference is made to the accompanying drawings that form a part thereof, where depictions are made, by way of illustration, of specific embodiments in which the invention may be practiced. It is to be understood that other embodiments may be utilized and changes may be made without departing from the scope of the invention. Wherever possible, the same reference numbers are used in the drawings and the following description to refer to the same or similar elements.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment.

The terms “comprising,” “including,” “having,” and the like are synonymous and are used inclusively, in an open-ended fashion, and do not exclude additional elements, features, acts, operations and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require at least one of X, at least one of Y, and at least one of Z to each be present. The term “and/or” means that “and” applies to some embodiments and “or” applies to some embodiments. Thus, A, B, and/or C can be replaced with A, B, and C written in one sentence and A, B, or C written in another sentence. A, B, and/or C means that some embodiments can include A and B, some embodiments can include A and C, some embodiments can include B and C, some embodiments can only include A, some embodiments can include only B, some embodiments can include only C, and some embodiments include A, B, and C. The term “and/or” is used to avoid unnecessary redundancy.

While embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Thus, nothing in the foregoing description is intended to imply that any particular feature, characteristic, step, module, or block is necessary or indispensable. Indeed, the novel methods and systems described herein may be embodied in a variety of other forms; furthermore, various omissions, substitutions, and changes in the form of the methods and systems described herein may be made without departing from the spirit of the inventions disclosed herein. Accordingly, the following detailed description does not limit the disclosure. Instead, the proper scope of the disclosure is defined by the appended claims.

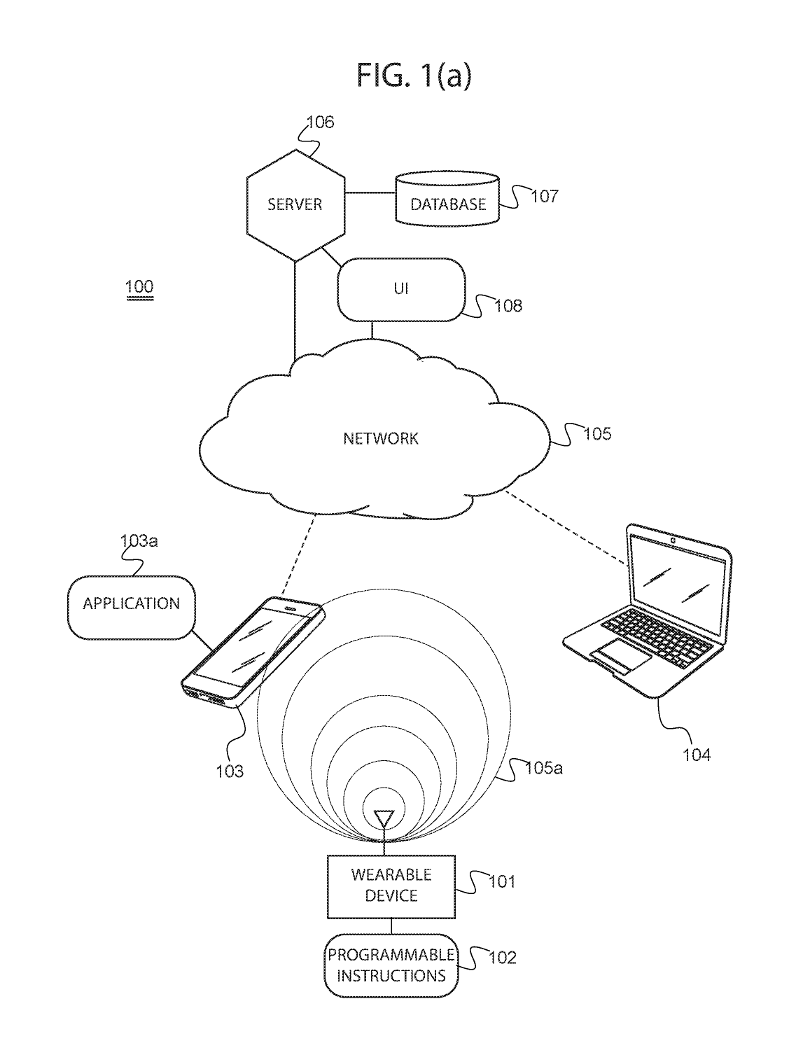

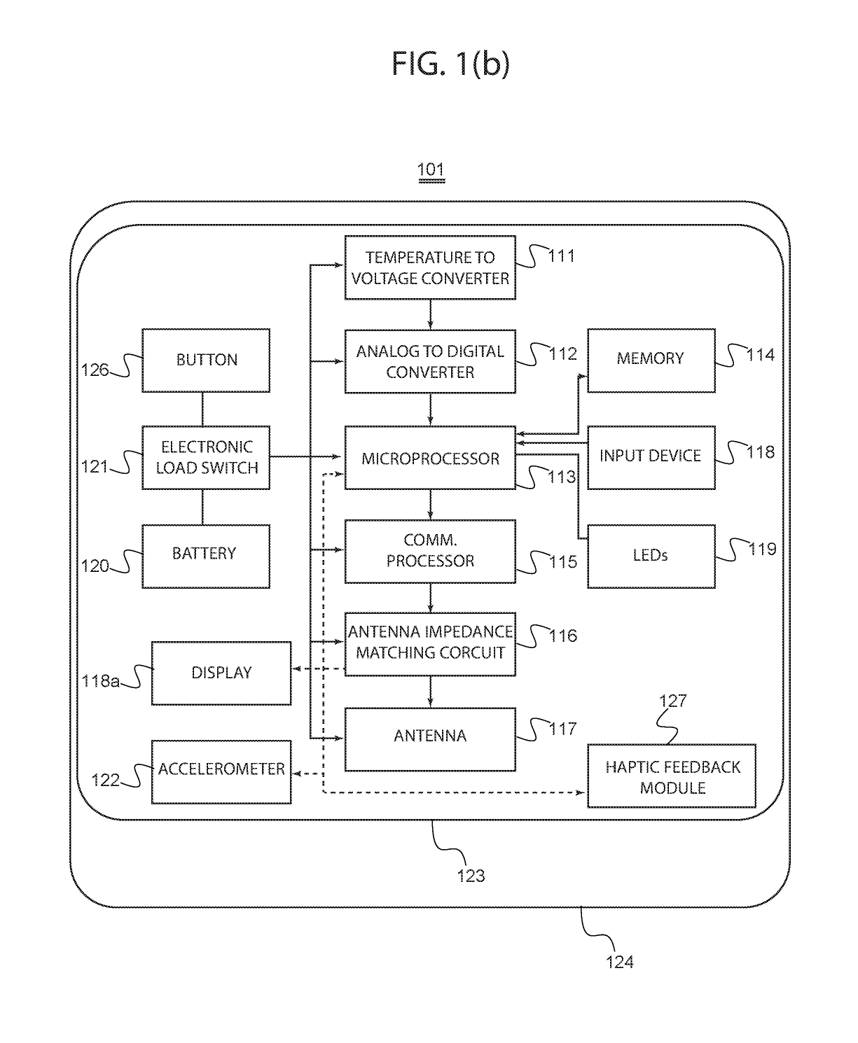

Turning now to the figures, FIG. 1(a) illustrates a system in accordance with an exemplary embodiment of the present invention. More specifically, FIG. 1(a) shows system 100, which comprises: wearable device 101 including a processing unit for executing programmable instructions 102 concerning sensor readings of one or more sensors of wearable device 101; and client devices 103 and 104, wherein client device 103 may include software or application 103a suitable to read and display to a user sensing data transmitted by wearable device 101. Moreover, in some exemplary embodiments, system 100 may further comprise: network 105 (e.g. Internet); and server 106 for storing user data in database 107 concerning the sensing data of a user of wearable device 101, which may be transmitted to client device 103 and communicated to server 106 via network 105. In some exemplary embodiments, server 106 may host a user interface 108 such as a web interface or website accessible to client devices, including for example client device 104, which may not necessarily receive data directly from wearable device 101, but may be used to access user's data provided by wearable device 101 from a remote location via access of website 108. Each of these components of system 100 will be discussed in turn.

Wearable device 101 may be have different structures and configurations including one or more sensors for gathering sensing data of a user. For example, and without limiting the scope of the present invention, wearable device 101 may comprise a wearable band, bandage, tape patch, or any other wearable structure including a garment that allows a user to attach, adhere or otherwise keep in close contact between the sensors of wearable device 101 and the user's skin. Generally, wearable device 101 includes circuitry configured to receive sensing data such as continuous temperature readings of the user or wearer of wearable device 101. A more detailed discussion of wearable device 101 follows below with reference to FIG. 1(b). Wearable device 101 may be configured for single continuous use (i.e. a 24-hour period) or for prolonged continuous use (several months) without deviating from the scope of the present invention, depending on the battery capacity and certain configurations. In exemplary embodiments, wearable device 101 is configured to be worn for prolonged periods of time and may be rechargeable.

Wearable device 101 is generally configured to continuously receive sensing data, for example body core temperature data, which may be stored locally. In exemplary embodiments, as will be discussed below, other data pertaining to an aspect of a user's health may be received via different sensors that may be included within wearable device 101 or in communication with wearable device 101, without limiting the scope of the present invention. In some exemplary embodiments, the continuously stored data may be shared by wearable device 101 with one or more client devices such as client device 103 via a short-ranged communication means such as local network 105a; in such embodiments, wearable device 101 may implement a communications processor configured for any type of local wireless network communications and may use any number of known technologies such as Wi-Fi™, Bluetooth™, ZigBee™, near field communication (NFC), or any other known protocol to establish a wireless personal area network (WPAN) or any suitable local network with a device such as client device 103. In other exemplary embodiments, the continuously stored data may be shared by wearable device 101 directly with a server, such as server 106, in order to store and back-up the data remotely and or to make the data available to the user or authorized users from a remote location via a device such as client device 104; in such embodiments, wearable device 101 may be configured to communicate via a communication means such as wide area network 105 using different technologies, for example cellular or using a locally available Wi-Fi™ network. In a preferred embodiment, wearable device 101 communicates with other devices only via a short-ranged communication means for security (and battery-conserving) reasons, and does not access a wide area network such as the Internet; any data that may be desirably stored in server 106 may be transmitted exclusively through an authorized device such as client device 103.

Programmable instructions 102 typically reside in a memory of wearable device 101. In exemplary embodiments, programmable instructions 102 may be configured to: generate sensing data (for example, and without limiting the scope of the present invention, generate temperature data); determine a state of wearable device 101; transmit sensing data to client device 103 or server 106; implement one or more algorithms for power management such as throttling communications, or other battery life preserving techniques; implement security protocols such as encryption protocols for transmitting user data securely or access protocols for allowing devices such as client device 103 to share a security key with other trusted client devices of the user; and any other instructions that may be suitable for proper functioning and or management of wearable device 101. A more detailed discussion of various algorithms that may be implemented via programmable instructions of a wearable device in accordance with the present invention, are presented below with reference to other figures.

Client device 103 may be any type of computing device such as a mobile device—including a smartphone or tablet—a laptop computer, a desktop computer, or even a proprietary device dedicated for communicating exclusively with wearable device 101. In exemplary embodiments however, client device is a smartphone suitable to communicate wirelessly with wearable device 101 via one or more communication channels as well as with server 106 via cellular or wi-fi communication means. For example, and without limiting the scope of the present invention, client device 103 may utilize short ranged communication protocols such as Bluetooth® in order to receive sensing data from wearable device 101, and access server 106 via a cellular network or wireless LAN to access the internet and transmit the user data to a depository situated at server 106 such as database 107. Typically, such smartphone will include a dedicated application, such as application 103a that facilitates visual and or auditable access to the sensing data and any other type of data including insights that may be provided to the user of wearable device 101 or otherwise an authorized user with permission to receive the user's data.

Application 103a may be any type of software application configured to receive data provided by wearable device 101 and provide the data to a user of client device 103, as mentioned above. In exemplary embodiments, application 103a may be a mobile application that may be downloaded on to a smartphone (client device 103) in a conventional manner. Typically, application 103a reads data from wearable device 101 and displays user parameters derived from this data to the user of client device 103—for example the wearer of wearable device 101 or an authorized user of the app. Providing the data may mean displaying the data via a display of client device 103 or providing an audio output via one or more speakers of client device 103. In exemplary embodiments, application 103a may synchronize user data with server 106, such as a server that may be located on the cloud. Generally, application 103a displays real-time information concerning the user data obtained from wearable device 101, although as will be discussed below several algorithms may be implemented to provide the user with useful determinations, insights or conclusions pertaining to a health state of the user based on the user data. Although in some embodiments, as will be discussed below, some of these tasks or determinations may be calculated by a processing unit of the wearable device, typically calculations and determinations based on the user data may be performed by external devices such as client device 103 via application 103a. For example, and without limiting the scope of the present invention, wearable device 101 may provide continuous temperature readings through a period of time of a user off the device, and application 103a may manage data concerning the temperature readings in order to store the data internally on client device 103 (or remotely on server 106) and may display the data in a useful manner such as by generating a graph of the stored data when sought by the user.

Application 103a may implement a myriad of features and functionality. In exemplary embodiments, application 103a may facilitate synchronization, notifications, data management, permissions, etc. For example, and without limiting the scope of the present invention, application 103a may provide up to date record keeping of data by seeking information it has not previously received from the wearable device; application 103a may also enable a user to select by what means the user wants to be notified; application 103a may enable a user to enter reminders that notify the user when the timing is appropriate for a specific action—such as reminders for a user to put on the device at a particular time in the day so that she does so before going to bed—examples of notifications may be text notifications or reminders. As an extension, it may allow the user to be notified on a separate device connected to the phone such as a smartwatch or an Amazon Echo. Other types of notification measures may be provided such as setting up alarms that detect whether wearable device 101 has been shut down, or whether application 103a may have been disabled; this may occur if the operating system resets or the application terminates. Naturally, a person of ordinary skill in the art will appreciate that other various means of notifications and reminders useful to users may be implemented without deviating from the scope of the present invention.

Network 105 is typically a wide area network, as mentioned above, for example the World Wide Web or Internet. Network 105 may be a means to remotely access server 106 in order for a user to access or otherwise manage their user data or provide access to another authorized user such as a physician or caregiver that may want to access the user's health data from a remote location. As such, network 105 is typically used for remote access, data management, or data backup as desired. As mentioned above, in some exemplary embodiments, no remote server 106 is required and remote data access is not part of system 100. In such embodiments, a user may simply access and manage their user data via client device 103, which typically communicates with wearable device 101 via a short-range communication network such as network 105a.

Network 105a may be any type of local wireless network and may use any number of known technologies such as Wi-Fi™, Bluetooth™, ZigBee™, near field communication (NFC), or any other known protocol to establish a wireless personal area network (WPAN) or any suitable local network which may be joined by a mobile device such as a smartphone, a tablet, lap top computer or any other mobile device such as client device 103 that has been configured and authorized to communicate with wearable device 101.

Server 106 may be configured with any known techniques and in any known manner to achieve a desired security and functionality. In exemplary embodiments, server 106 is a World Wide Web (WWW) server connected to the internet accessible via network 105. For example, and without limiting the scope of the present invention, server 106 typically includes one or more computers suitable for connecting to network 105. Optionally, server 106 may provide authorized entities, for example a user's physician, access to user data by means of a standardized interface, such as an application programming interface (API), web service, a website, or any other user interface 108, which may facilitate management or access to user data via client device 104 of system 100. In an exemplary embodiment, server 106 may comprise a two-tier server setup with one database layer and one application (web server) layer. This may allow the system to scale user wise by load balancing the application layer and also provide increased security. Without deviating from the scope of the present invention, a cloud based system may run on servers in the Amazon Virtual Private Cloud environment. Database backups may be encrypted and stored in Amazon's Simple Storage Service (S3). All security and firewalls may be configured using best practices and industry standard methods. Access to servers may be limited and monitored. All system access attempts (success or failure) may be logged in the database and physical log files as well as access to any base entities.

Database 107 may be similarly implement with known database architectures to store user data provided via wearable device 101 as well as any insights, determinations, calculations or other data that may be derived via application 103a of client device 103 such as charts, tables, notifications, settings and other useful information pertaining to a user of wearable device 101. Database 107 may hold multiple databases containing data objects within data repositories collected by server 106. The databases may be created by a known database manager using known technologies. In one embodiment, server 106 relies on cloud computing and database 107 may include technologies similar to those offered by Amazon™ such as Amazon™ Elastic Compute Cloud (AWS EC2), whereby database 107 may employ MySQL™ and AWS EC2™ instances.

As mentioned above, system 100 may optionally implement user interface 108, which may be a website, a web service or any type of application programming interface that provides users with access to the data collected by wearable device 101 and any insights generated through application 103a and provided to server 106 via client device 103 or that may be optionally generated by user interface 108 and stored in database 107.

Overall, system 100 centers around the electro-mechanical design of wearable device 101 to facilitate antenna function and transmission of heat from the user's body to one or more sensors of wearable device 101. As will be discussed in turn, wearable device 101 comprises of several components. FIG. 1(b) illustrates a diagram of several of these components as well as a flow chart of a method of providing temperature sensing data in accordance with practice of an exemplary embodiment.

Referring to FIG. 1(b), wearable device 101 may comprises of: temperature sensitive device or temperature to voltage converter 111, which measures temperature of a user; analog to digital converter 112, which converts the measurement to a digital signal; microprocessor 113 for processing the digital signal into a data payload; memory module 114, which in exemplary embodiments typically (although not necessarily) comprises non-volatile RAM memory for storing sensing data and or executable instructions such as programmable instructions 102; and communications processor 115 for transmitting temperature data through antenna impedance matching circuit 116 and antenna 117. Wearable device 101 may be powered by battery 120, through an optional electronic load switch 121, which gates the battery power from the battery (under a microprocessor or user controlled switch). In exemplary embodiments, wearable device may include an input device 118 that may provide a variety of functions, including for example a means to reset or turn off wearable device 101. In one embodiment, a single switch (or button 126) controls system power as well as input to the Microprocessor (as opposed to two separate switches). In some exemplary embodiments, wearable device 101 may further include additional user interface elements such as light emitting devices or LEDs 119 to provide indications to the user.

A temperature sensitive device such as temperature to voltage converter 111 may include one or more temperature sensors of an adequate size and sensitivity suitable for implementation into a circuitry of wearable device 111. Whatever configuration of the one or more sensors, temperature to voltage converter 111 is preferably situated on a layer of a flexible printed circuit board (PCB 123) with access to a circuit contact region that contacts the user's skin; as will be discussed in more detail below with reference to other figures, in exemplary embodiments, temperature to voltage converter 111 is preferably configurable to connect with the users skin via several printed pathways such as vertical interconnect access or vias between the layers of PCB 123. In one embodiment, two temperature sensors may be separated out such that one is in close thermal contact with the skin (sensor 1) and the other (sensor 2) is in better thermal contact with the ambient. The difference in temperature between sensor 1 and sensor 2 may be used to calculate the gradient of heat dissipation, which may be used to derive an accurate measure of skin temperature when the housing of the device is not thermally insulating. The combination of sensor 1 and sensor 2 may also be used for detecting faulty application of the device or when the device has fallen off the body.

Analog to digital converter 112 may be any device suitable for converting sensing signals to digital signals that may be read by microprocessor 113. Although shown as a separate component, a temperature sensing device that includes such converting means does not deviate from the scope of the present invention.

Microprocessor 113 may include one or more processing units suitable for executing programmable instructions 102. Accordingly, microprocessor 113 may be configured to: receiving temperature data from temperature sensitive device 111 on flexible PCB 123 that is folded to form multiple layers, wherein temperature sensitive device 111 is situated on a first layer of PCB 123, including a circuit with a contact region etched on a second layer of the PCB 123, the circuit including one or more pathways or vias connecting the contact region of the circuit to the temperature sensor; generating one or more data packets associated with the temperature data; and sending via an antenna situated on a second layer of the flexible circuit, the one or more data packets associated with the temperature data to a client device, such as client device 103.

As mentioned above, in exemplary embodiments, typically memory 114 comprises non-volatile memory or NVRAM suitable for storing the temperature data received from temperature sensing device 112 as well as suitable for storing programmable instructions 102. However, a person of ordinary skill in the art will appreciate that other memory configurations may be possible without deviating from the scope of the present invention (including combining the NVRAM with Processor)

Communication processor 115 and antenna impedance matching circuit 116 may be separate components or part of a single communications module without limitation. In an exemplary embodiment, a wireless interface may be provided and configured to implement Bluetooth or Bluetooth Low Energy or wireless LAN or Near-Field Communications (NFC for short) or cellular or LoRAN or a combination of two or more communications systems.

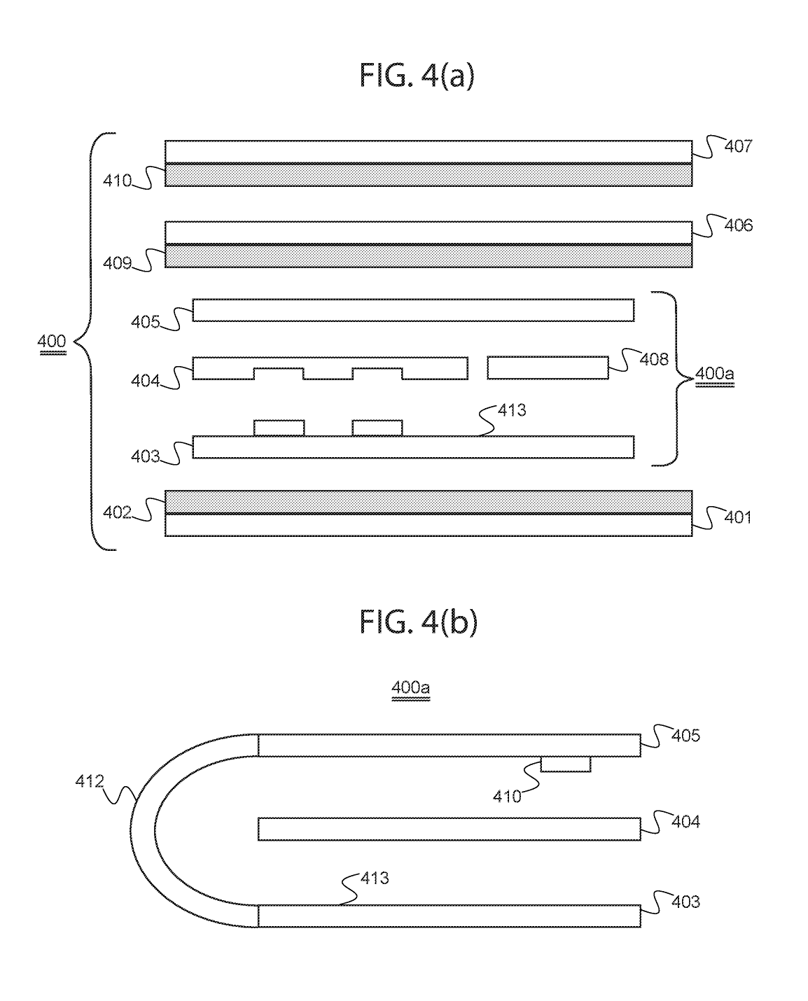

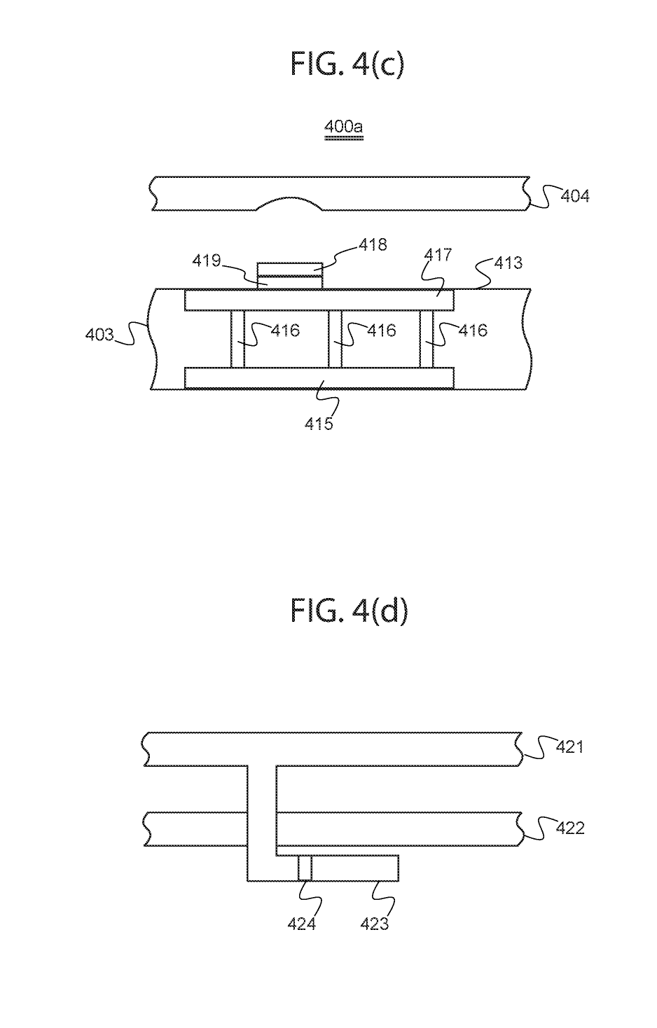

Antenna 117 may be any suitable antenna for transmitting user data captured by temperature sensing device 114 or any other sensing unit available to wearable device 101. In exemplary embodiments, antenna 117 is strategically placed on one of the layers of flexible PCB 123, which is preferably folded so as to allow placement of antenna 117 on a first plane of a layer of PCB 123, and placement of the ground (to antenna 117) on a different (opposite) plane of the PCB 123. A more detailed discussion of such exemplary antenna configuration is discussed with reference to FIG. 4(b).

Input device 118, as mentioned above, may be any type of input such as a simple button. This may be implement as a simple actuator device or in more complex embodiments input device 118 may include a touch interface part of a display (optionally shown as 118a) of wearable device 101.

LEDs 119 may provide a visual indicator to a user, especially in embodiments of wearable device 101 that do not include a display. Multiple color LEDs may provide useful feedback including but not limited to: a power (or battery usage) status of wearable device 101; a communication status (i.e. whether transmitting, not transmitting, accessible, airplane mode, etc.) of wearable device 101; a temperature status or threshold; or any other useful status that may be conveyed visually through simple LEDs to a user of wearable device 101; these indication can be distinguished by using multiple colors and blinking. In exemplary embodiments, LEDs 119 may be a means to alert the user by blinking—for example and without limiting the present invention—red, on a surface enclosure or housing of wearable device 101. This may be particularly useful if wearable device 101 has detected that there are no smartphones or tablets in communication range, and a need for alerting a status to the user is desirable. In some exemplary embodiments, as a careful tradeoff between power consumption and alerting, LEDs 119 may be kept blinking for a short duration before wearable device 101 is shut down. In some exemplary embodiments, when the wearable device detects that sensing or communications functions in the wearable device are not working or that, for example, client device 103 is not in range of wearable device 101, then LEDs 119 may indicate that a malfunction has occurred. Malfunction may also indicate that the battery is low, meaning the device should be recharged or discarded if the battery is not rechargeable. The indication may be executed for a short, fixed duration to ensure that the battery does not drain fully.

Moreover, in some exemplary embodiments, LEDs may be useful in providing self-test information indicative of a malfunction. Detecting a malfunction in the wearable device is necessary for safe and reliable use of the device. In the present invention, this may be achieved by means of a self-test that checks sensing, communications, and security functions in the wearable device. The process may begin during production of the device. During production, a test maybe implemented to run and store the identification information of major components in of wearable device 101. This may include the Electronic Serial Number of the sensor or the device ID in the processor. In active use of wearable device 101, a self-test may be run every time the processor is powered up and then, the test may be run periodically. This test checks to see if the processor is connected to components and if the components identify themselves as the ones in information that was stored during production. A break in the connection to the components or a mismatch in any of the parameters read, indicates that the device has lost hardware integrity and thus has malfunctioned. In some embodiments, an indication of malfunction may be broadcasted to a client device such as client device 103. If it is so detected that the device is unable to broadcast its condition, then the device may use LEDs 119 to indicate that the device is malfunctioning. In exemplary embodiments, after communicating the state of malfunction, the device may erase all user data on memory 114 and shut itself down. Moreover, in exemplary embodiments, the pattern emitted by the LEDs may be used to identify the specific malfunction. For example, a series of ON signals may identify that the malfunction code is equal to the number of times the LEDs turned on.

Moreover, although wearable device 101 is disclosed as including LEDs 119, in other exemplary embodiments, these may be replaced or used in conjunction with actuators that provide tactile and or auditory feedback to the user that is wearing the device. In exemplary embodiments, a haptic feedback module may be used whereby a vibrating component such as a vibration motor or a linear resonant actuator is driven by microprocessor 113 or by a dedicated hap tic driver chip (not shown).

Battery 120 may be a primary cell or a rechargeable battery or an energy harvesting circuit, without limiting the scope of the present invention. Furthermore, load switch 121 may be any type of load switch, relay or may be replaced by a power management integrated circuit. In an exemplary embodiment of wearable device 101, battery 120 is rechargeable; a wireless charging coil may be added to the circuitry of wearable device 101 so that battery 120 may be charged by an external charging mechanism without the use of exposed electrical contacts. In other exemplary embodiments, a separate charger may consist of a charging coil, a charging circuit and a USB voltage adapter that may be used to charge rechargeable battery 120 in wearable device 101.

Accelerometer 122 may be implemented in some exemplary embodiments of wearable device 101. In such embodiments, accelerometer 122 may be connected to microprocessor 113 as a means to measure the motion of the subject body to which wearable device 101 is connected. In such embodiments, wearable device 101 may provide motion data along with the temperature data, which may be useful for detection of an individual's health using their respiration rate and their body temperature.

Although not shown in this figure, other sensors and sensing devices may be incorporated with wearable device 101 without deviating from the scope of the present invention. For example, in addition temperature sensing device 112 and accelerometer 122 other sensing devices such as a heart rate sensor and device that records the user's pulmonary ventilation may be worn on the user's body and configured for communication with wearable device 101. In such embodiments body temperature detection may be combined with respiration rate and heart rate to detect a sudden motion of the user such as a fall and subsequent health condition.

As briefly mentioned above, PCB 123 is a flexible printed circuit board that is folded so as to create multiple layers. In exemplary embodiments, PCB 123 is folded with one of the folds creating a layer for housing antenna 117 and layer for housing the ground for antenna 117. This electrical layout of antenna 117 and its ground plane create a well-grounded radiating structure that can withstand the proximity to human tissue; this is very useful for a device that may be placed at the axillary region of the human body where temperature readings are medically accepted as accurate, and where incidentally the cavity and tissue of that region tends to attenuate radio frequency signals. In such embodiment, the configuration of the grounded layer or ground plane makes the resonant structure of antenna 117 less susceptible to detuning caused by the proximity of skin.

Similarly, one of the layers of PCB 123 may house temperature sensing device 112. Typically, this is a layer that is parallel to a layer that makes contact with or is closest to the skin of the user. In exemplary embodiments, the sensor may include a circuit or structure, such as a copper structure, which may utilize multiple copper vias in PCB 123 to conduct heat from the body (captured at the contact end situated on the layer closest to the skin) to the sensor situated on the parallel layer. In exemplary embodiments copper may be utilized because copper is highly conductive and the conductivity of copper is more than 100 to 200 times the conductivity of any plastic; essentially the copper area acts as a large receiving pad for the thermal heat flux from the skin. Of course, other materials may be used without deviating from the scope of the present invention.

PCB 123 may be situated on or incorporate a supportive layer such as a foam substrate housed within an enclosure 124. The supportive layer may help provide structural support as well as a means to make wearable device 101 more easily adjustable for placement against the body of a user. For example, and without limiting the scope of the present invention, wearable device 101 may incorporate a foam layer for supporting PCB 123 as well as a cloth enclosure that protects PCB 123 and makes wearing these components more comfortable to the user. Naturally, such enclosure 124 may include a means to be worn by the user, including but not limited to an adhesive that is suitable for application to the skin or any other structure such as bands, straps, etc. that may be useful in facilitating the device being worn. In exemplary embodiments, an adhesive is implemented on an enclosure 124 so that wearable device 101 may be placed and affixed at or near the axillary region of a user.

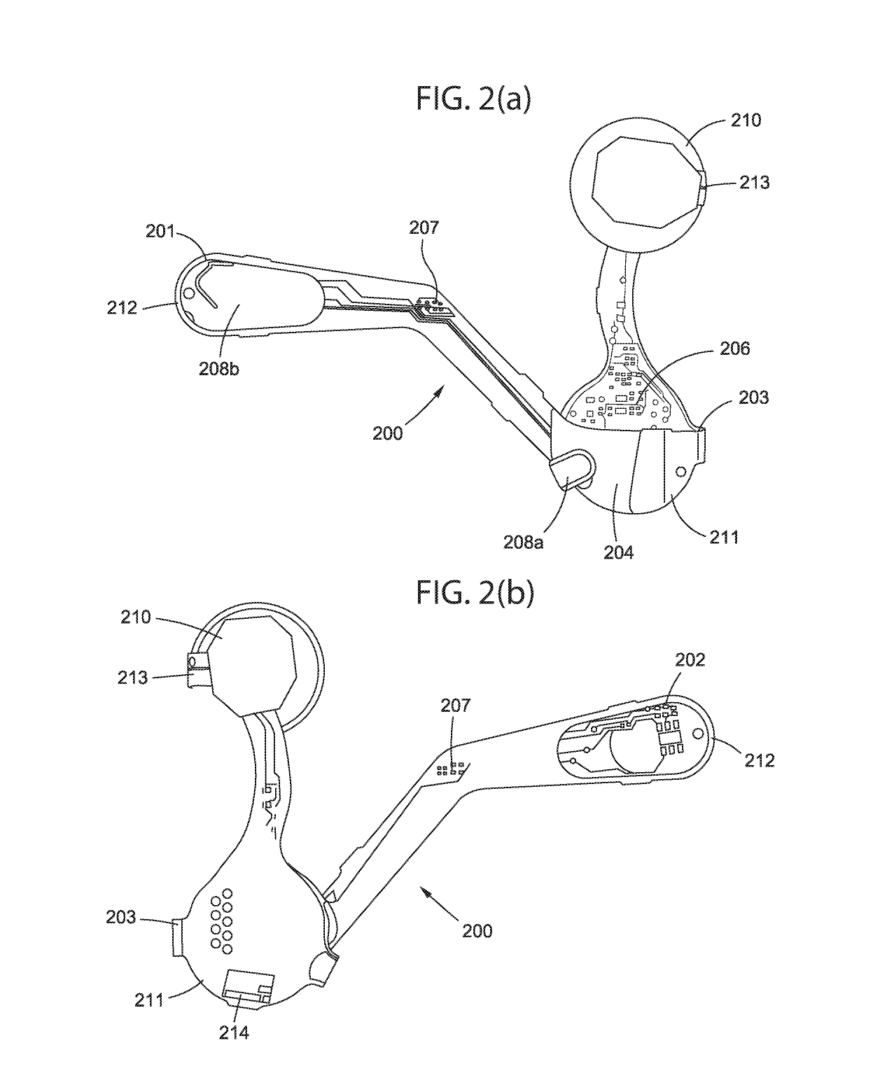

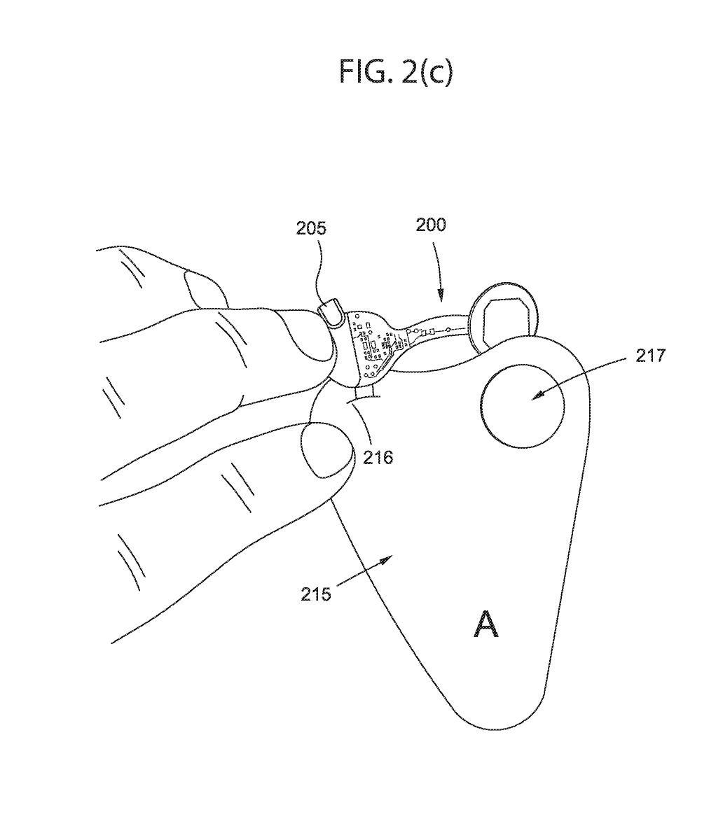

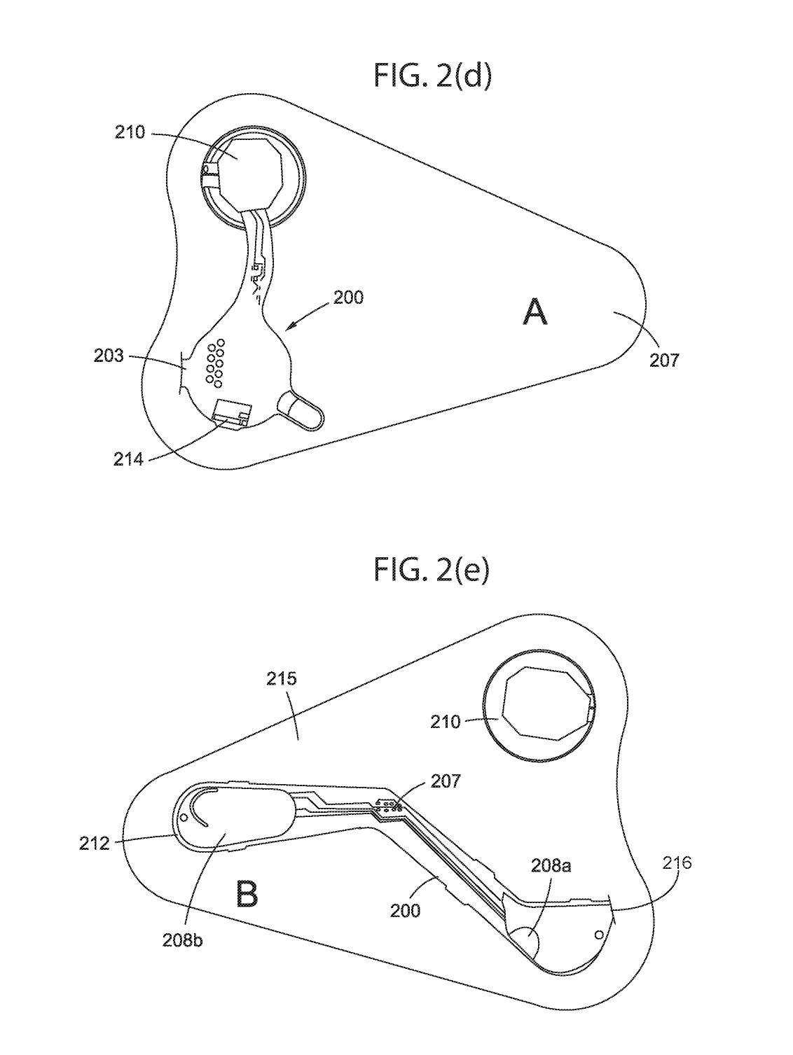

Turning now to the next set of figures, FIG. 2(a) depicts a bottom view of a flexible PCB for a wearable device in accordance with an exemplary embodiment of the present invention; FIG. 2(b) depicts a top view of the flexible PCB; FIG. 2(c) depicts the flexible PCB partially coupled to a foam layer in accordance with an exemplary embodiment of the present invention; FIG. 2(d) depicts a top view of the flexible PCB partially folded over the foam layer depicted in FIG. 2(a); and FIG. 2(e) depicts a bottom view of the flexible PCB partially folded over and threaded through a portion of the foam layer depicted in FIG. 2(a).

More specifically, these figures show PCB 200 comprising a first layer that expands across a plane having a first surface 201 (on a first side of the plane) and a second surface 202 (on the opposite side of the plane). Moreover, PCB 200 has been folded at fold 203 to form a second layer on a second plane that is substantially parallel to the first plane. The second plane has a first surface 204 (on a first side of the second plane) and a second surface 205 (on a second side of the second plane).

On surface 201 several components may be situated, including; circuitry 206 which may comprise of a microprocessor, memory, temperature sensor and communications module; LED circuitry 207; and contacts 208a and 208b. As may be appreciated from these figures, PCB 200 includes an elongated flexible substrate with at least three terminal ends 211, 212 and 213. A battery 210 may be coupled to PCB 200 at terminal end 213. An antenna 214 may be situated on surface 205 (FIG. 2(b)) of PCB 200.

PCB 200 may be coupled or even threaded through a plastic layer such as a 3M© 1772 foam layer 215 as shown in FIG. 2(c)-(e). In these views, it may be appreciated that terminal end 212 of PCB 200 has been inserted through an opening or slit 216 of foam layer 215, and unfolded about fold 203 to expose second surface 205 of the second plane formed by fold 203. Moreover, foam layer 215 includes an aperture 217 for registering with battery 210. Also from these views of FIG. 2(d) and in the view of FIG. 2(e), a surface A of foam layer 215 is visible. PCB 200 has been coupled or registered with foam layer 215, the elongated portion of surface 201 including terminal end 212 situated against surface B as shown in FIG. 2(e). PCB 200 is shown completely registered with foam layer 215, including contact 208a, which has also been threaded through the opening slit 216 of foam layer 215.

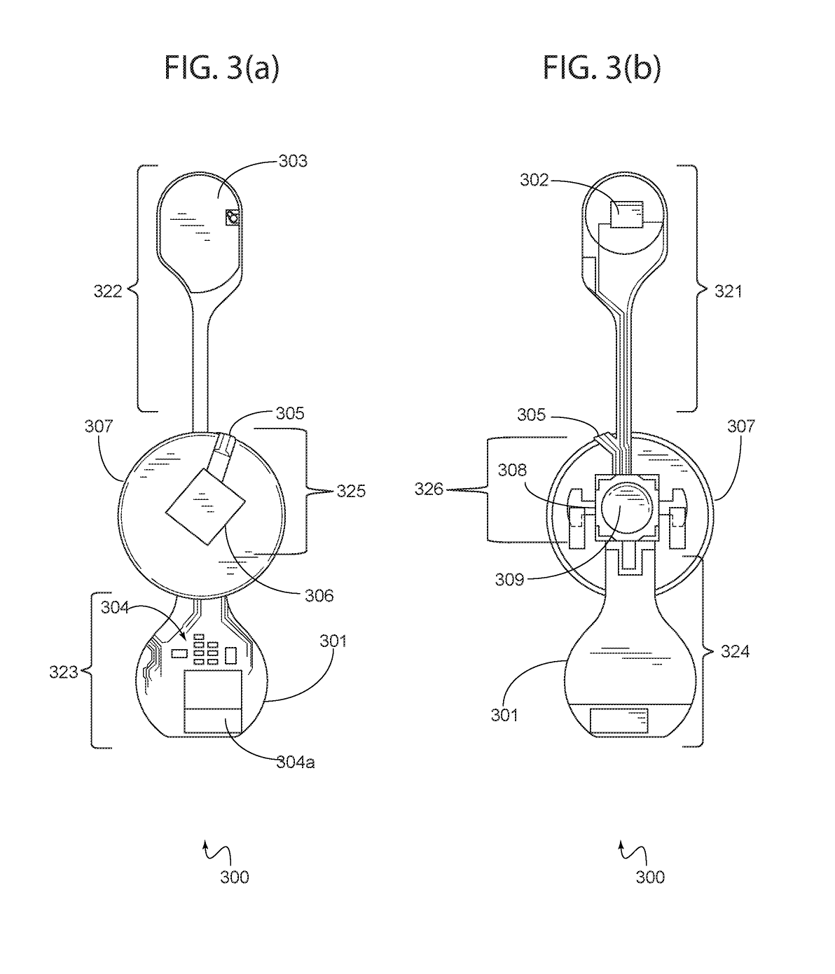

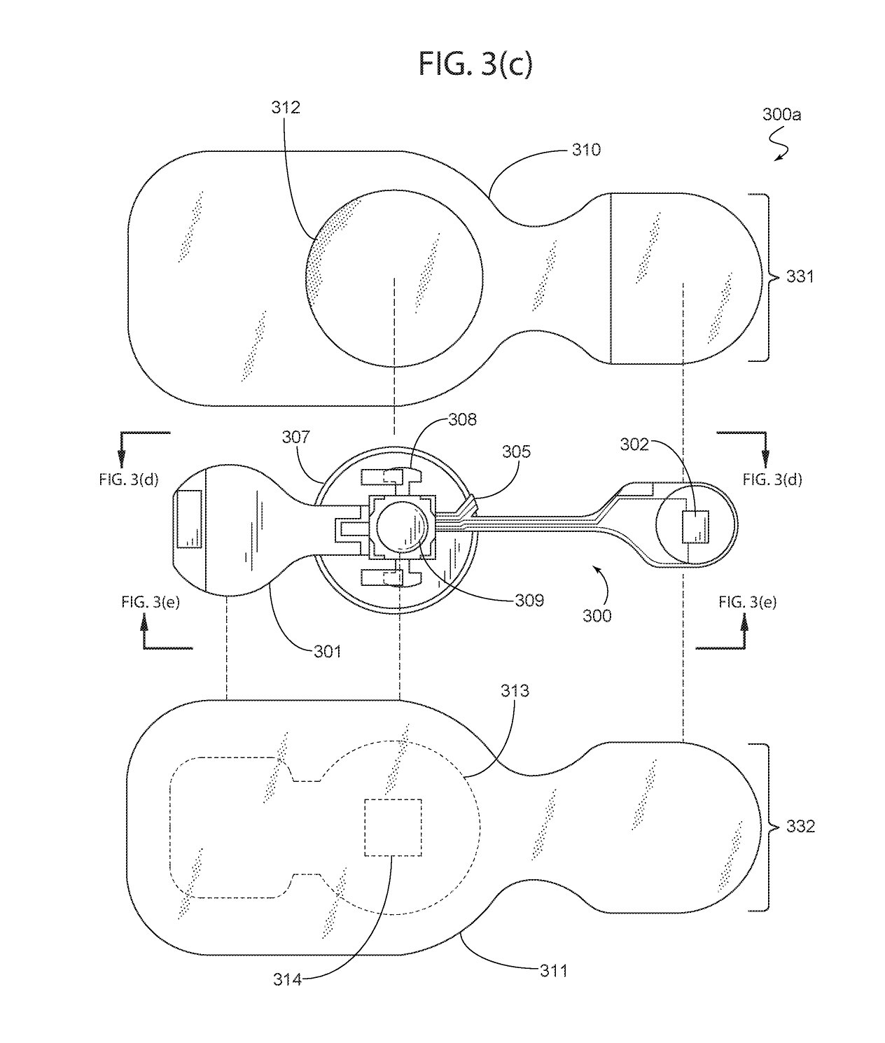

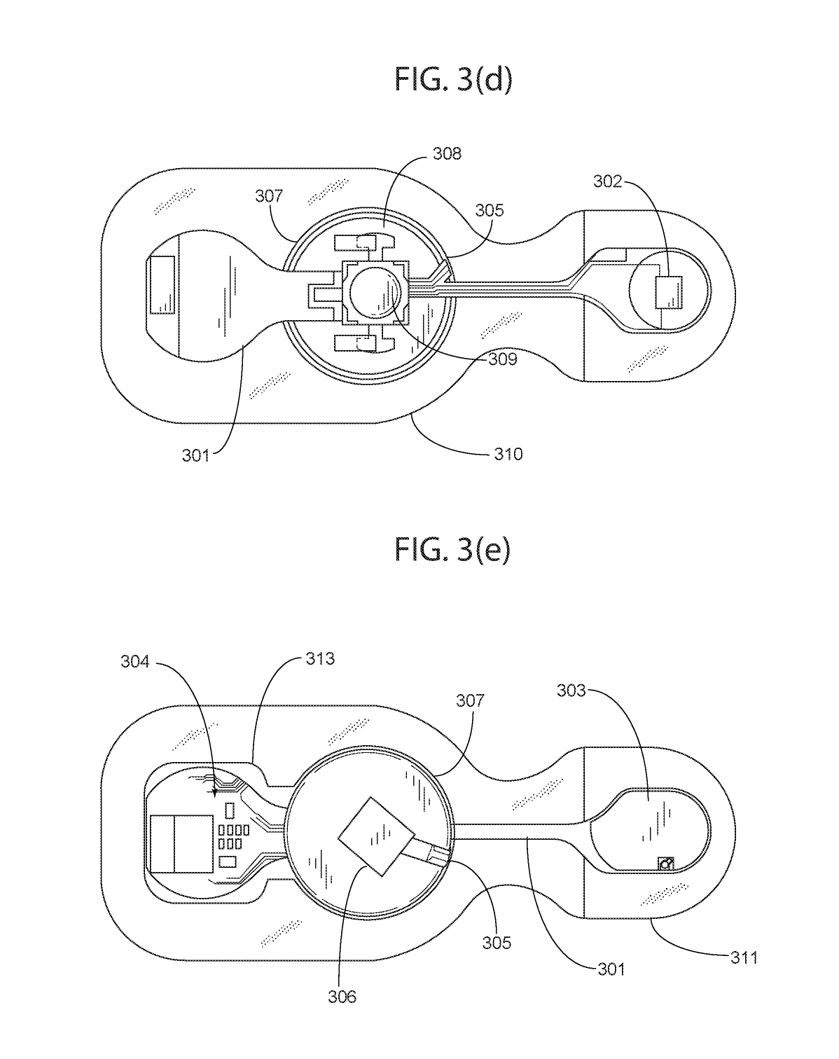

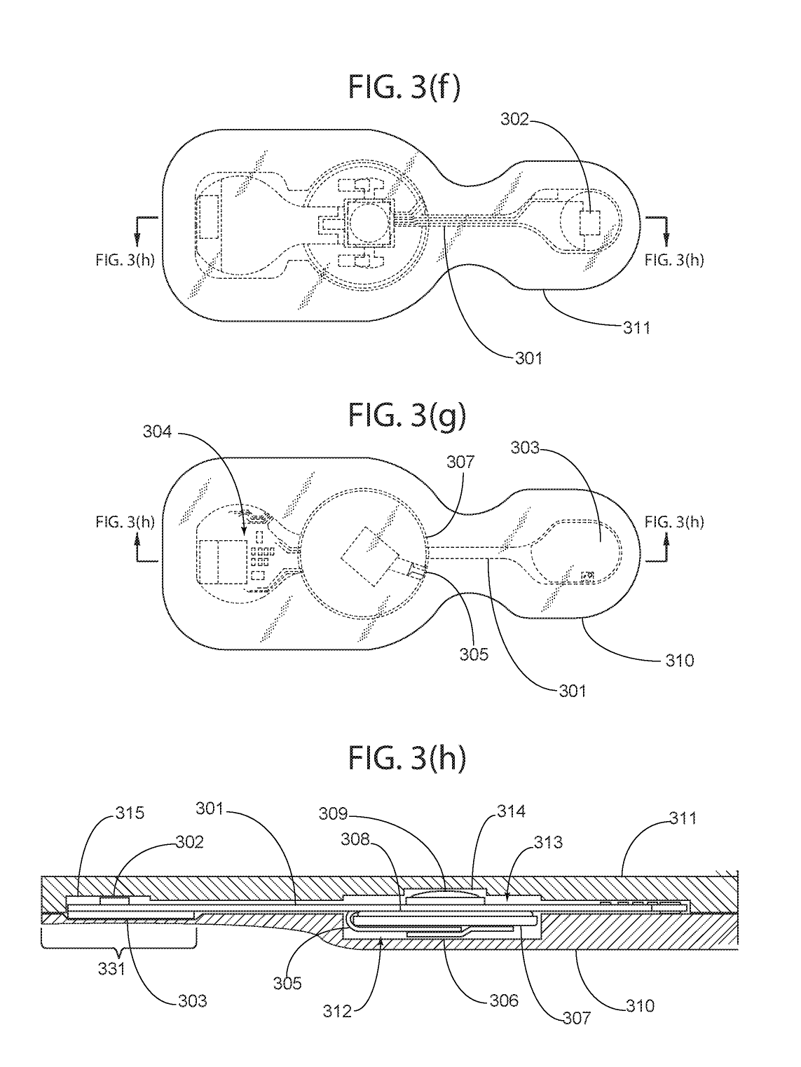

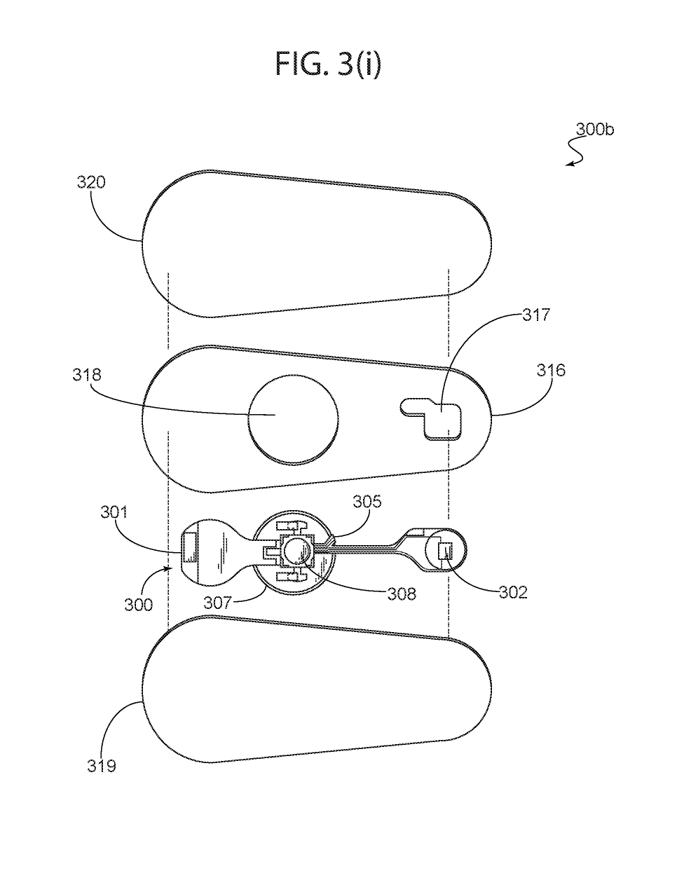

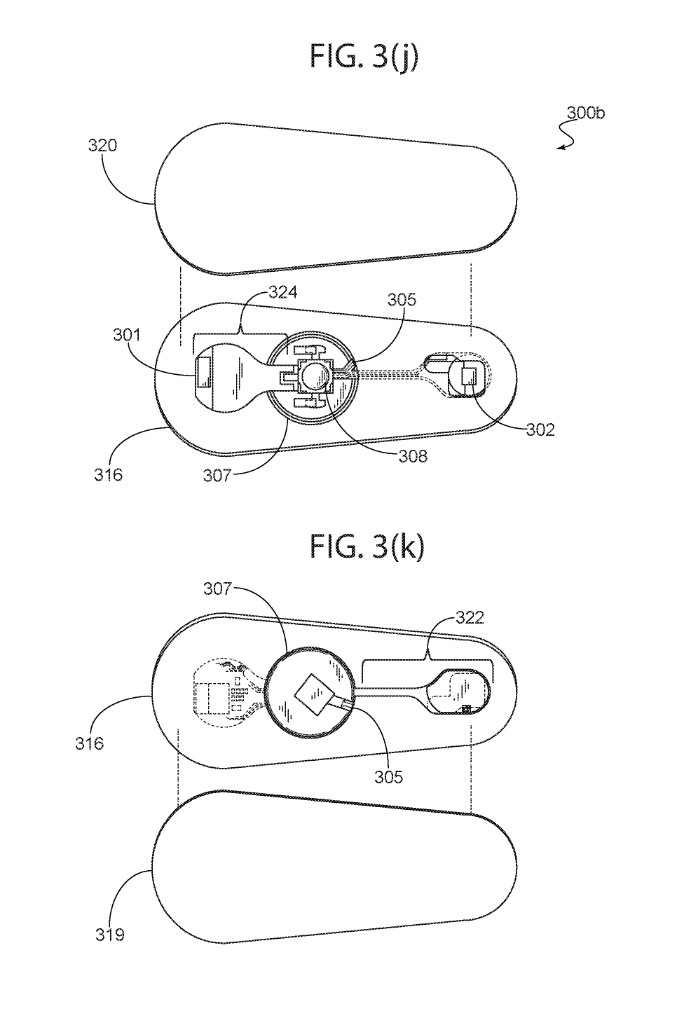

Now turning to the next set figures, another exemplary embodiment of a PCB for a wearable device in accordance with the present invention is discussed. FIG. 3(a) depicts a bottom view of a flexible PCB for a wearable device in accordance with an exemplary embodiment of the present invention; and FIG. 3(b) depicts a top view thereof. More specifically, these figures depict a printed circuit board (PCB 300) comprising a flexible substrate 301 that is folded to form multiple layers providing several surfaces 321, 322, 323, 324, 325, and 326); a temperature sensor 302 situated on a first surface 321 of a first layer of the PCB 300; a circuit including a thermal contact region 303 etched on a second surface 322 of the first layer of the PCB 300, the circuit further including one or more heat pathways (see for example FIG. 4(c)) connecting the thermal contact region 303 of the circuit to the temperature sensor 302; a communication module 304a including an antenna situated on one of the multiple layers (and in the shown embodiment on surface 323) of the PCB 300; and a microprocessor 304 (in the shown embodiment situated on surface 323) in communication with the temperature sensor 302 and the communication transmitter 304a, the microprocessor 304 configured to continuously obtain temperature sensing data from the temperature sensor 302 and transmit the sensing data to one or more client devices.

In exemplary embodiments, PCB 300 includes a fold 305 adapted to receive a battery for powering the PCB, the fold 305 connecting a circuitry of the PCB to a cathode 306 of the battery 307 and to an anode 308 of the battery 307. In exemplary embodiments, PCB 300 may further include a conducting adhesive to adhere a copper surface to terminals of battery 307, and a connection means from power rails to thermal contact region 303. In such embodiments, as shown in the current figures, fold 305 creates two opposite surfaces 325 and 326 that sandwich the battery 307 therein. Typically, a button 309 may be coupled along a surface 326 of the PCB 300 in order to, for example and without limiting the scope of the present invention, allow a user to turn on the device, activate or deactivate features of device, and or change operational modes.

In exemplary embodiments, an antenna ground may be situated on a layer different than the layer on which the antenna is situated such that the antenna and the antenna ground together act as a radiating structure for facilitating communication with the one or more client devices. In other exemplary embodiments, the antenna ground and antenna are situated on the same layer.

In some exemplary embodiments, the one or more heat pathways connecting the thermal contact region 303 of the circuit to the temperature sensor 302 comprise of a vertical interconnect structure (VIA) between the first layer and the second layer of the PCB 300, the VIA configured to conduct heat from the thermal contact region 303 to the temperature sensor 302. In exemplary embodiments, both the VIA and the thermal contact region 303 comprise of copper, such that a set of copper pathways (not shown here but see FIG. 4(c)) connect between sensor 302 on surface 321 with a copper layer etched on surface 322 that makes up the thermal region 303 on surface 322 of PCB 300.

In some exemplary embodiments, a thermally insulating adhesive may be applied on top of the temperature sensor 302 and the copper layer of the thermal region 303 to form a heat guide, the heat guide for drawing heat energy from a body of a user to the temperature sensor and minimizing a heat loss.

As will be better appreciated from other figures below, depending on the type of housing or enclosure employed by a wearable device in accordance with the present invention, PCB 300 may be disposed within a housing within a plurality of layers such as foam or plastic layers that are configured to secure and protect PCB 300 therein, but also to maximize a heat transfer between a user's skin and the temperature sensor 302.

For example, and without limiting the scope of the present invention, a first type of exemplary housing is discussed in turn with reference to FIG. 3(c)-FIG. 3(h). FIG. 3(c) depicts an exploded view of the flexible PCB 300, including a housing 300a in accordance with an exemplary embodiment of the present invention; FIG. 3(d) depicts a top view of the flexible PCB 300 placed inside a first portion of the housing 300a; FIG. 3(e) depicts a bottom view of the flexible PCB 300 inside a second portion of the housing 300a; and FIG. 3(f)-3(h) depict the wearable device fully enclosed in the housing 300a.

Turning first to FIG. 3(c), the exploded view shows housing 300a including a bottom enclosure 310 and a second portion such as a top enclosure 311, each portion configured to receive a portion of PCB 300 therein. In exemplary embodiments, enclosure 310 may include an aperture 312 suitable for receiving a portion of PCB 300, such as battery 307 coupled to PCB 300. Enclosures 310 and 311 may comprise any material suitable for securing PCB 300 therein, including but not limited to a foam material or a plastic material. For example, and without limiting the scope of the present invention, enclosures 310 and 311 may comprise a liquid silicone rubber, such as Dow Corning© QP1-250, having a silicone sealant disposed along an interior surface of each enclosure (i.e. for example around the edges). In exemplary embodiments, enclosure 311 includes an aperture 313, which further includes a smaller aperture 314 within aperture 313 for receiving a portion of button 309 of PCB 300 therein. In exemplary embodiments, a portion 331 of enclosure 310 may have a reduced thickness from a thickness of the entire enclosure 310 so that when PCB 300 lays on top or along a surface of portion 331 of enclosure 310, heat may be more easily transferred between the user's skin and the thermal region 303 that lays along said surface of portion 331 of enclosure 310. A portion 332 of enclosure 311 may have a similar gradual change in thickness or may be equal in thickness along an entire length of enclosure 311. In exemplary embodiments, an added thickness protects the thermal sensor 302 that lays along a surface of portion 332 of enclosure 311 so that the antenna of the device is on the same plane as sensor 302.

With reference to FIG. 3(d), in this exemplary embodiment of the wearable device, PCB 300 may typically lay within the bottom enclosure 310 such that a bottom portion of battery 307 (i.e. including cathode 306) is received within aperture 312, and a first surface 321 on a first layer of PCB 300 is exposed—thereby exposing thermal sensor 302. As such, a second surface 322 on the opposite side of the first layer of the PCB 300 will be situated against a surface of portion 331 of enclosure 310 of housing 300a. As may be appreciated from this view, fold 305—which sandwiches battery 307 within, creates a layer of the PCB 300 situated within aperture 312 with button 309 exposed on another layer of the PCB 300. Surface 324 of PCB 300 then lays substantially parallel with surface 321.

With reference to FIG. 3(e), in this exemplary embodiment of the wearable device, PCB 300 may typically lay within the top enclosure 311 such that a top portion of battery 307 (i.e. including anode 308) is received within aperture 313—and more specifically so that a portion of button 309 is situated within aperture 314 (within aperture 313) of enclosure 311. In this way, surface 322 on the first layer of PCB 300 is exposed—thereby exposing thermal region 303 (i.e. whenever enclosure 310 is decoupled from enclosure 311). As such, the first surface 321 on the opposite side of the first layer of the PCB 300 will be situated against a surface of portion 322 of enclosure 311 of housing 300a. As may be appreciated from this view, fold 305—which sandwiches battery 307 within, creates a layer of the PCB 300 situated within aperture 313 with button 309 also therein. Surface 323 of PCB 300 then lays substantially parallel with surface 322.

Now with reference to FIG. 3(f), FIG. 3(g), and FIG. 3(h), it may be appreciated from these views that when each enclosure 310 and 311 is secured to each other (for example using a silicone sealant to adhere the two enclosures), PCB 300 is sandwiched within housing 300a. Accordingly, in this exemplary embodiment, PCB 300 is secured within housing 300a so that surfaces 321 and 324 lay substantially parallel along the same plane on a top side of housing 300a with a portion of battery 307 and button 309 within cavity 313 and 314, respectively, and surfaces 322 and 323 lay substantially parallel on an opposite side of the same plane on a bottom side of housing 300a, with a portion of battery 307 and cathode 306 of battery 307 within cavity 312. From this view, it may be appreciated that thermal region 303 of PCB 300 may be disposed along a surface on portion 331 of enclosure 310 that comprises a thinner layer of plastic so as to facilitate the transfer of heat between a user's skin and sensor 302.

Now turning to the next set of figures, a second type of exemplary housing is discussed in turn with reference to FIG. 3(i)-FIG. 3(k). FIG. 3(i) depicts an exploded view of flexible PCB 300, which may be secured within a housing 300b in accordance with an exemplary embodiment of the present invention; FIG. 3(j) depicts a top exploded view of the flexible PCB threadedly secured inside a foam or plastic layer 316 of housing 300b with a top plastic or foam layer 320; and FIG. 3(k) depicts a bottom exploded view of the flexible PCB 300 secured to layer 316 with a bottom plastic or foam layer 319.

More specifically, in this embodiment, housing 300b differs from housing 300a in that housing 300b includes a middle support layer 316, onto which PCB 300 is threadedly secured thereto, which is sandwiched between a top layer 320 and a bottom layer 319. This construction may be flexible so as to conform to a user's body and facilitate a comfortable fit against the user when the wearable device is worn. As such, housing 300b may include a top layer 320 and a bottom layer 319, each which may comprise a foam material that is generally flexible and soft. Middle layer 316 may include a plurality of apertures 318 and 317 adapted to receive portions of PCB 300, so that PCB 300 may be secured within each aperture.

In an exemplary embodiment, and without limiting the scope of the present invention, bottom layer 319 and top layer 320 are planar layers with substantially smooth surfaces, each layer 319 and 320 having an elongated oval perimeter with substantially round edges. In some exemplary embodiments, such as shown in these figures, each layer 316, 319, and 320 has a terminal end that is slightly narrower than the opposite terminal end. This construction, including the softer materials, the rounded edges and the oblong, elongated perimeter with a narrower terminal end, facilitates a more comfortable wear by a user.

In the shown exemplary embodiment, middle layer 316 is a support layer that includes an aperture 318 in a middle portion along the length of layer 316, which is adapted to receive a portion of battery 307 of PCB 300. At one of the terminal ends, for example and without limitation. the narrower terminal end, a second aperture 317 may be adapted to receive sensor 302 within. This way, sensor 302 may be protected within the aperture 317 and also ensure that a user avoids feeling any non-flexible or less flexible components such as the battery and the sensor of PCB 300, when the wearable device is pressed against the skin.

With reference to FIG. 3(j) and FIG. 3(k), it may be appreciated that once threaded through these apertures 318 and 317, the surfaces of PCB 300 will be positioned slightly different than when housed in a housing such as housing 300a. For example and without limiting the scope of the present invention, where a first layer of the PCB 300 may include on a first surface 321 and an opposite surface 322 of the first layer of PCB 300, a second layer of PCB 300 may include a third surface 323 and an opposite surface 324 of the second layer of PCB 300, wherein PCB 300 is threaded through aperture 318 of layer 316 such that the first layer is situated on a different plane than the second layer. In the current figures, for example, it may be appreciated that surface 324 (of the second layer) is situated on an opposite side of layer 316 and thus on a different plane than surface 322.

Moreover, in exemplary embodiments, because thermal region 303 is placed against bottom layer 319, which in turn will be in contact with the user's skin in order to detect a temperature, layer 319 may comprise a thin layer so as to facilitate heat conductivity to sensor 303 via the heat pathways connecting thermal region 303 with sensor 302 of PCB 300.

Turning now to the next figure, FIG. 4(a) illustrates different layers of a PCB in accordance with an exemplary embodiment of the present invention, such as an embodiment in accordance with FIG. 2(a)-FIG. 2(e). More specifically, FIG. 4(a) depicts a wearable device 400 that comprises a flexible PCB 400a folded around and in between multiple layers of polymeric foam or plastic—this embodiment consistent with the embodiment disclosed in FIG. 2(a)-FIG. 2(e). In the shown exemplary embodiment, a flexible PCB 400a is folded at fold 412 (see FIG. 4(b)), which provides a connecting layer for an antenna's ground plane 413 between a lower layer 403 of the flexible PCB 400a, and an upper layer 405 of the flexible PCB 400a. A base layer 401, typically closest to the skin, may be made of foam or plastic that is constructed to be thermally conducting and electrically insulating. This layer is attached to the lower layer 403 of flexible PCB 400a by means of a thermally conducting adhesive layer 402. The lower layer 403 of flexible PCB 400a houses electronics, circuit traces and the connection to the battery 408. The lower layer 403 of flexible PCB 400a may be attached to a middle layer 404 that may be made of foam or plastic that constructed to provide thermal insulation and physical rigidity for wearable device 400. In the shown exemplary embodiment, the middle layer 404 is then attached to the top layer 405 of flexible PCB 400a. Moreover, middle layer 404, which is connected to the battery 408, contains or houses the antenna 410 within (for example as seen in FIG. 3(a)-(d)). The top layer 405 of flexible PCB 400a may be attached, using adhesive layer 409, to an additional layer 406 that may be similarly constructed of foam or plastic, which similarly provides thermal and electrical insulation as layers 401 and 404. Layer 406 may be attached to a label 407, which provides product labeling, using adhesive layer 410. The battery 408 may be situated between the two layers 403 and 405 of flexible PCB 400a, for example—as mentioned above—situated on or within an aperture foam layer 404. See for example aperture 217 of the embodiment disclosed with reference to FIG. 3(a). The folding of the PCB 400a about fold 412 surrounds the foam or plastic layer 404, which is an electrically non-conducting and thermally insulating layer between the layers of PCB 400a. In an exemplary embodiment, electrical connections to the battery may be made using conductive adhesive fabric that may be stuck to the flexible PCB copper pad or copper area and the battery. Alternatively, copper area may be welded to the battery.

Turning to the next figure, FIG. 4(b) illustrates a side view of folded PCB 400a and the different layers formed therein in accordance with an exemplary embodiment of the present invention. More specifically, FIG. 4(b) illustrates a sub-structure, in which antenna 410, is placed in the top PCB layer 405 formed when the PCB 400a is folded along fold 412, thereby situating a ground plane 413 at the bottom layer 403 of PCB 400a. As mentioned above with reference to FIG. 4(a), the two layers (403, 405) of PCB 400a are separated by a foam or plastic layer to maintain a consistent separation 404. The antenna 410 and the ground plane 413 together act as a radiating structure for communication with a device at the frequency of operation. In exemplary embodiments, the separation between the two layers, substantially equals to the thickness of the foam layer 404. For example, and without limiting the scope of the present invention, in exemplary embodiments, about 1.6 mm may be used in order to create a reliable radiating structure even when the device containing this sub-structure is attached to the body. A typical radiating antenna structure is built on a hard PCB that will typically have an antenna on one side and a ground plane on the other. This naturally gives a 1.6 mm (or a consistent amount of) separation between the antenna and ground because the hard PCB is 1.6 mm thick. Since the application requires the use of a flexible PCB, which is about 25-50 microns thick, it is not possible to create this separation using the flexible PCB as it is. Accordingly, the ground may be positioned between the resonant structure and the body.

As may be appreciated by a person of ordinary skill in the art, antennas receive energy from an RF transmitter and radiate it out. Antennas are tuned to the desired frequency of operation, but if the tuning of the antenna does not match the frequency of transmission (or reception), the antenna will reflect most of the energy that is fed to it, back to the source. This will result in a very small amount of energy to go out as radiation, and hence a very weak signal at the receiver. For wearable or on-body devices, the challenge with antenna design is the fact that the tuning of the antenna changes due to the proximity of the body; that is, there is a shift in the tuning when the antenna is placed against the body. This is due to the high dielectric constant of the human body which is largely water. (Water has dielectric constant of about 80, where as air has dielectric constant of about 1, most plastics have dielectric constant of 3 to 5). Accordingly, one function for the middle layer of foam is to provide separation between antenna and the ground plane to create a radiating structure that is robust to the distance from the body. The ground plane also acts as a shield to decouple the effect of the body from the resonant structure of the antenna by allowing the electric fields to terminate mainly on the copper ground instead of penetrating the body.

Turning now to the next figure, FIG. 4(c) illustrates a sensor and heat pathway of an exemplary PCB in accordance with the present invention. More specifically, FIG. 4(c) illustrates flexible PCB 400a folded to form multiple layers, wherein a temperature sensor 418 is situated on a first surface of layer 403 of PCB 400a, and a circuit, such as a copper circuit, includes a thermal contact region 415 etched on a second surface of layer 403 of PCB 400a, the thermal contact region 415 including one or more pathways 416 connecting the contact region 415 of the copper circuit to the temperature sensor 418, which effectively creates a heat pathway. This configuration helps reduce the time taken to accurately measure the core temperature by guiding body heat to the sensor using the conductive material (for example copper) in the PCB.

As shown in FIG. 4(c), which is a magnification of only the “heat guide” or pathway, the lower layer of the PCB contains a contact region 415, which in the shown exemplary embodiment may be made of copper that is connected by one or more pathways such as copper vias 416 that thermally connect opposite surfaces of layer 403 of flexible PCB 400a; one surface including copper plate 415, and another surface including copper layer 417. Copper layer 417 may be in physical contact with temperature sensor 418. Optionally, a thermally conducting adhesive 419 may aid in the attachment of copper layer 417 to temperature sensor 418. Further, optionally, a thermally insulating foam layer, such as layer 404, may be placed on top of the temperature sensor 418 to minimize heat leakage and hence the effect of ambient temperature.

In exemplary embodiments, a thermally insulating adhesive may be placed on top of the temperature sensor 418 and copper layer 417; the combination of thermally conducting bottom foam layer, thermally conducting adhesive, ground plane made of copper and copper vias and optional insulation top of the temperature sensor altogether comprise a heat guide that draws heat energy from the body of the user to temperature sensor 418 and prevents the heat loss to the ambient. With this configuration, temperature sensor 418 rapidly achieves thermal equilibrium with the body, thereby allowing for rapid measurement of the body's temperature. This is because, in order to reach thermal equilibrium, the temperature difference between the sensor, all elements in thermal proximity to the sensor and their heat capacity come into play. The novelty here is in the use of the two copper pads, one in the proximity of the skin and the other connected to the temperature sensing element and large number of copper vias between the two copper pads. The copper pad in the proximity of skin acts as a receiver of the heat flux from the body. The copper pad also conducts heat to the vias that connect the two copper pads. Even though the vias have a small cross-section, there may be many of them, allowing sufficient amount of heat to be transmitted to temperature sensor 418 in order to achieve temperature equilibrium in seconds rather than minutes. As mentioned briefly above, the heat pathway and heat guide discussed with reference to FIG. 4(c) may be employed with a wearable device utilizing a PCB such as PCB 300 rather than PCB 400, without deviating from or limiting the scope of the present invention.

In exemplary embodiments of the present invention, a second temperature sensor or even multiple temperature sensors may be utilized. An ideal placement for a second and subsequent temperature sensors may be on 405—the temperature gradient between the skin and temperature sensitive devices—in order to measure, during transients and in steady state, the temperature of the ambient; the first sensor measures the temperature of the skin. This would allow for a method to measure accurately the skin temperature even in the presence of heat leakage in the device. Another application of the second temperature sensor would be to calibrate the measurements made by the first temperature sensor. A further application of the combination of the two or more sensors is in the use of error or fault detection, error estimation and error correction while the device is being used for its primary function. A typical use of the combination of the two or more sensors is in self-test.

Finally, FIG. 4(d) illustrates a close-up view of another exemplary embodiment of a flexible PCB and foam configuration in accordance with the present invention. More specifically, FIG. 4(d) shows components of a flexible PCB 421, which may be folded so that they protrude out through notches into a bottom foam layer 422 and attach to metal contacts 423 over a copper area 424.

As mentioned above, a system in accordance with the present invention may include a wearable device that is configured to detect a voltage difference as well as or in the alternative to temperature data. A common example of a system of measuring potential differences within the human body is provided by the electrocardiogram (ECG), which refers to a plot against time of the varying potential differences existing between various standard electrode pairs positioned on the surface of the body. A conventional ECG measurement will include twelve signal measurements, also referred to as “leads”, that are taken using a set of standard electrodes pairs. Utilizing this same principle, it is possible that a wearable device in accordance with the present invention is configured to receive signals indicative of a plurality of voltage measurements of the human body, and generate voltage differential signals associated with the differences in said voltages, in order to generate voltage differential data that may be used, for example, by a physician such as a cardiologist to diagnose a particular condition.

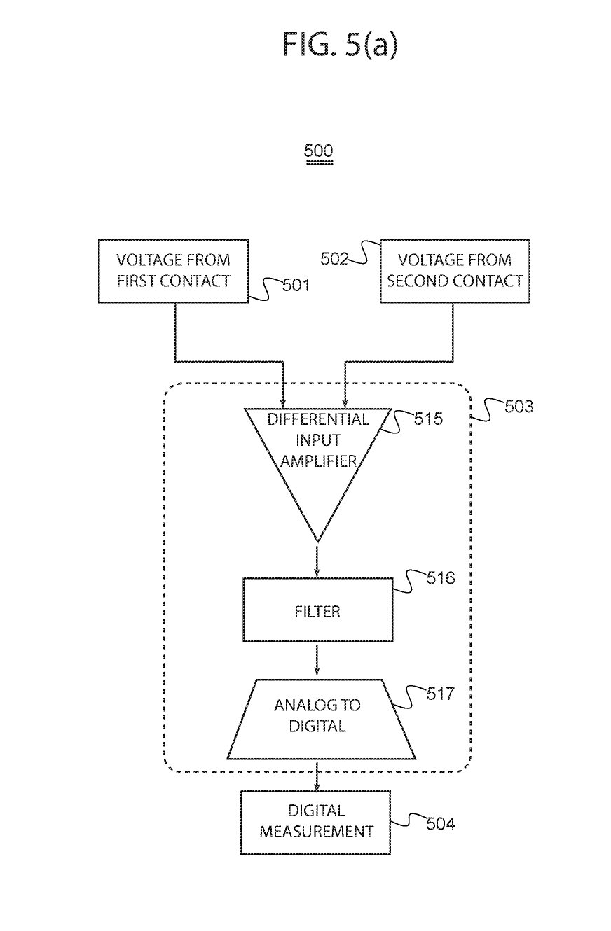

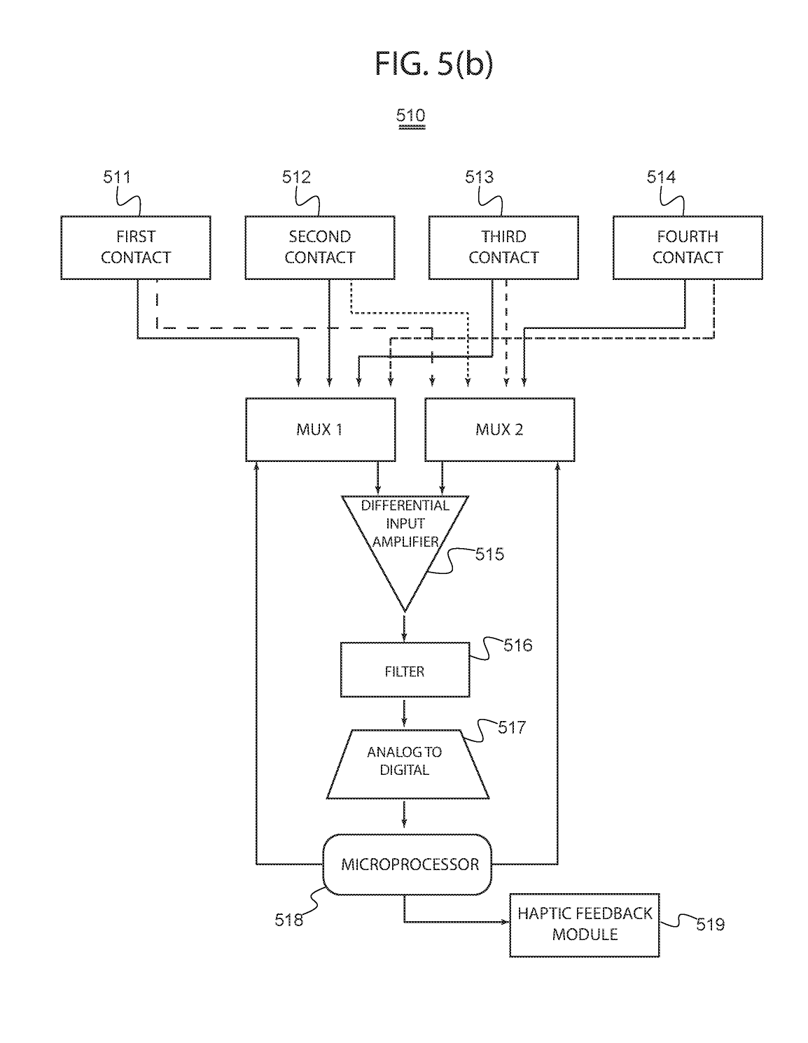

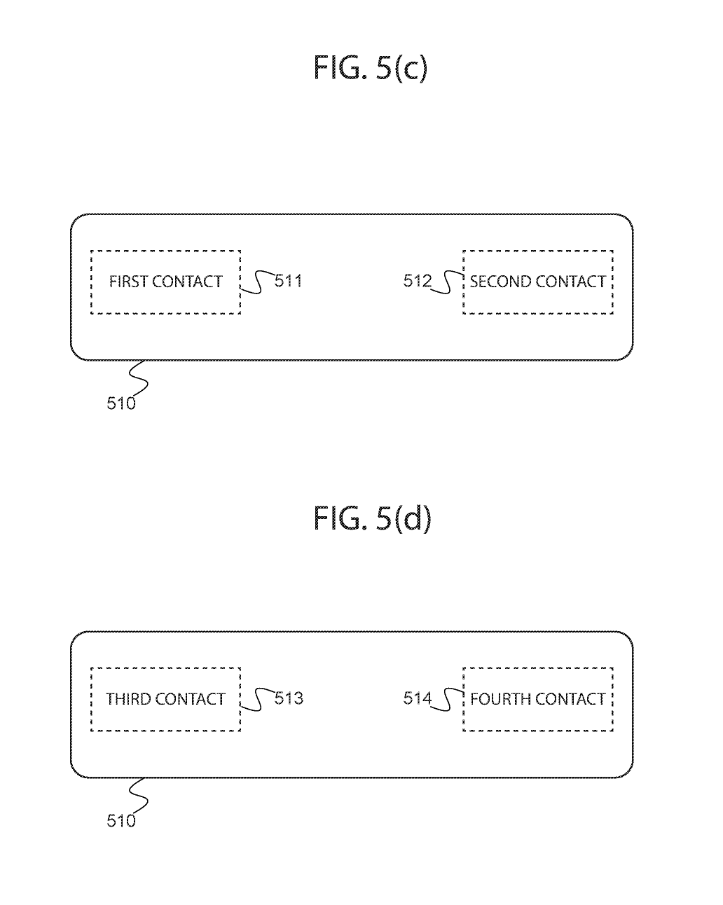

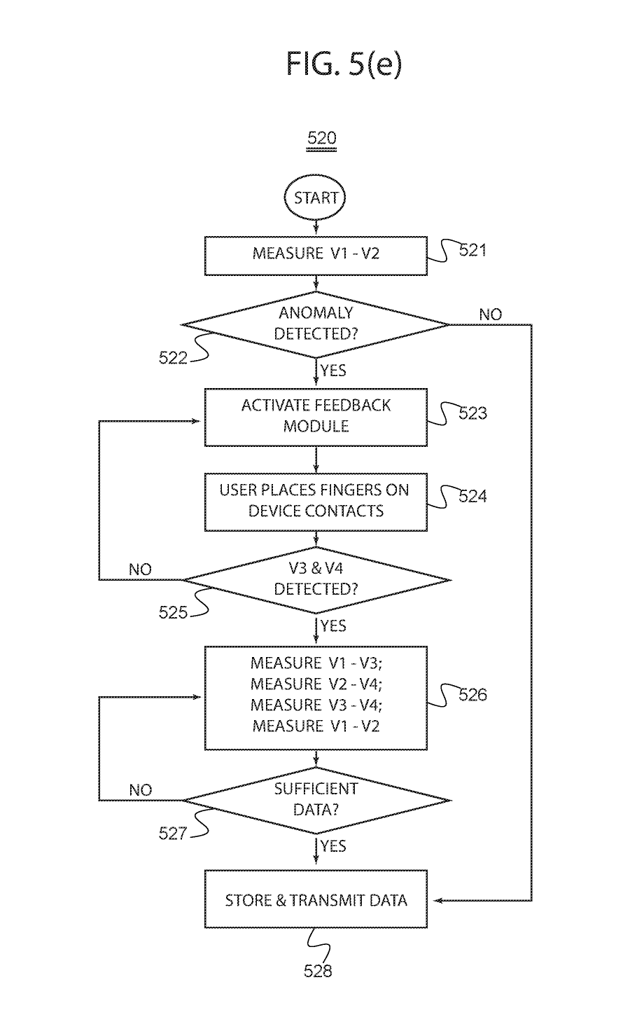

Next, FIG. 5(a) illustrates a flow chart of an exemplary method of reading potential difference data. More specifically, FIG. 5(a) shows method 500 for utilizing a voltage difference measured between two or more electrical contacts in order to generate a digital measurement. It is noted that although method 500 is shown in a particular sequence of steps, other conceivable sequence of the steps may be practiced without deviating from the scope of the present invention.

Generally, at step 501, voltage measured at a first contact, such as a first copper contact may be received. Similarly, at step 502, voltage measured at a second contact, such as a second copper contact may be received. In step 503, a voltage difference measured between the first contact and the second contact may be amplified by using an amplifier circuit including but not limited to a differential input amplifier 516, filtered using a filter circuit 516, and then converted to a digital measurement using an analog-to-digital-converter 517, at step 504 thereby producing a digital measurement that is derived from the input voltage difference. In another embodiment, the voltages from the metal contacts may be fed directly to the analog-to-digital convertor wherein the amplification and filtering may be done digitally.

In exemplary embodiments, the amplifier circuit may be combined with the filter circuit such as by using an operational amplifier with a filter in the feedback circuit. Further, in other exemplary embodiments, one or both of the contacts used for measuring the voltage difference may also be used as the heat collector for measuring temperature.

In some exemplary embodiments, a temperature measurement may be combined with the digital measurement of voltage difference and a combination of the two measurements may be created. In such embodiments, the PCB may be extended to connect to two electrical contacts that are either in direct contact with the skin or in contact to the skin through heat-conducting adhesive.

An exemplary application of measuring the voltage difference as in method 500 may be in the measurement of the heart rate, which may be computed from the voltage difference as the measure of periodicity of the electrical waveform. The combination of temperature and voltage difference thus leads into applications that use both the body's temperature and the heart rate such as in the measurement of the body's calorific output, the detection of physiological stress, the detection of psychological stress and the detection of shock. As such, a wearable device in accordance with the present invention may implement a method for measuring temperature and heart rate concurrently using a single wearable device.

Another exemplary application of measuring the voltage difference as in method 500 may be in the measurement of heart rate variability, which is simply a measure of the change in periodicity of the electrical voltage difference when the device is placed in the armpit. Measures of heart rate variability are temperature dependent in the range of therapeutic hypothermia to normothermia. Core body temperature needs to be considered when evaluating heart rate variability metrics as potential physiologic biomarkers of illness severity in hypoxic-ischemic encephalopathy infants undergoing therapeutic hypothermia. As such, the combination of core body temperature and heart rate variability derived from a wearable device in accordance with the present invention device may be useful in the diagnosis of illness severity in hypoxic-ischemic encephalopathy infants undergoing therapeutic hypothermia.

Accordingly, several applications are possible with the information that may be gathered and generated with use of a wearable device in accordance with the present invention. As the following figure illustrates other types of information may be derived from sensing data such as the data generated by one or more temperature sensors of the wearable device.

One such example is disclosed with reference to FIG. 5(b), as well as FIG. 5(c) and FIG. 5(d). These figures illustrate exemplary embodiments in which the wearable device includes multiple electrically-conducting contacts, some of which may be placed on the side facing the skin and some others on the other side and exclude temperature sensors so that the wearable device is configured to read and transmit voltage differential data of the wearer.

With regards to the first of these three figures, FIG. 5(b) illustrates a block diagram of a wearable device 510 including multiple electrically-conducting contacts 511, 512, 513, and 514 and does not necessarily include a temperature sensor. Wearable device 510 is as mentioned above configured to generate voltage differentials pertaining to a user, in accordance with exemplary embodiments of the present invention. Specifically, wearable device 500 includes contacts 511-514, which may be coupled to a signal selector, such as a multiplexer, or even as shown multiple multiplexers, mux 1 and mux 2, which receive voltage signals from contacts 511-514, wherein in each mux 1 and mux 2 receive signals from all contacts 511-514. Each mux 1 and mux 2 configured to select pairs of electrodes whose voltages are input to the differential input amplifier 515 and filter 516. A microprocessor 518 may be coupled to the plurality of multiplexers (mux 1 and mux 2), the microprocessor including a memory with a set of executable instructions or logic for generating a plurality of measurements comprising the voltage differences from the voltage values received from each of contacts 511-514. The configuration of the multiplexers in the selection of the contacts may be systematic or may be dynamically driven by the microprocessor, as described later.

In exemplary embodiments, a feedback module such as haptic feedback module 519 may be coupled to microprocessor 518, such that microprocessor 518 is further configured to generate a feedback signal whenever an algorithm of the executable instructions detects an abnormality. For example, and without limiting the scope of the present invention, in exemplary embodiments, when a voltage differential yields a certain value that may be indicative of a medical condition (such as an anomaly in said voltage differentials for the wearer), the microprocessor 518 may be configured to send a feedback signal to a feedback module such as haptic feedback module 519 in order to alert the user.

Exemplarily, haptic feedback module may provide a vibration or other similar feedback that alerts the user to the anomaly. Of course, in other exemplary embodiments, a feedback module may include an audio signal, a visual signal or any combination thereof. Moreover, wearable device 510 may further include a communications module including a transmitter. In such embodiments, microprocessor 518 may be further configured to send a signal to one or more client devices. Moreover, the transmitter may be utilized to send the voltage differential data to the one or more client devices for storage and or further processing.

In exemplary embodiments, device 510 may be placed against a portion of a user's skin such as right below the heart and to the center of the chest area. In such exemplary embodiments, contacts 511 and 512 (may be in direct contact or substantially in contact with a user's skin so that voltage readings from the user's body may be received via said contacts. As such, device 510 may be continuously receiving voltage information. In the event that an anomaly in voltage differences is detected, the user may be prompted with an alert via haptic feedback module 519 to interact or activate the other contacts of the wearable device 510, as will be described below with reference to FIG. 5(c) and FIG. 5(d), as well as with reference to the method depicted in FIG. 5(e).

With reference to FIG. 5(c) and FIG. 5(d), these figures show, respectively, a top and bottom view of another device in accordance with the present invention, wherein a first contact 511 and a second contact 512 may be situated and exposed via a top surface of a housing 510 of the wearable device 500a. Further, a third contact 513 and a fourth contact 514 may be situated and exposed via a bottom surface of housing 510 of wearable device 500a. All such contacts may embody a structure similar to that described with reference to FIG. 4(d). Such wearable device 500a may be worn, for example, on the chest such that contacts 513 and 514 touch the user's skin and such that contact 511 and 512 may be exposed. With this configuration, which facilitates a user to touch each of the top surface contacts 511 and 512, much more meaning full data (other than simple heart beat information) may be generated by wearable device 500a such as echocardiographic data from which meaningful insights and or diagnostic interpretations may be extrapolated.

FIG. 5(e) is a flow chart depicting an exemplary method employed by an exemplary embodiment of the wearable device illustrated in FIG. 5(b)-FIG. 5(c). More specifically, FIG. 5(e) shows method 520 performed by device 510 for providing voltage differential data. It is noted that although method 520 is shown in a particular sequence of steps, other conceivable sequence of the steps may be practiced without deviating from the scope of the present invention.

In step 521, a measurement may be taken from a first contact and a second contact such as contact 511 and contact 512, wherein the measurement comprises the difference between a first voltage V1 and a second voltage V2 (V1-V2) wherein V1 and V2 represent the voltage measured at the first contact and the second contact. In step 512, an algorithm may be performed whereby anomalies may be detected when this differential voltage value (V1-V2) is compared to previously recorded differential values for V1-V2. Such comparisons may include the detection of specific features in (V1-V2), which are then monitored over time. In the event that there is no anomaly, or the voltage differential is within a normal range, then the voltage differential data may be stored and or transmitted to one or more client devices at step 528.

However, if an anomaly is detected, the microprocessor may generate a signal to activate feedback module so that a signal, such as an audio signal, a visual signal, or a haptic signal may be generated. In exemplary embodiments, a haptic signal is generated via haptic feedback module 519. In other exemplary embodiments, an LED is lit up. In other exemplary embodiments, an audio is played via a client device or via the wearable device itself, wherein that embodiment includes a small speaker. In other exemplary embodiments, a notification may be provided to a client device to notify a user of the client device or even the wearer. Whether the anomaly activates a haptic feedback module or any other type of signaling means, the user should be prompted to take action and instructed to activate the other contacts of wearable device 510.

In step 524, accordingly, the user places their fingers over the easily accessible contacts 513 and 514 of device 510. This typically closes a circuit so that additional voltage values of the user's body are received and read by wearable device 510; V3 is the voltage measured at contact 513 and V4 is the voltage measured at contact 514. Reading of voltages from third and fourth contacts may be done by selections in the multiplexers, which many be done on command from the microprocessor.

In step 525, a determination may be made as to whether the user's fingers are properly detected. If not, the user may be continuously reminded via haptic feedback or otherwise, that an anomaly was detected and or that further action is required. In exemplary embodiments, a reminder with specific instructions may be provided to the user via client device such as a smartphone. If the user's fingers are detected by the wearable device, then a next set of measurements may be taken. Such detection may be done by checking if the voltage amplitude measured at the contact exceeds a certain threshold or by detecting specific features in the measured voltage.

In step 526, a series of measurements may be taken from a third contact and a fourth contact such as contact 513 and contact 514, wherein the series of measurements may comprise V1-V3, V2-V4, V3-V4, and V1-V2. When the contacts are used for measuring ECG, the voltage differences may be used to derive voltages from bipolar limb leads, measuring of which facilitates the detection of ailments concerning the heart.

In step 527, a determination may be made as to whether sufficient data has been collected. If enough data is not collected, the user may be prompted to continue or replace their fingers on the contacts 513 and 514 as mentioned above. If enough data has been received, however, then the data may be transmitted to one or more client devices in step 528.

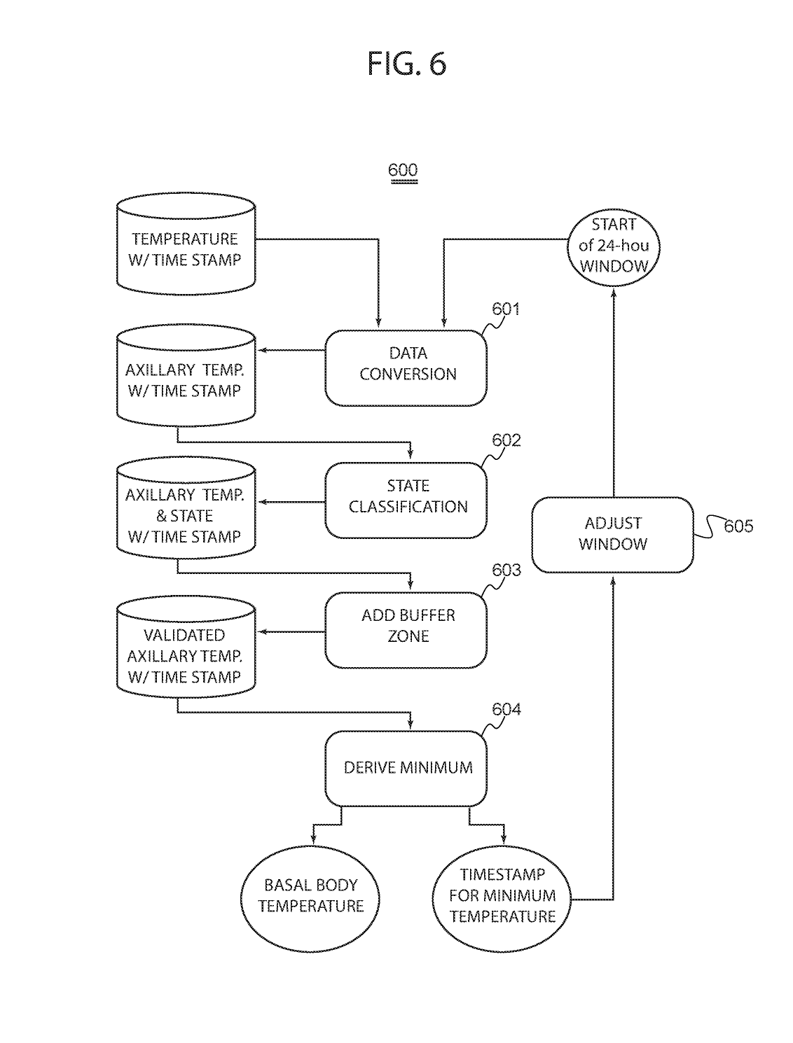

Turning now to the next figure, FIG. 6 illustrates a flow chart of an exemplary method of deriving basal body temperature in exemplary practice of an embodiment of the present invention. More specifically, FIG. 6 shows method 600 for deriving basal body temperature by a client device, with data provided by a wearable device in accordance with the present invention. It is noted that although method 600 is shown in a particular sequence of steps, other conceivable sequence of the steps may be practiced without deviating from the scope of the present invention.

The basal body temperature of a user is the core body temperature when the user is resting. As such, an application in accordance with the present invention (that is for example executable by a client device such as client device 103) may utilize temperature data from a wearable device (for example wearable device 101 that is attached to a user) to compute the basal body temperature for the user for a period of 24 hours.

As illustrated by method 600, data from the wearable device may be first used to compute the instantaneous temperature of the wearer. Starting at step 601, data may be received from the wearable device (including a time stamp).

At step 602, a first check may be run on this data to determine if the device is still worn on a body by rejecting all results that exceed the normal range of human temperature. The result is the combination (body temperature, state of the device) where the state of the device is either on-body or off-body. All temperature data in off-body state may be ignored.

At step 603, for all transitions from on-body to off-body and vice-versa, a buffer zone may be created so that temperature data in this buffer zone may be ignore for calculations of basal body temperature.

Then, at step 604, all data that is not ignored may be run through a short-term averaging filter to compensate for noisy measurements, duly tracking the breaks in the data stream. The lowest measured temperature in the averages provide the basal body temperature.

At step 605, the timestamp associated with basal body temperature may then be used to refine the 24-hour period for future measurements. One way to adjust the 24-hour period may be to move the time associated with the basal body temperature to the middle of the 24-hour period. Optionally, the window may be moved by a few minutes per day instead of moving the window abruptly.

The next set of figures disclose and or illustrate how data from the wearable device may be utilized for deriving useful insights pertaining to the health of the user. Primarily, the next figure illustrates how a wearable device state may be determined, which as shown in the discussion with method 600 is useful information for calculating measurements such as basal body temperature.

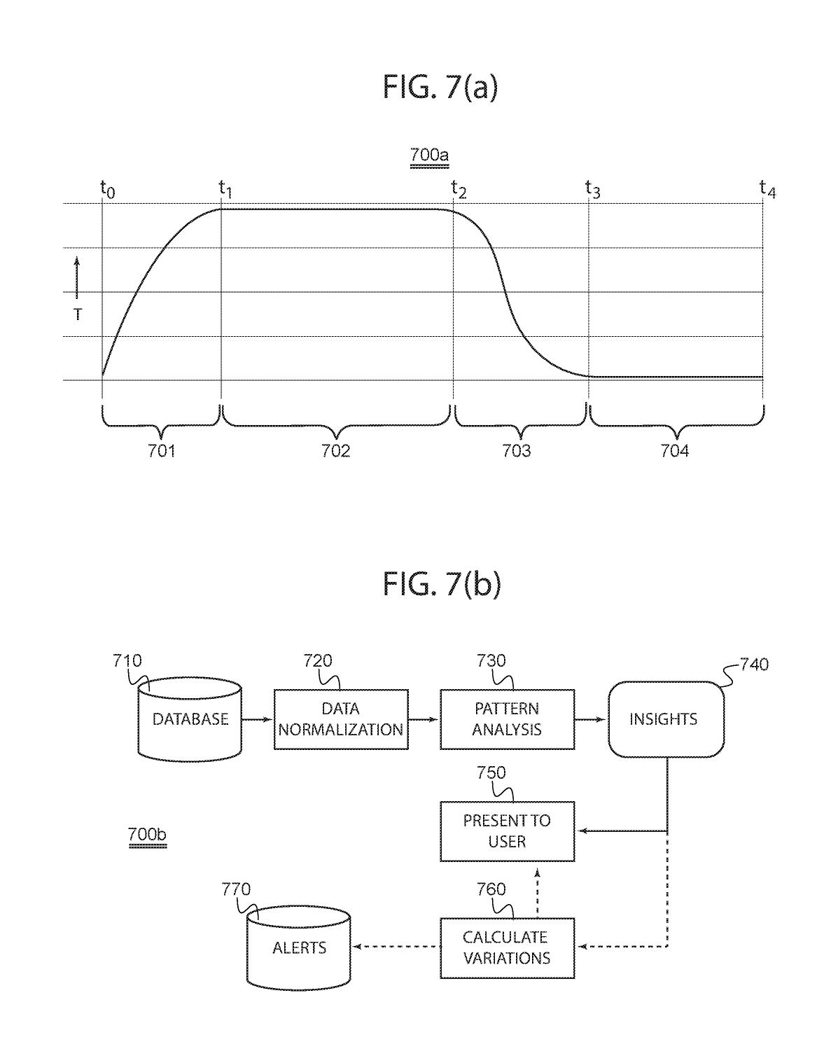

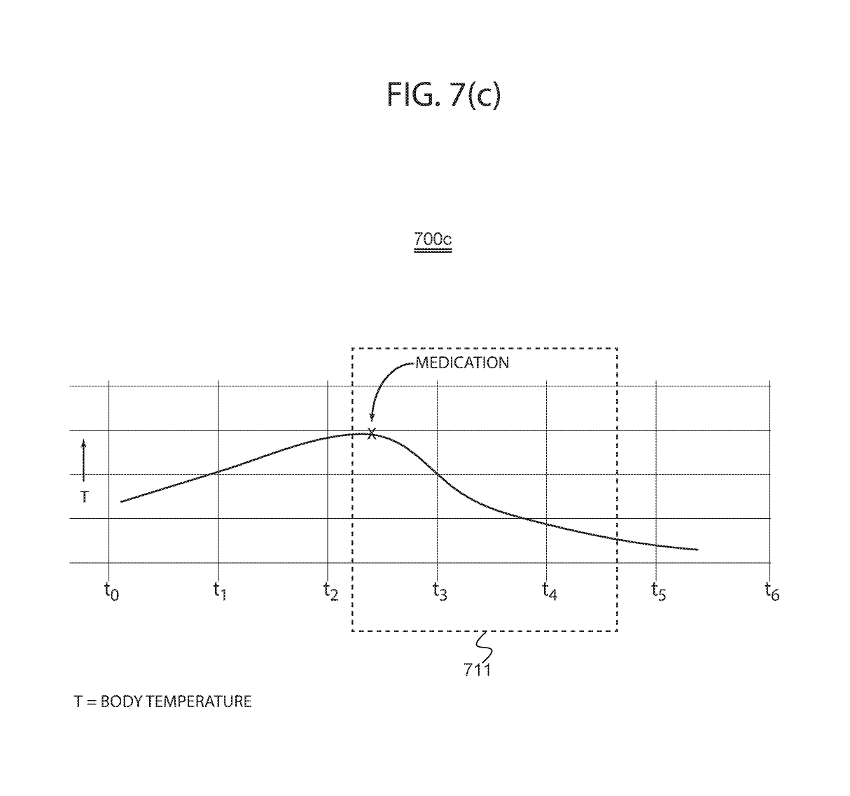

Turning now to the next set of figures, FIG. 7(a) is a chart showing temperature as a function of time, which demonstrates a method for determining different device states in exemplary practice of an embodiment of the present invention. More specifically, FIG. 7(a) depicts chart 700a showing a curve representing temperature (T) as a function of time (t) throughout arbitrary periods of time (pₓ). Again, this is merely illustrative should not limit the scope of the present invention. More importantly, this information may be used, for example, by an application—such as application 103a—to make certain determinations utilizing a state of the wearable device.

For example, and in no way limiting the scope of the present invention, as may be appreciated from chart 700a, between periods t₀ and t₁ the temperature curve is converging, showing the temperature of the wearer increasing. As such, every time temperature data reveals a continuously rise in temperature, a first type of device state 701 may be assigned. This state may be indicative of the device recently being attached to the skin of a user or initially worn; alternatively, this may indicate a rise in the temperature of the wearer. Naturally, a converging state or state 701 may have different implications depending on the condition of the user, such as an abnormal increase in temperature indicative of a serious or worsening condition, an expected increase in temperature as a result of applied heat, or as stated above, merely that the device has been recently worn after nonuse. Accordingly, a first type of device state 701 may be a converging state.

Similarly, as may be appreciated from chart 700a, between periods t₁ and t₂ the temperature curve is steady, showing the temperature of the wearer constant. As such, every time temperature data reveals a constant temperature, a second type of device state 702 may be assigned. This state may be indicative of normal or steady (indicating that the device is being worn, although not necessarily indicating a normal temperature of the user—if for example the user is a patient with a condition where a low or high temperature is expected during a period of time).

Similarly, as may be appreciated from chart 700a, between periods t₂ and t₃ the temperature curve drastically falls, showing the temperature detected by the device decrease severely. As such, every time temperature data reveals a serious decrease in temperature, a third type of device state 703 may be assigned. Although this state may be indicative of a severe condition, it is most likely indicative of the device either falling off, or simply not being worn properly. This information may be useful, for example, to: generate instructions concerning an alert that the device is not properly attached to the wearer, generate instructions concerning notifications that the device will power down, generate instructions concerning power consumption; or useful in certain determinations wherein certain temperature ranges must be ignored as discussed above in reference to method 600.

Finally, as may be appreciated from chart 700a, between periods t₃ and t₄ the temperature curve is steadily at a nil temperature reading. As such, every time temperature data reveals this state, a fourth type of device state 704 may be assigned to indicate the device has been removed or turned off.

Each device state may be used by the wearable device's programmable instructions as well as an application on a client device in communication or with access to the data from the wearable device. For example, and without limiting the scope of the present invention, when firmware in the device discovers that it is presently in state 704 wherein the body that it is connected to is well below the human body temperature, the device may conclude that the device has fallen of the body that it was measuring. In that situation, the device may inform the temperature and the state of the device to the application and after a certain time has elapsed, it goes into shutoff mode.

Moreover, device states may be optionally controlled by a user. For example, and without deviating from the present invention, a user may manually put the wearable device in either state 703 or state 704. A user may initiate a state 703, for example, by enabling a type of ‘airplane mode’ by making a selection in a client device's application in communication with the wearable device; in airplane mode, the processor/RF transmitter power may be disconnected preventing inadvertent data transmission and power consumption but may nevertheless still actively receive sensor readings. Alternatively, the user may initiate state 704 by pressing an input button on the wearable device for an extended period (e.g. more than 7 seconds) to simply turn the device off. Of course, these are merely examples and a person of ordinary skill in the art will appreciate that other configurations are possible, including enabling airplane made from the wearable device itself or the application, as well as turning the device off completely from the application or from the wearable device. In an exemplary embodiment, the wearable device has a button that enables airplane mode and allows the device to be turned off.

One key feature of an application in accordance with the present invention is detecting patterns in user data so that insights can be drawn and presented to users including the user-wearer or authorized users. Some of these insights may be used in detecting drug efficacy and user adherence to prescription. Insights may also be combined with other databases to suggest a diagnosis for the symptoms detected. Moreover, such insights may be utilized for generating alerts that automatically notify users of relevant information. The next figure references one exemplary method for providing insights.

FIG. 7(b) is a flow chart depicting an exemplary method of deriving and presenting insights to a user in exemplary practice of an embodiment of the present invention. More specifically, FIG. 7(b) shows method 700b; it is noted that although method 700b is shown in a particular sequence of steps, other conceivable sequence of the steps may be practiced without deviating from the scope of the present invention. In exemplary embodiments, an application is configured to generate and present specific insights to a user, for example a user of client device 103 or client device 104.

As illustrated by method 700b, typically, a device such as client device 103 or client device 104 may receive data from the wearable device such as device 101 at step 710. As mentioned above, this may comprise communicating with the device directly or receiving data that was previously stored at a remote location such as at server 106.

In step 720, an application may detect patterns in user data and as a first check, the data from the wearable device may be normalized based on long-term averages and ranges.

In step 730, the normalized data may be subject to pattern analysis using algorithms such as singular value decomposition or Fourier transform or cross-correlation.

In step 740, the analysis of the resulting transformation may be utilized to produce insights.

Importantly, although these steps may be performed by, for example, an application situated at a client device, such as application 103a of client device 103, in some embodiments, insights may be generated remotely, for example by software situated at server 106.

In either scenario, at step 750, insights generated by one or more programmable instructions situated on a client device or server may be presented to the user. An example use of the core body temperature is to detect if the wearer of the wearable device is suffering from parasitic fever. An example use of body temperature and voltage difference is to measure concurrently the body temperature and heart rate, which can be used to measure calorific output or identify stress and its cause.

Optionally, at step 760, the analysis may also track variations in the patterns of user data. An example application is in the detection of heart rate variability, which can be used for identifying Hypoxic-Ischemic Encephalopathy in children.

Optionally, at step 770, when there exists a baseline, variations from the baseline may be delivered to the user as alerts.

In an exemplary embodiment, insights detected by an application in accordance with the present invention may be presented to a user in the form of a graph with time as the x-axis variable. FIG. 7(c) is a chart showing temperature as a function of time, which demonstrates such example. For example, and without limiting the scope of the present invention, measured body temperature may be displayed in a first color for normal and excursions outside the normal range may be displayed in a second color. Insights may also be derived using user input concerning certain events.

In exemplary embodiments, a graphical display of measurements (such as those displayed by the curve of graph 700c) may be supplemented by an overlay of notifications that arise from user actions such as the application of a certain medication. The end-result is a single view that shows measurements and user actions. An application of this method is in determining the effect of a medication on user's health as monitored by the wearable device. For example, a user may indicate via an input on their smartphone that they took a certain fever reducing medication shortly after time t₂ (in graph 700c). With this information, the application may track the effects of the medication during a period set 711 to provide feedback on things like, the effectiveness of the medication, whether a lower or higher dosage is required, etc. Optionally, the application may transmit the data stored locally to a server that hosts the data. Optionally, the application enables a user to enter reminders that notify the user when the timing is appropriate for a specific action, such as taking a new dose of medication.

Accordingly, an application in accordance with the present invention may be configured to display real-time information on a smartphone or tablet. Data may be stored internally (or locally) on a client device, and produce useful outputs such as graph 700c. In exemplary embodiments, the application providing insights may be kept up to date by using synchronization methods, or seeking information it has not previously received from the wearable device at regular or predetermined intervals. Several uses for and types of insights are disclosed below by way of illustration and are in no way intended to limit the scope of the present invention.

Estimation of Medicinal Cause and Effect: In some exemplary embodiments, an application in accordance with the present invention may be configured to determine whether the medication prescribed by the physician is taking effect. In such method, a user may be asked to enter into the application certain information such as the medication that is prescribed and the timings of reminders that the user desires (or presumably have been prescribed). The application may thus remind a user at the appropriate time and monitor the user's measurements to detect any response. One example may include the response to the administration of an antibiotic for a patient with a bacterial fever, which may be tracked by means of a reduction in the body fever. If no change is observed, then an alert is created for the medical provider.

Medicine Adherence: In some exemplary embodiments, an application in accordance with the present invention may be configured to use the patterns detected in the body temperature along with the knowledge of local epidemics to recommend a diagnosis to the patient's medical provider.

Ovulation Monitoring for Conception and Contraception: In exemplary embodiments, a specific insight that may be presented to the user is a fertility score. The application may implement known technics but utilize the basal body temperature derived using the method described above with reference to FIG. 6 in order to estimate the fertile days for a woman.

Fever Compensation for Ovulation Monitoring: In some exemplary embodiments, an application in accordance with the present invention may be configured for fertility tracking; in such embodiments, the application may compensate for fever for a more accurate prediction of a woman's fertility. Fever is detected as an elevation in the temperature measurements made by the wearable device. This elevation is measured and is compensated from the temperature measurements before it is used in the calculation of basal body temperature. The resulting basal body temperature is then used in the estimation of fertility score.

As such, these and many other methods maybe implemented utilizing information gathered by a wearable device in accordance with the present invention. Although, as mentioned above, it is possible for a wearable device to be configured for performing many of the above-discussed calculations, typically these calculations are left to more processor intensive devices such as client device 103, in order to keep the wearable device as battery efficient as possible.

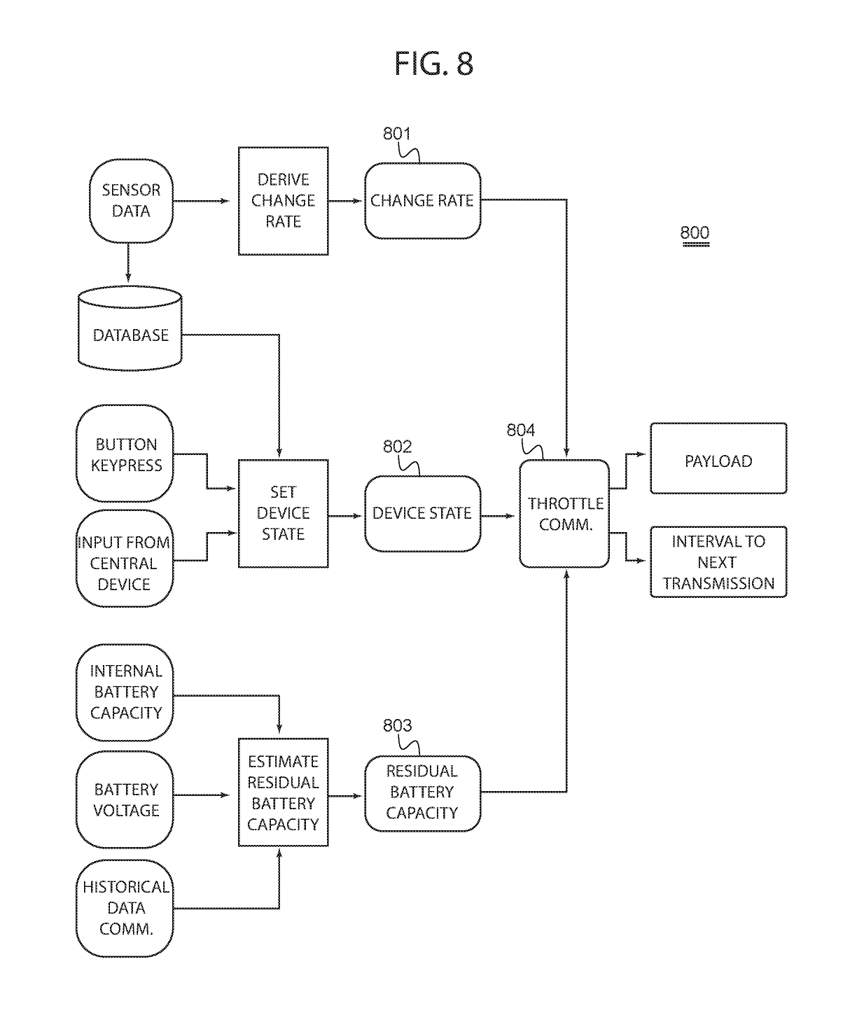

Moreover, as will be discussed in turn with reference to FIG. 8, a wearable device in accordance with the present invention may implement algorithms to improve battery life. For example, in exemplary embodiments, communications from the wearable device to a client device may be throttled to by using a combination of several factors including rate of change in data, residual battery capacity and prior data transfers.

Communication is a key driver for power consumption. In order to minimize power consumption, methods exist to reduce the amount of communication based on the amount of data to be transmitted. Some methods use known techniques to reduce power consumed during transmission by using multiple different advertising packets or in different operating modes. However, that method does not take into consideration the amount of battery capacity left in the wearable device. In the present invention, the time interval between transmissions and the format for the payload may be derived from the amount of data to be transmitted, operating mode, and the residual battery capacity. FIG. 8 is a flow chart depicting an exemplary method of throttling data in exemplary practice of an embodiment of the present invention. More specifically, FIG. 8 shows method 800; it is noted that although method 800 is shown in a particular sequence of steps, other conceivable sequence of the steps may be practiced without deviating from the scope of the present invention.

In step 801, sensor data may be initially used to compute the rate of change of data.

In step 802, the most recent sensor data, in combination with near-term history, may be used to determine the state of the device (as discussed above). Optionally, the state of the device may be set by other means, such as by the press of the button on the device or by a directive from the central device such as a phone.

In step 803, a residual battery capacity estimator may implement measurements of battery voltage, duration of active use and amount of data communicated to estimate the amount of battery capacity left over.

In step 804, the rate of change of data determined in step 801, the device state determined in step 802, and residual battery capacity determined in step 803 may be used by means of a linear equation to determine the payload format and the duration of the time interval to the next transmission.

It is well-known that the estimation of battery capacity using the voltage of the battery is not accurate because of the current drawn from the battery previously and variations from one sample of the battery to another. Accordingly, in exemplary embodiments, estimating the residual capacity of the battery using the time the wearable device has been in active operation, and the amount of data transmitted by it, may be implemented. Active operation may only include the time that the processor has been powered up.

In such exemplary method, the wearable device tracks the time of active operation and the number of data blocks transmitted. Using these values as independent variables, the wearable device uses a linear equation to estimate residual battery capacity. The coefficients for the linear equation are provided by the application in the client device during the provisioning process. The wearable device may execute this method either periodically or when a block of data is transmitted or both.

Of course, a person of ordinary skill in the art will appreciate that other methods may be implemented to maximize battery efficiency. For example, one method of increasing battery life may simply include detection of when the device is not being used in order to automatically shut it down, for example as mentioned above by detecting the state of the device.

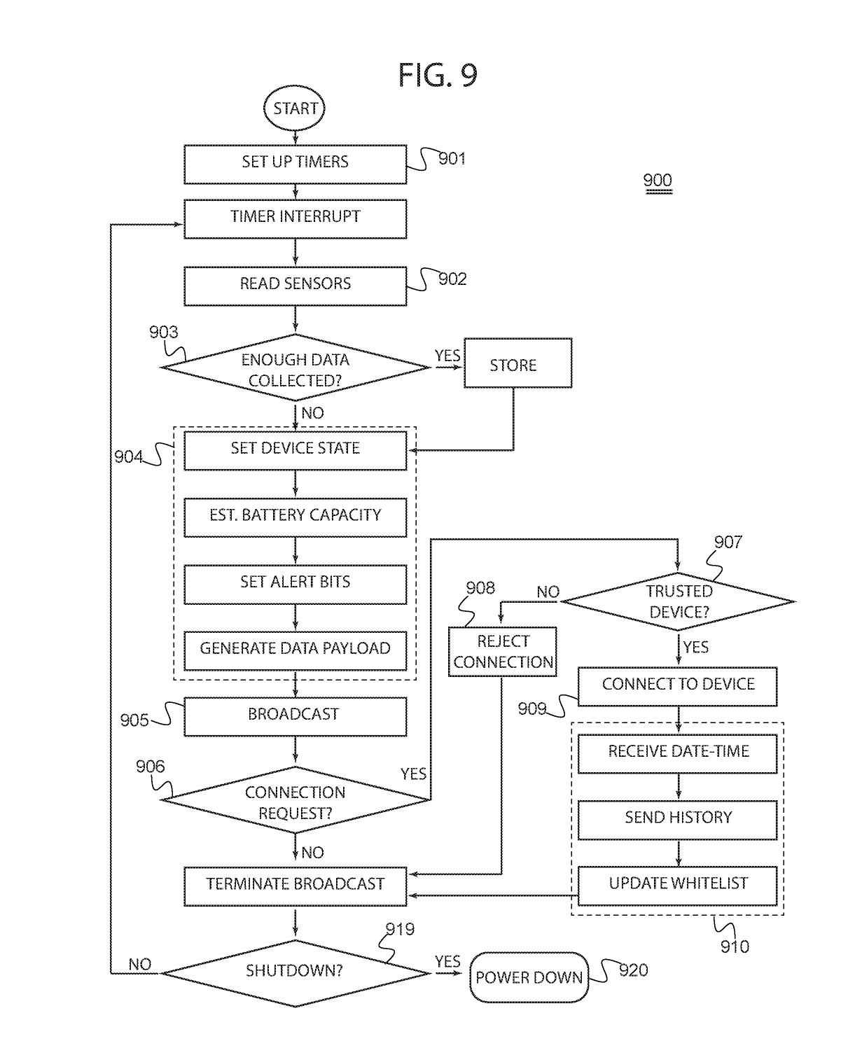

Turning now to the next figure, FIG. 9 is a flow chart depicting an exemplary processing flow of typical programmable instructions on a wearable device in accordance with the present invention. More specifically, FIG. 9 shows method 900; it is noted that although method 900 is shown in a particular sequence of steps, other conceivable sequence of the steps may be practiced without deviating from the scope of the present invention.

In steps 901-902, measurements of the temperature and optionally, the voltage difference and/or the accelerometer may be received to generate the body's core temperature, the heart rate, heart rate variability, the respiration rate and sudden motion vector that comprise the data payload.

In step 903, a determination may be made as to whether enough data has been collected. If sufficient data has been collected (e.g. an hour's worth of data), then this information may be computed and or stored in the wearable device's memory, for example in a local non-volatile RAM.

In step 904, device state information may be derived from the computed data. For a device only containing the temperature sensor, the state could be converging, steady-state on body or steady-state off-body. Payload is derived from the data computed. Alerting bits in the payload are set based on the values of the data computed. For a device only containing the temperature sensor, the alerting bits that the core body temperature computed has exceeded normal range of human body temperature. Residual battery capacity is estimated and is used to schedule the next transmit time.

To transmit data, the wearable device starts broadcasting at step 905. If it is unable to verify that the requesting client device is a trusted device, then via steps 906-908, the connection may be rejected. If the requesting client device is a trusted device and the wearable device is able to authenticate the client device, then at step 909, the device may receive a request for establishing a communication.

In steps 910, the client device may want to respond based on the data received. In some embodiments, if this data is from a trusted device, the wearable device connects securely to this trusted device and either receives current date and time, sends history or updates whitelist.

When the connection is terminated, the wearable device completes advertising. If requested to shut down, at step 911, the wearable device may shut down. Otherwise, a goes lower power mode may be initiated waiting for an interrupt to occur.

Accordingly, an important aspect of the present invention is in the security protocols used for onboarding the device, that is, in putting the device into use and periodically in communication with one or more client devices. To these ends, the present invention may implement methods such as geo-proximity and passkey security to ensure minimization of intrusion while a secure connection is established for sharing of encryption keys for subsequent communication. Using these methods, user data may be encrypted at the application level to offer security from eavesdroppers as well as malicious users that may gain access to a client device trusted by the wearable device. The next figure references one exemplary approach for enabling secured communications with a client device.

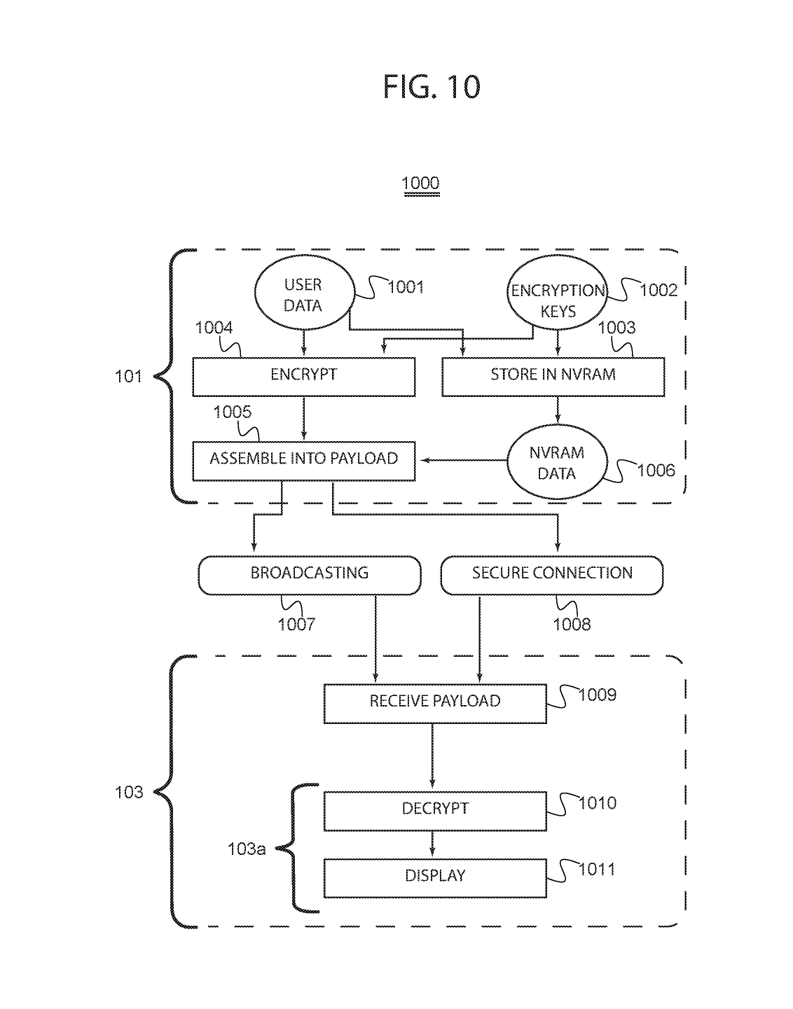

FIG. 10 is a flow chart depicting an exemplary method of communicating user data between a wearable device and a client device in exemplary practice of an embodiment of the present invention. More specifically, FIG. 10 shows transmission of user data from wearable device 101 to client device 103 running application 103a. Irrespective of the channel used for transmission, the wearable device encrypts user data at step 1002 based on the encryption keys that wearable device 101 received during onboarding at a previous step 1001. Application 103a typically decrypts encrypted data if it knows the encryption key, which happens only if client device 103 running application 103a is authorized to receive that information. The scheme for encryption may be used even when user data is sent over a secured connection so that means are provided to application 103a to secure user data from a malicious user who gets access to client device 103 but is unable to gain access to application 103a.

In an exemplary embodiment, user data in a wearable device may be stored in an encrypted form. If the wearable device is for some reason compromised, this condition may be detected by the device's self-test. In this mode, the user data may be rendered unusable by for example, erasing the encryption key upon failure of the self-test, as described above.

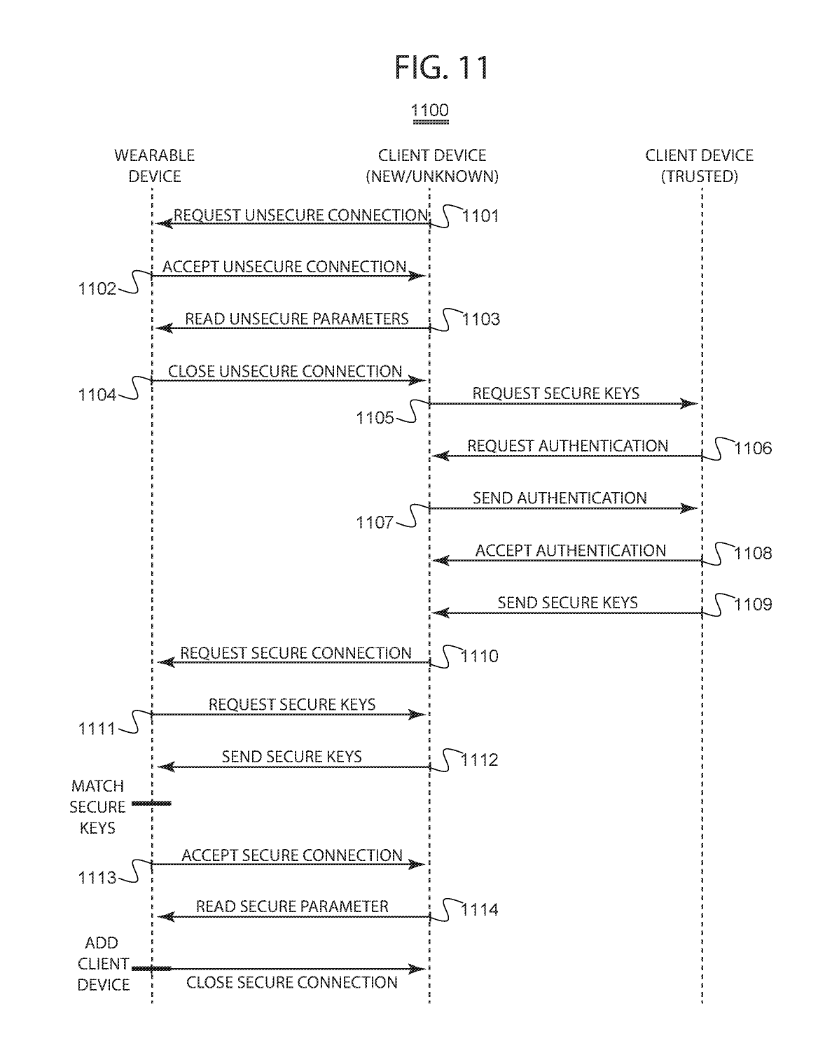

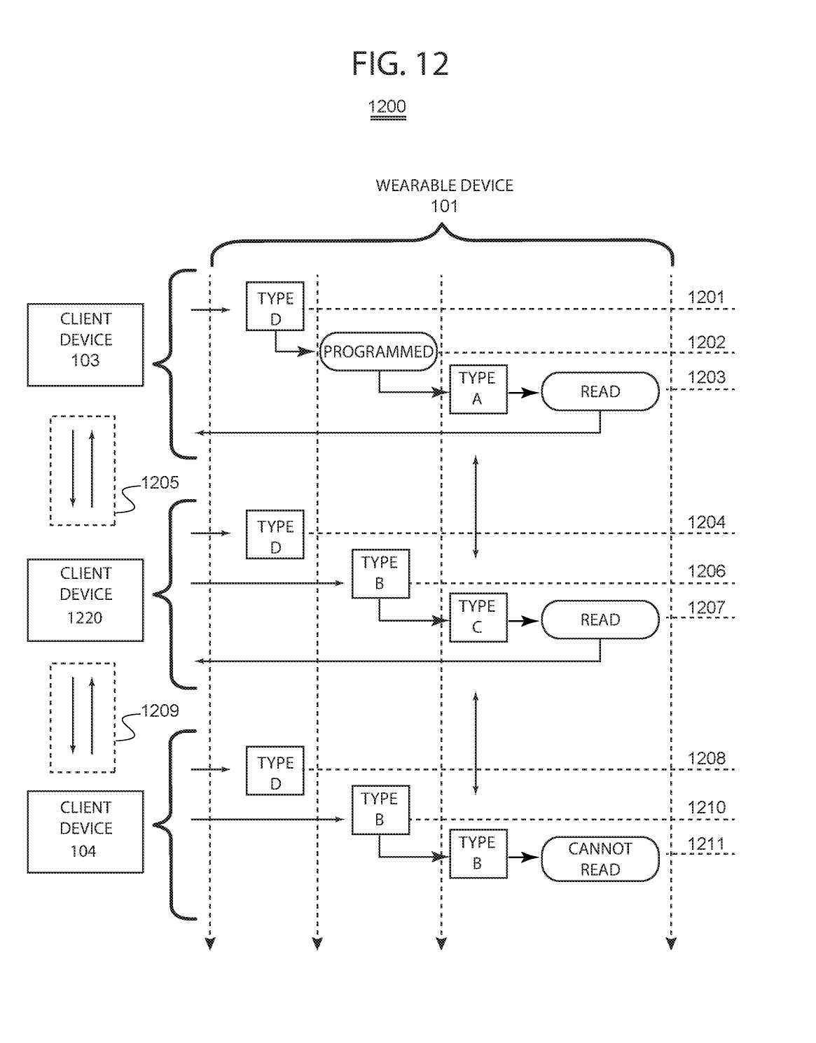

Turning now to FIG. 11, a flow chart is illustrated depicting an exemplary method of authorizing one or more devices to read data captured by a wearable device in exemplary practice of the present invention. More specifically, FIG. 11 shows method 1100; it is noted that although method 1100 is shown in a particular sequence of steps, other conceivable sequence of the steps may be practiced without deviating from the scope of the present invention.

In this exemplary method, a wearable device may store identifying information concerning previously trusted client devices that have connected to the wearable device securely. This identifying information may be used to limit acceptance of connections from devices it has connected to. With reference to FIG. 11, previously connected devices may be referred to as “trusted devices.” As such, in exemplary embodiments, a wearable device may store a list containing identification numbers or otherwise identifiers for several trusted devices. A trusted device may then pass on the trust credentials to a secondary device so that it too can connect to the wearable device and get added to the list of trusted devices. When the wearable device is provisioned for use by the user, a client device not involved in the provisioning process (i.e. is not automatically added to a trusted device list) may use the following process to obtain authorization to access the wearable device.

In step 1101, a new client device may request to connect to a wearable device such as wearable device 101. In step 1102, wearable device 101 may accept a limited unsecured connection to the new client device. Because the connection is unsecured, at step 1103 the new client device may read unsecured parameters provided by wearable device 101. Typically, not being a trusted device, wearable device 101 will close the unsecured connection with the new client device since the new client device is not recognized for communication of user data.

In these instances, the new client device must obtain permission from the user of wearable device 101 to communicate with the wearable device. One way to achieve this is to request authorization from a previously provisioned device such as a trusted client device, wherein authorization comprises providing security keys from the trusted device to the new client device. In exemplary embodiments, a matrix code or other similar means may be implemented to obtain secured keys that were issued to the trusted client device.

Accordingly, in step 1105, the new client device may request security keys. In step 1106, the trusted client device may further request some type of authentication such as a passcode that may be provided by a user of the trusted client device to the user of the new client device in step 1107. In some exemplary embodiments, utilizing a typical matrix code may obviate these last steps. The transfer of keys between devices may also be achieved over a secure connection, such as over a BLE connection. Optionally, the keys may be transferred using a server computer in the cloud.

In steps 1108-1109, whatever method may have been implemented, typically results in the trusted client device providing the secured keys to the requesting new client device.

Accordingly, now the new client device will be recognized by wearable device 101 when requesting a secured connection at step 1110; and at step 1111, in response to a request by wearable device 101 for secured keys, the new client device may provide secure keys obtained from the trusted client device in previous steps.

Upon receipt of secured keys, having matched to encryption keys stored in a memory of wearable device 101, a secured connection may be established at step 1113. In exemplary embodiments, secure or encryption keys may be changed periodically by the wearable device and communicated to trusted devices in communication range.

At step 1114, the new client device may access user data transmitted by wearable device 101.

As a convenience measure, in exemplary embodiments, the new device may be added to a list of trusted devices. In other exemplary embodiments, wearable device 101 may further categorize the new device in a method that adds further security in the manner that wearable device 101 and authorized client devices communicate. Such method is discussed in turn.

FIG. 12 is a flow chart depicting an exemplary method of authorizing one or more devices to read data captured by a wearable device in exemplary practice of an embodiment of the present invention. More specifically, FIG. 12 is a flow chart depicting method 1200, in which client device 103 is provisioned to communicate with wearable device 101, and provides authorization to a new client device 1220 to communicate and read data from wearable device 101. With method 1200, multiple trusted client devices may access user data when they come in range of wearable device 101; this includes the ability for trusted devices such as smartphones to seek historical data from the wearable device when the wearable device comes in range.

In an exemplary embodiment, wearable device 101 may categorize client devices in order to ensure that proper user permissions have been established. Such categories may be labeled for illustrative purposes as Type A, Type B, Type C, and Type D devices. Without limiting the scope of the present invention: a Type A device may refer to a device that has been previously provisioned to communicate with wearable device 101; a Type B device may refer to a device that has been previously provided with encryption keys; a Type C device may refer to a device that has been previously provided with encryption keys by some other application (not a Type A device) but has been granted access to wearable device 101; and a Type D device may refer to a device that has not been provisioned to communicate with wearable device 101. In exemplary embodiments, wearable device 101 typically scans for devices filtered by a specific manufacturer ID or other identification, and determines whether to allow that discovered device to read user data, depending on the category or type of device discovered.

Accordingly, assuming client device 103 has not previously been provisioned with wearable device 101, in step 1201, when client device 103 seeks to communicate with wearable device 101, wearable device 101 will see the device as a Type D device since client device is not yet in its database of previously discovered devices. At this step, wearable device 101 may initiate a provisioning protocol. In exemplary embodiments, an application on client device 103 may query the user whether the user wants to put client device 103 into use. If the user wants to use this device, the user may be required to enter a passkey or passcode granting secure access to wearable device 101. If the user is unable to provide this passkey, then the user does not get secure access to the device and is therefore, not able to provision the device. As discussed before, the application allows the user to enter the passkey using several means such as manual entry, a visual scan of a static phrase or a visual scan of a blinking LED.

In step 1202, assuming wearable device 101 and client device 103 share the same user, for example, then presumably the user of client device 103 has the required passcode and is able to provision or program client device (for the first time) to communicate with wearable device 101.

In step 1203, because client device 103 has been provisioned, wearable device 101 categorizes client device 103 as a Type A device for future pairing. Moving forward, client device 103 is able to retrieve in real-time the user data captured by wearable device 101, including measurements that may be converted to graphs, tables or any form that is meaningful to the user via a display of client device 103 available for processes the data for further actions.

In step 1204, supposing that another device is within range of wearable device 101, for example client device 1220, then wearable device 101 will see client device 1220 as a Type D device. Assuming client device 1220 is not, and will not be, provisioned (for example, it is a device belonging to a different user than the user of client device 103 and wearable device 101), then client device 1220 will not be allowed to read user data.

However, the user of client device 1220 may approach the user of client device 103 and if desired, the user of client device 103 may provide authorization (and hence encryption keys) to access data on wearable device 101. As mentioned with reference to FIG. 11, this may be achieved via numerous methods, and in an exemplary embodiment a matrix code may be used to share the encryption keys between client device 103 and client device 1220 at step 1205. As mentioned above, this may be desirable for example when a family member or caregiver may need access to user data of wearable device 101.

Accordingly, after receiving the encryption keys in step 1205, in step 1206, wearable device 101 will discover client device 1220 as see it as a Type B device since client device 1220 has been previously provided with encryption keys. In this step 1206, wearable device 101 may obtain identifying information to seek whether this device has been provided encryption keys by a proper authorizing party such as a Type A device.

In step 1207, since client device 1220 was provided encryption keys from a Type A device (client device 103), then wearable device 101 categorizes client device 1220 as a Type C device and allows it to read its data. Again, this may be achieved by various means including for example reading a device ID identifying client device 103 as the source of the encryption keys. Moving forward, client device 1220 is able to retrieve in real-time the user data captured by wearable device 101, including measurements that may be converted to graphs, tables or any form that is meaningful to the user via a display of client device 103 available for processes the data for further actions. Of course, if the encryption keys are changed—as they may be periodically, then client device 1220 may be required to obtain new encryption keys following a similar method.

In step 1208, a new device—for illustrative purposes client device 104—may now try to read user data from wearable device 101. Again, wearable device 101 will see client device 104 as a Type D device. Assuming client device 104 is not, and will not be, provisioned (for example, it is a device belonging to a different user than the user of client device 103 and wearable device 101), then client device 104 will not be allowed to read user data.

As a measure of security, if at step 1209, the user of client device 104 approaches the user of client device 1220 for authorization (and hence encryption keys) to access data on wearable device 101, these keys will not be valid. This is because even though client device 1220 may have shared encryption keys with client device 104, in step 1210, wearable device 101 will see that it is a Type B device, and when seeking an identification of the source of the encryption keys, will not see a Type A as the source.

From the perspective of wearable device 101, since client device 104 is neither a Type A or a Type C, client device 104 will not be granted access to read user data from wearable device 101. Accordingly, an application on client device 104 is unable to retrieve the measurements made by wearable device 101; if the application desires access to this device, then it must seek secure keys from an application that has access to the device, as explained in the above—for example from client device 103.

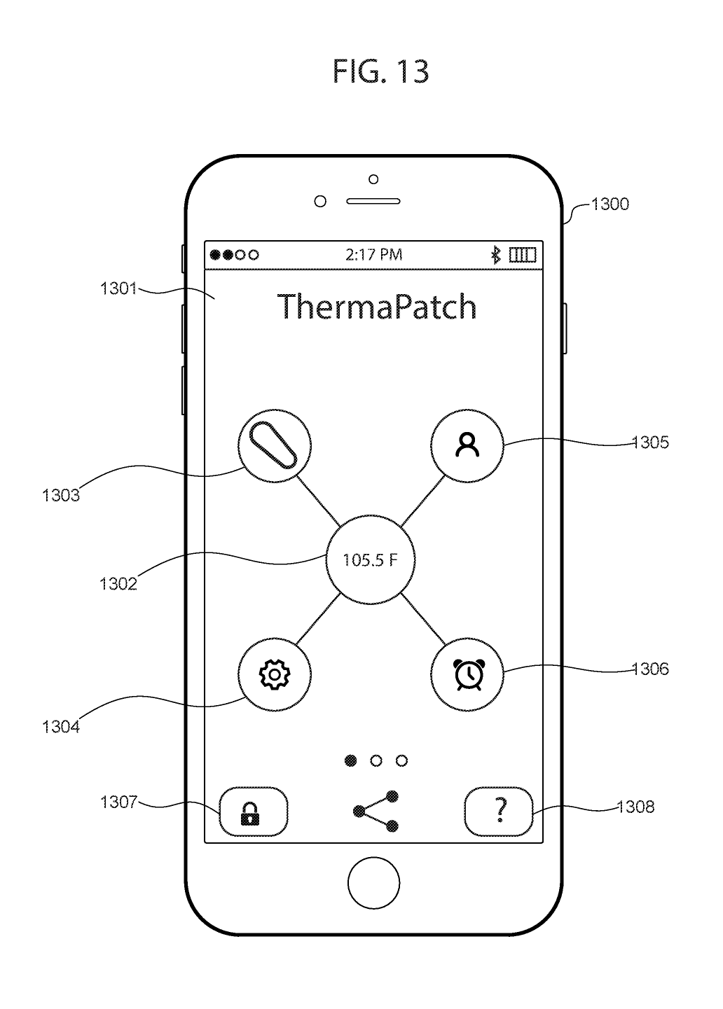

Turning now to the last set of figures, FIG. 13-FIG. 29 illustrate an exemplary user interface such as a graphical user interface (GUI) that may be distributed to a plurality of client devices in accordance with exemplary embodiments of the present invention. More specifically, these figures depict an exemplary client device 1300 such as a common smartphone that displays a user interface or mobile device application with different output displays that facilitate monitoring the health data that may be continuously provided by wearable devices in accordance with the present invention. In some embodiments of the present invention, a wearable device may include a thermal sensor for providing temperature data of the wearer to one or more users with access to client device 1300. In other exemplary embodiments, the same user or a different user may further utilize a wearable device configured to generate voltage differential data of a wearer of the device. As such, these users or user may review and thus monitor their health by visualizing their data and being provided with some meaningful insights as well as functions that help the wearer or user to monitor their health.

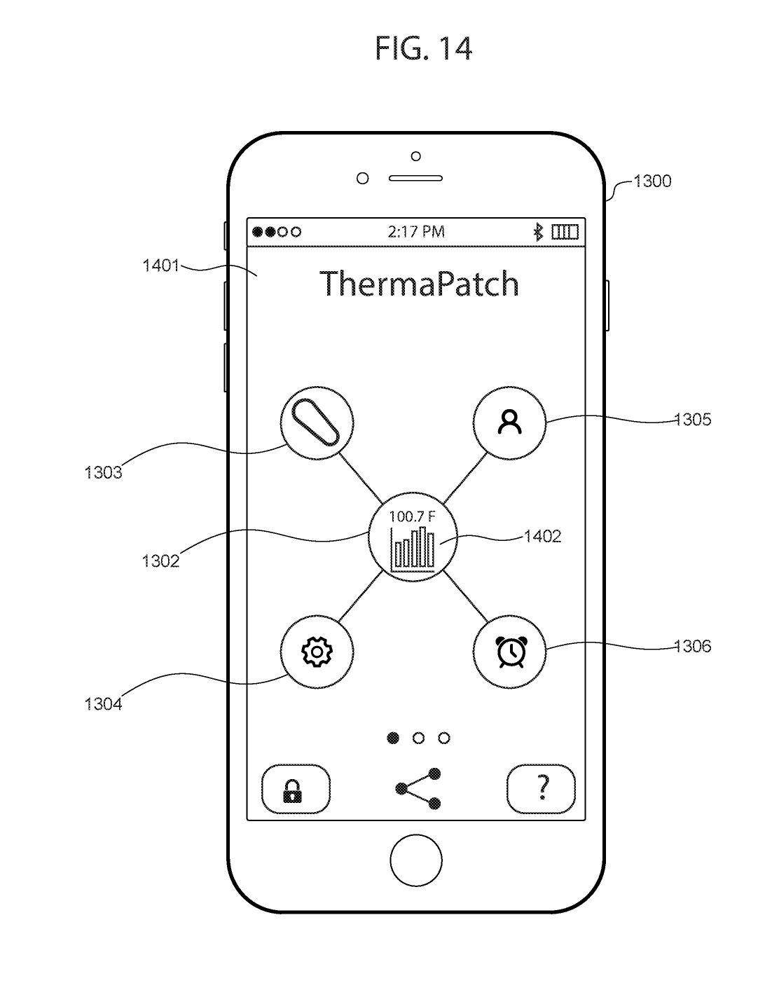

With reference to FIG. 13 and FIG. 14, output displays 1301 and 1401 are very similar. In the former, a display may exemplarily include certain indicators that provide immediate information to a user such as indicator 1302, which includes the wearer's temperature. Other indicators may include indicator 1303 that may display the type of device being detected and from which client device 1300 is receiving the health data. For example, in the instant case an icon representative of a wearable temperature sensing device may be displayed. Indicator 1304 may be a data object such as a button to access certain settings of the mobile application as will be discussed further below. Indicator 1305 may be a similar button that provides access to a user management functionality for adding users and or associating users with a particular wearable device. Indicator 1306 may include an alarm icon or the like and may provide access to a reminder settings functionality to add, set, activate or disable reminders for different items such as medications, temperature readings, etc. as will discussed further below. Of course, a person of ordinary skill in the art will appreciate that other functionalities may be provided without deviating from the scope of the present invention, such as security measures via security input to prevent settings from being changed via indicator 1307, or a help feature for providing guidance to a user via help indicator 1308. Specifically, with regards to FIG. 14, display output 1401 illustrates that naturally more or less complex information may be provided to a user, such as, for example, a temperature reading along with a graphical representation 1402 of a particular trend.

Some of the functionalities that may be provided via such an application to users of wearable devices in accordance with the present invention, may include visual and or functional prompts that guide the user to associate their wearable device with a particular client device. These functionalities not only enable a first time user to easily associate their device to their smartphone, but also ensures that their health data is kept secured.

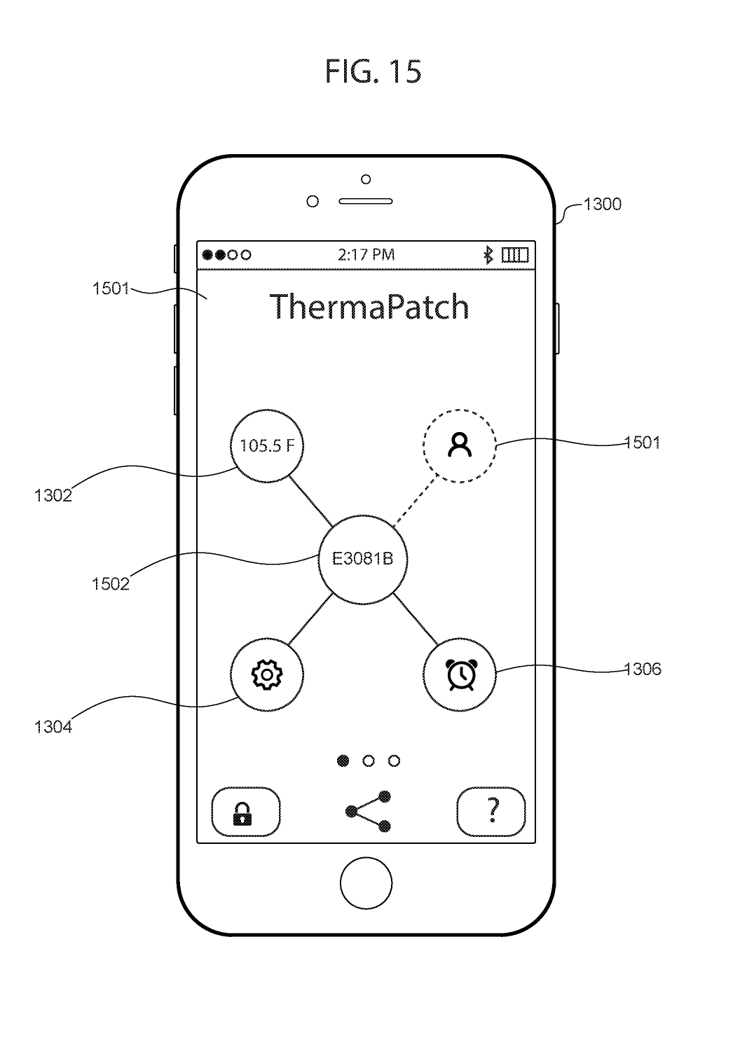

Now with reference to FIG. 15-FIG. 20, FIG. 15 depicts display output 1501, which may include a blinking indicator 1501 guiding the user to select said indicator so that they may add a user of their wearable device to the client device. That is, typically upon first use, a wearable device (for example having a thermal sensor) configured to provide temperature data, may initially allow for some communication of the temperature data while the device is paired with the client device 1300. As such, a current temperature may always be displayed for a few initial minutes. However, because this may be the first time the device is set up, a centered indicator may display an ID associated with the wearable device. As mentioned above, an application or GUI for users of wearable devices in accordance with the present invention, may include visual and or functional prompts that guide the users to certain actions, including providing a limited set of actionable data objects, blinking data objects, or other types of prompts. For example, and without deviating from the scope of the present invention, because indicator 1501 is blinking, or for example may be the only enabled indicator available to the user, the user will be guided to select indicator 1501 to add and or associate their wearable device to a particular user.

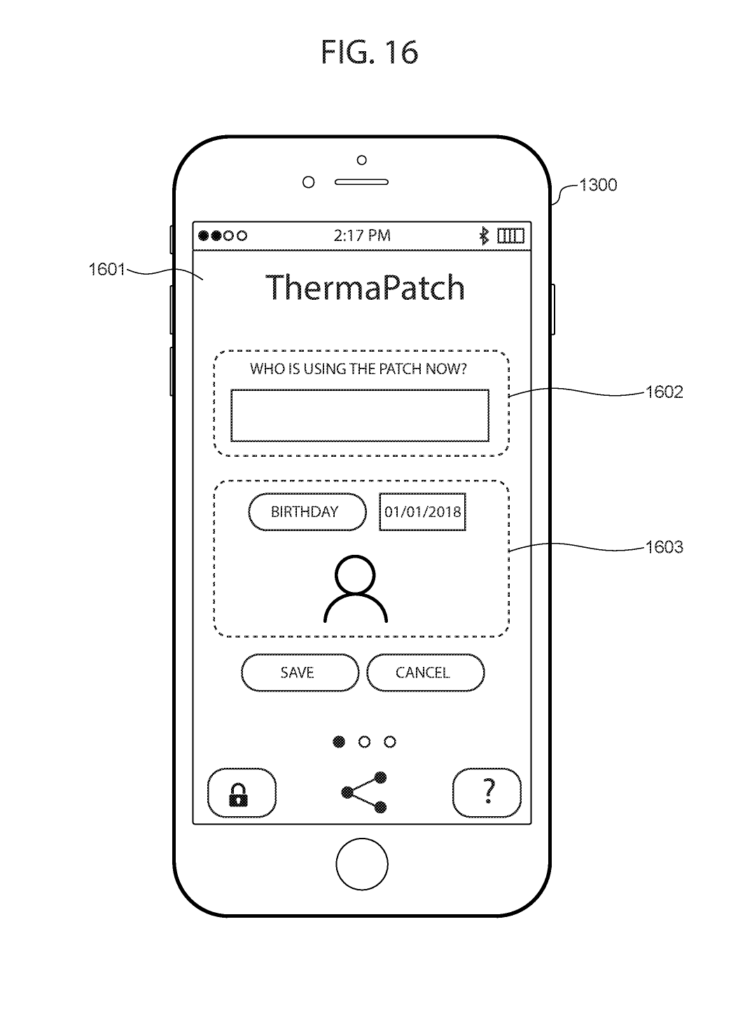

As such, in FIG. 16, a display output 1601 may be provided by the application, wherein a user identification prompt 1602 and a user information request prompt 603 may be displayed, requesting the user to provide said requested information. This may be achieved in a typical fashion, without deviating from the scope of the present invention.

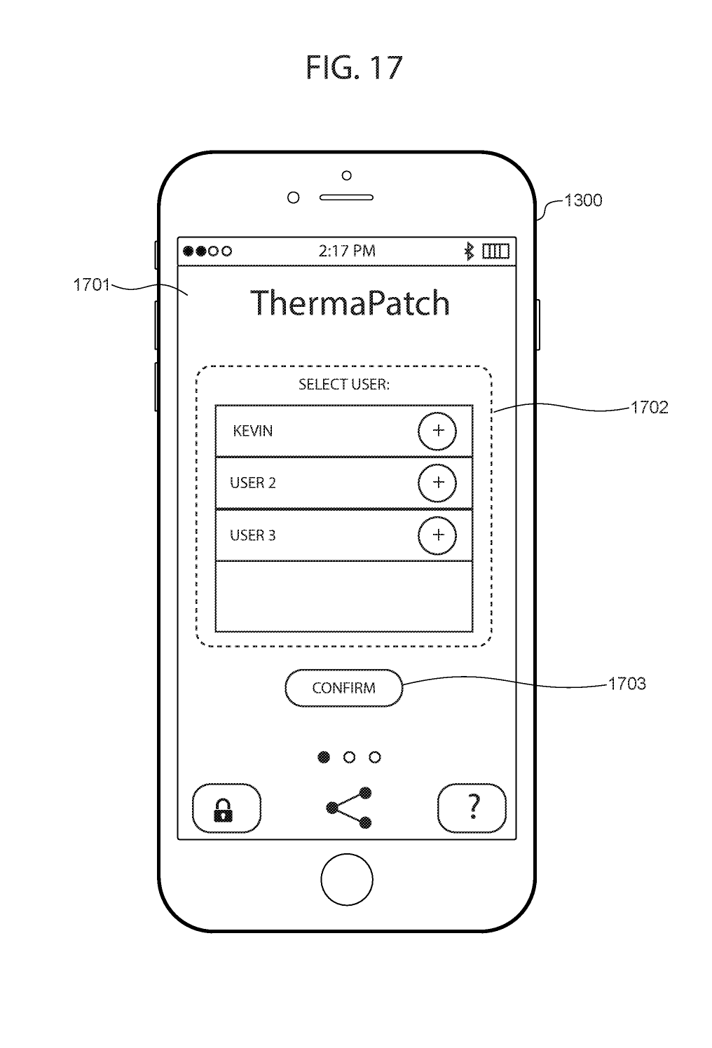

Upon saving a particular user, a display output 1701 as exemplarily shown in FIG. 17 may be provided, in which a list display 1702 of previously entered users may be generated for the user in order to enable the user to select the desired user they will be associating with the new wearable device. A typical confirmation or save button 1703 may be provided to save this user request.

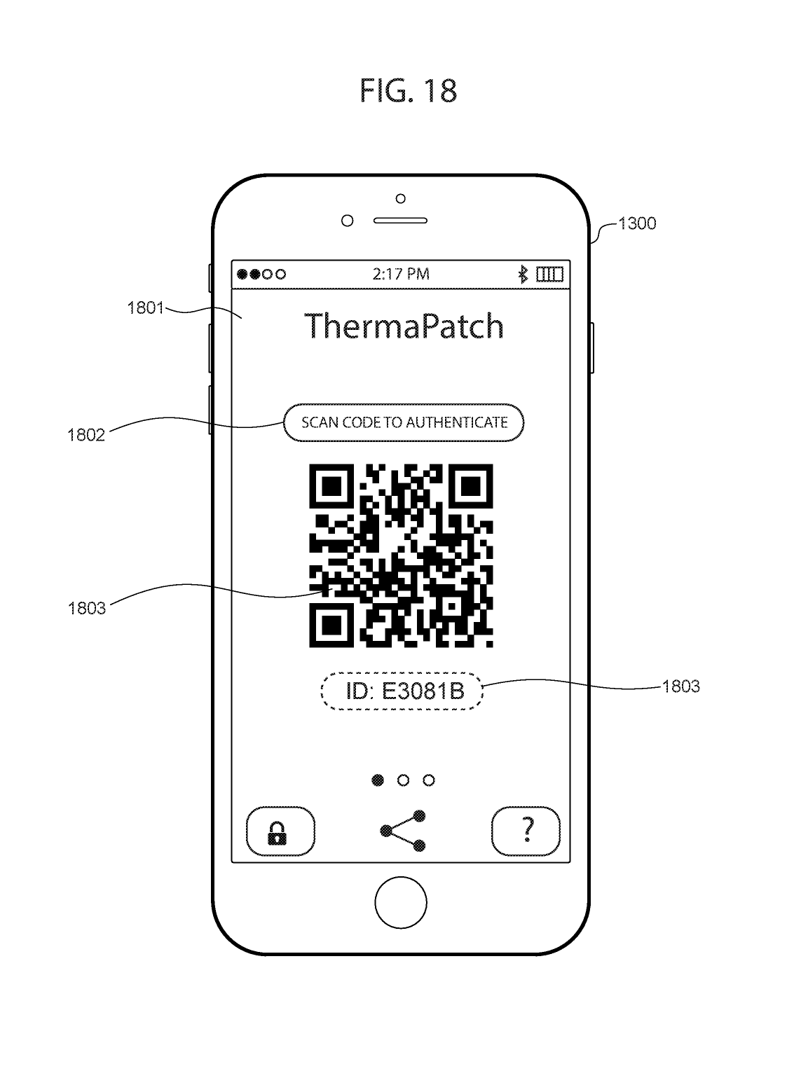

As shown in FIG. 18, once a user has been selected, a display output 1801 may prompt the user to verify, register and or authenticate the wearable device, and as such associated it to the selected user. Accordingly display output 1801 may include an authorization functionality such as a two-dimensional barcode matrix reader—for example a QR™ code reader, requesting or prompting the user to read such identification code on the wearable device or a packaging or literature of the wearable device. Accordingly, in exemplary embodiments, device 1300 may be utilized to read code 1803 upon request 1802 to read the same and display the associated ID 1803 of the wearable device that was initially displayed via indicator 1502.

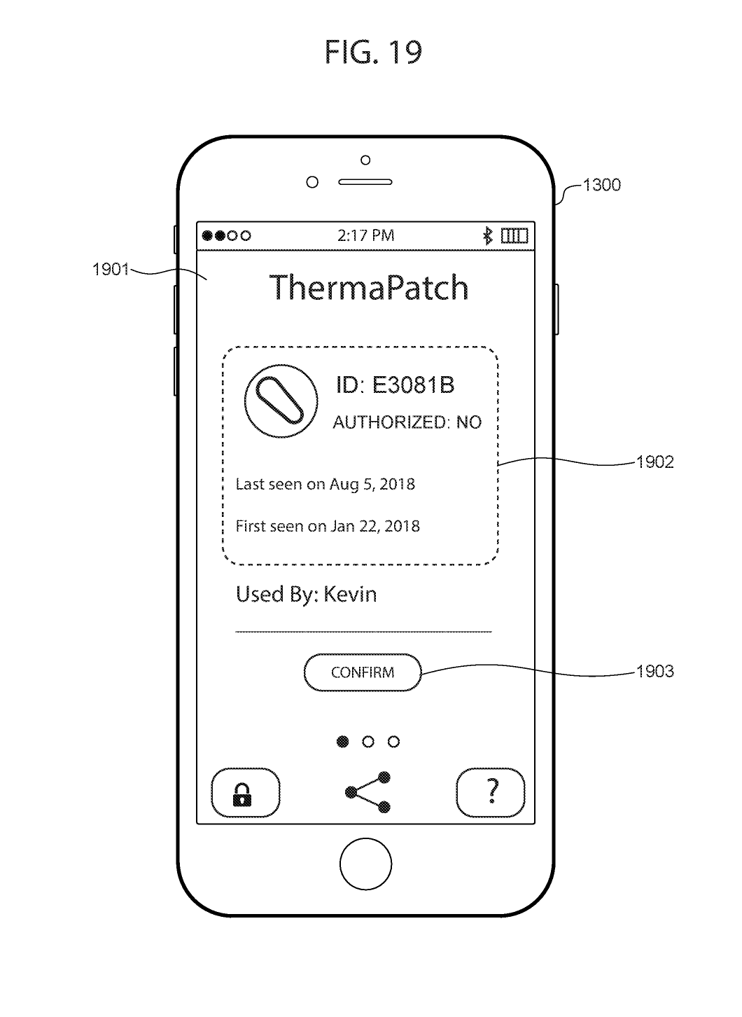

As shown in FIG. 19, a following display output 1901 may then be provided including information display 1902, wherein a user may confirm and thus associate the selected user with the registered wearable device via a typical confirmation button 1903.

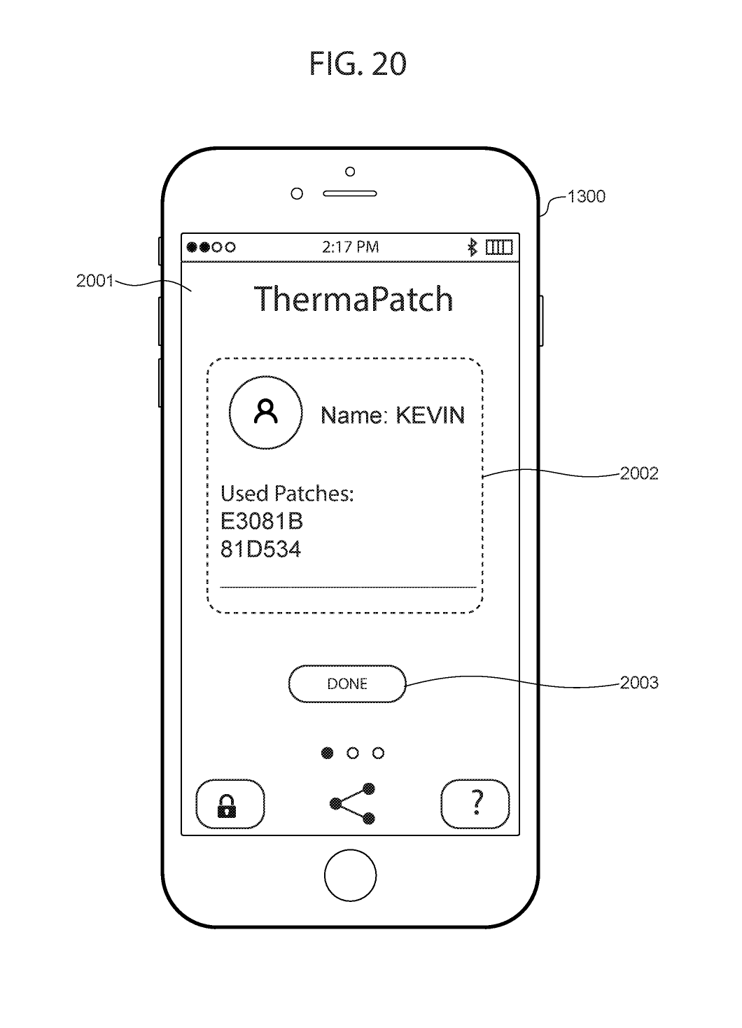

As a person of ordinary skill in the art will appreciate, a typical following screen 2001 may be provided as in FIG. 20, in which a summary of the registered device and associated user information 2002 is displayed with a simple completion button 2003. From a backend perspective, this process, although seamless to the user, may be as complex as the security protocols discussed above, or as simple as a pairing of the device to the client device 1300, without deviating from the scope of the present invention. Typically, however, various levels of encryption are implemented to make sure that no other device received the health data—whether temperature data or voltage differentials, or otherwise, sent between wearable device and client device.

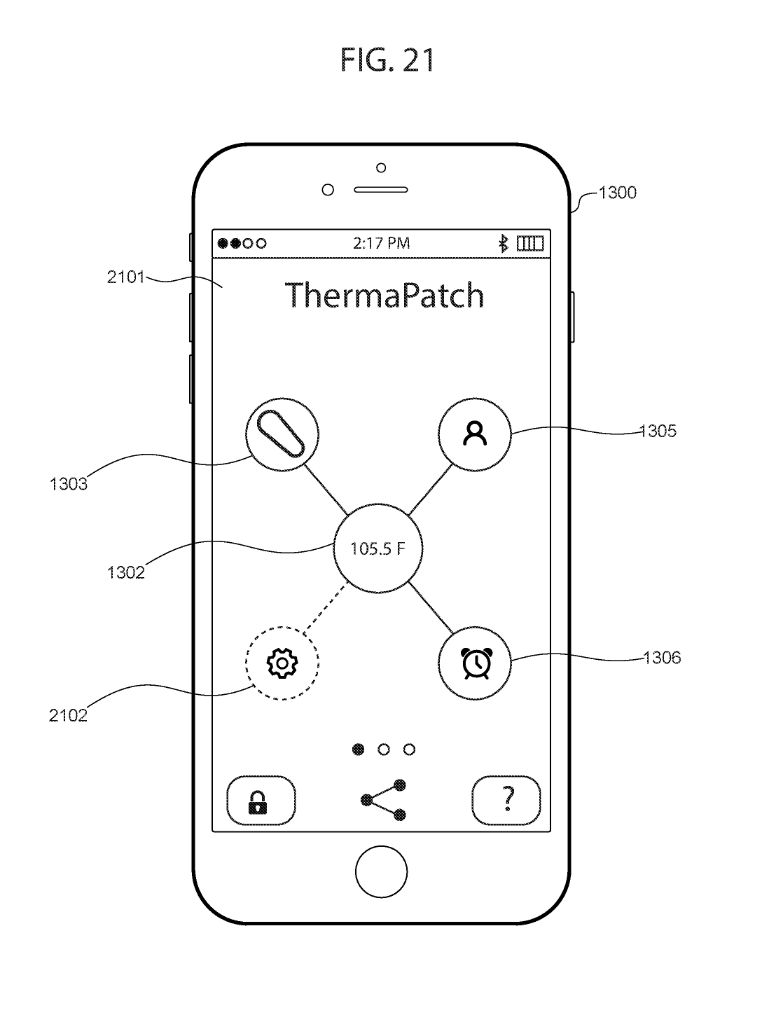

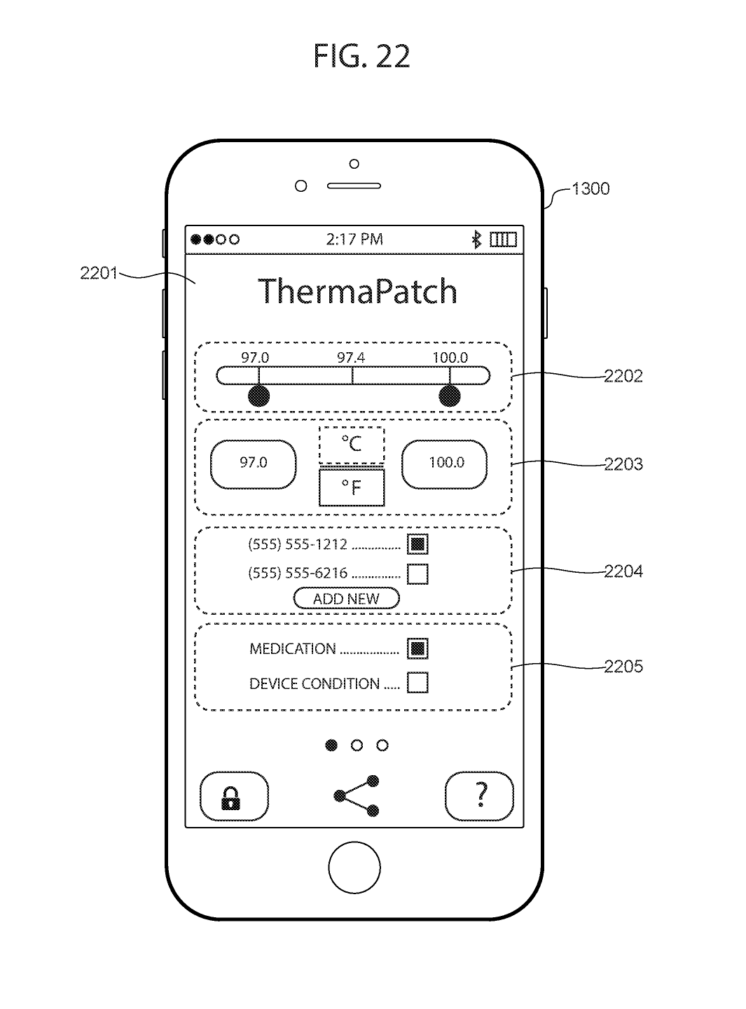

Now with reference to FIG. 21 and FIG. 22, a user may be exemplarily guided to their settings functionalities. For example, a display output 2101 may include an indicator 2102 that is blinking, lit up, the only activated or enabled function or otherwise interactable object data to guide the user to select the same. Selecting indicator 2102 may provide the user with display output 2201 in FIG. 22, in which various settings and options are provided to the user, including but not limited to: a temperature range indicator 2202 for setting off reminders or notifications; a temperature units selector 2203 for selecting the preferred temperature units to display; a contact number list 2204 for sending notifications or alerts to other client devices (such as a physicians client device, a parent's smartphone or otherwise, and or adding additional contact client devices; and or a selectable list 2205 of reminders, including for example reminders associated with a medication time, or a device condition reminder to alert the user of times for taking a medication or alerts associated with a low device battery. With regards to the latter, while the settings display may provide a user to select the types of alarms, notifications and reminders, another display output may be dedicated to control the same.

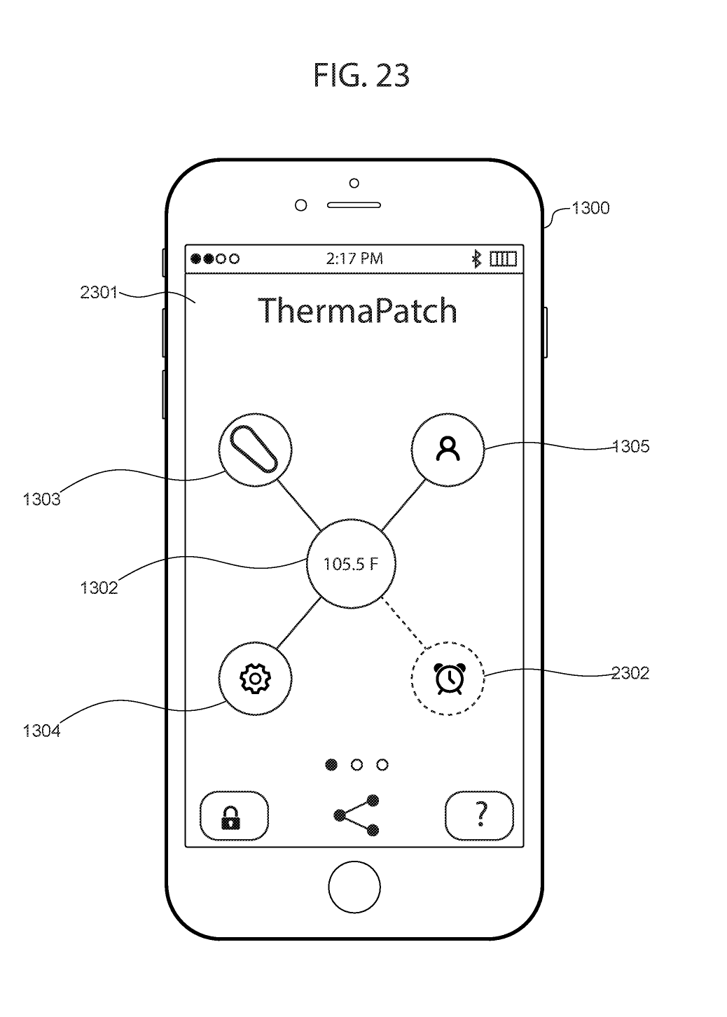

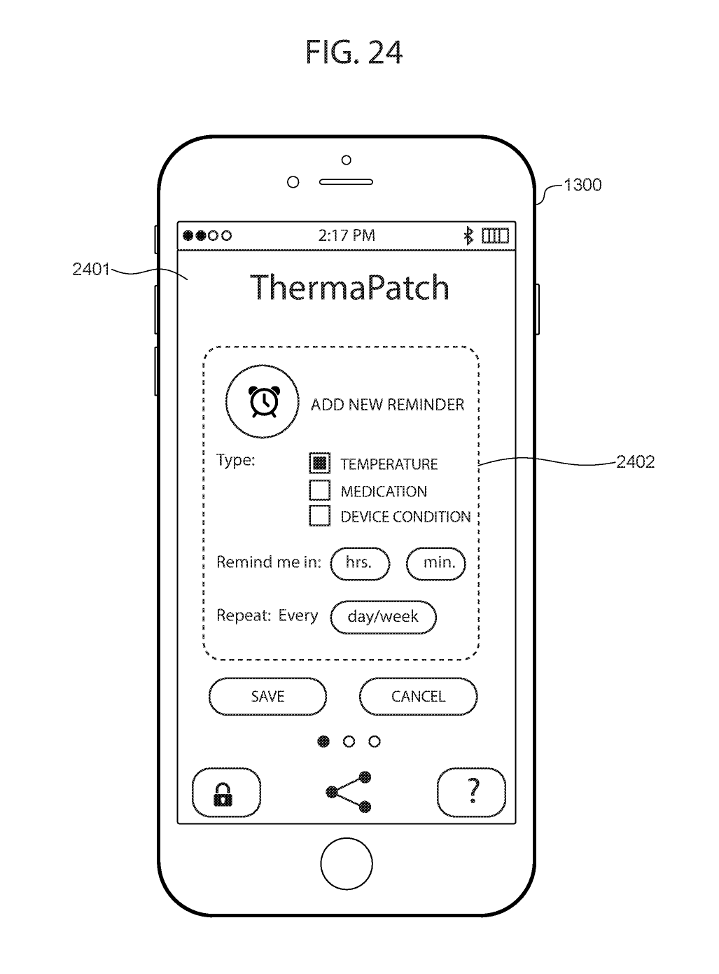

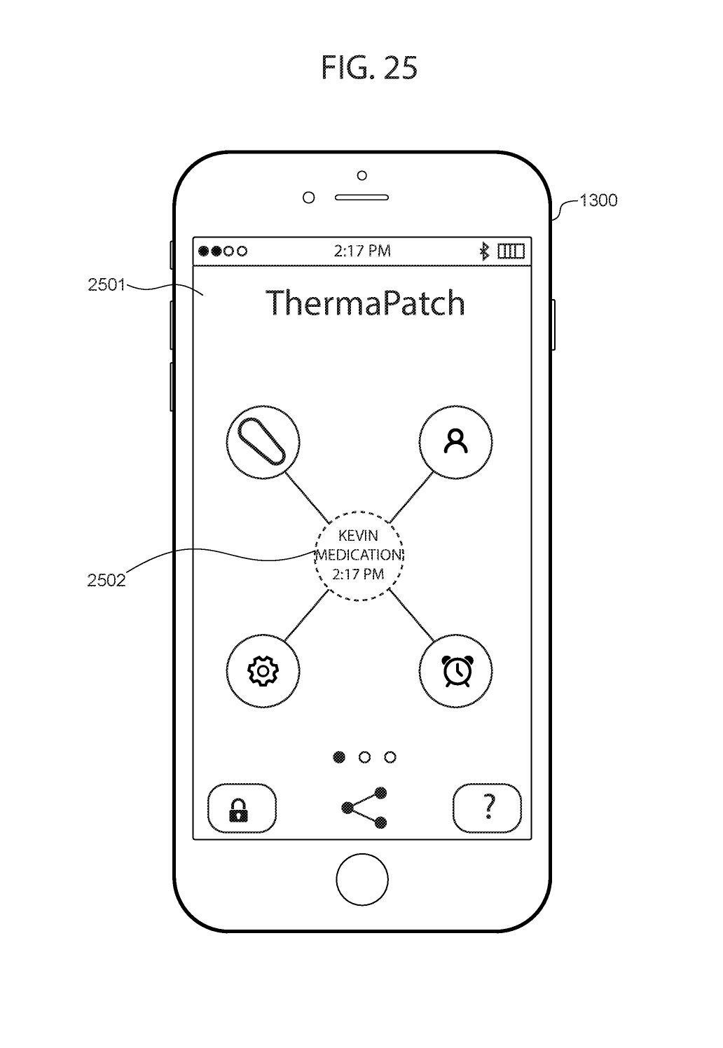

With reference to FIG. 23-FIG. 25, a display output 2300 may include an indicator 2302 to guide the user to control, add or delete a particular type of reminder, alarm, notification or otherwise. For example, and without limiting the scope of the present invention, upon selecting indicator 2302, may provide the user with display output 2401 in FIG. 24, in which a typical reminder functionality may be provided via a reminder prompt 2402. As a person of ordinary skill in the art may appreciate, prompt 2402 may include adding a type of reminder (for example whether for medication, temperature reading or device condition), as well as a time and date and frequency of the reminder, alert or notification. FIG. 25, for example, may be a display output 2501, which includes an indicator 2502 that alerts the user to a particular reminder—such as taking a medication at a previously scheduled time. Of course, other types of notifications may be incorporated via the exemplary application, including typical notifications outside of the application, including text messages, emails, audio alarms and the like.

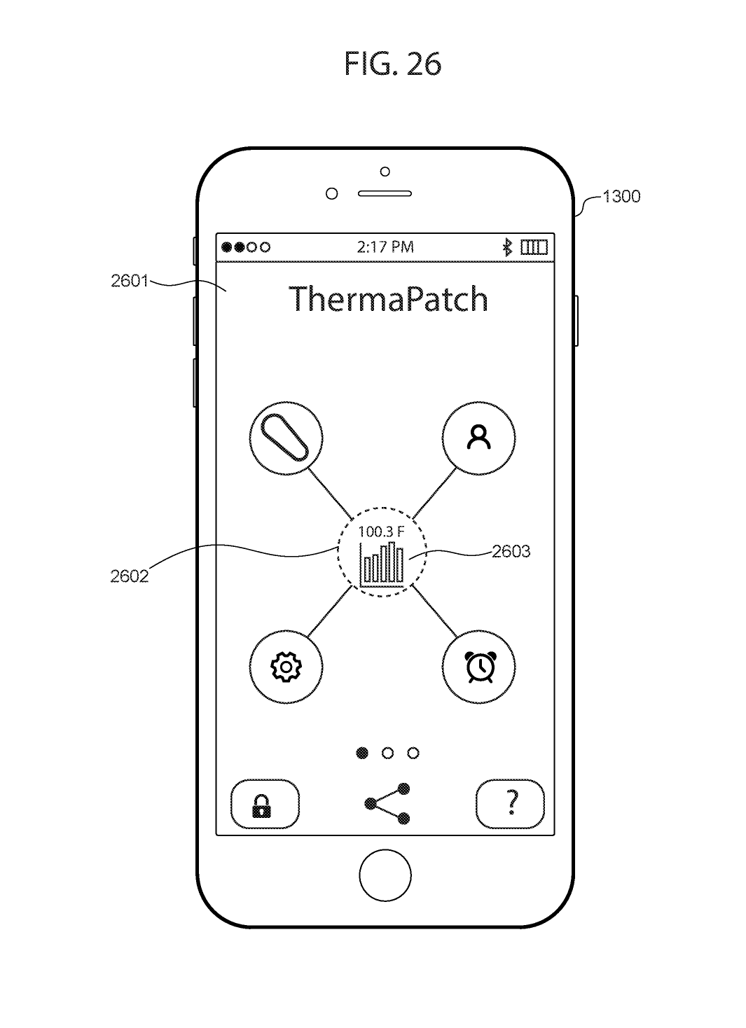

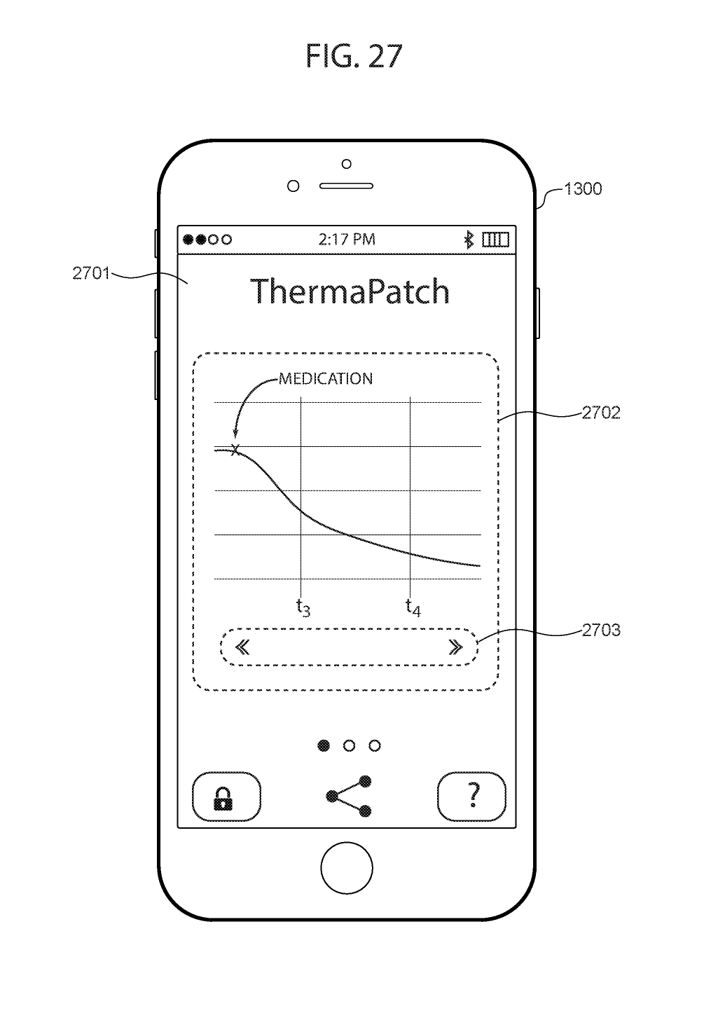

Now with reference to FIG. 26 and FIG. 27, examples of display outputs associated with tracked temperature data are provided. For example, display output 2601 may include a simple and concise display of a temperature reading via an indicator 2602, that in addition to a temperature measurement includes statistical data in a simplified graphical format 2603. In some exemplary embodiments, because more information may be available to a user, such as statistical information associated with the temperature data displayed via indicator 2602, indicator 2602 may be blinking or otherwise prompting the user as mentioned above. As such, upon interacting with indicator 2602, a new display output 2701 on FIG. 27 may be provided, wherein a graphical representation such as the one discussed with reference to FIG. 7(c) may be provided via client device 1300. Moreover, other features such as navigational functionalities 2703 may be provided to allow the user to gain valuable insights into statistics such as how well a medication is working, or whether the medication is working at all.

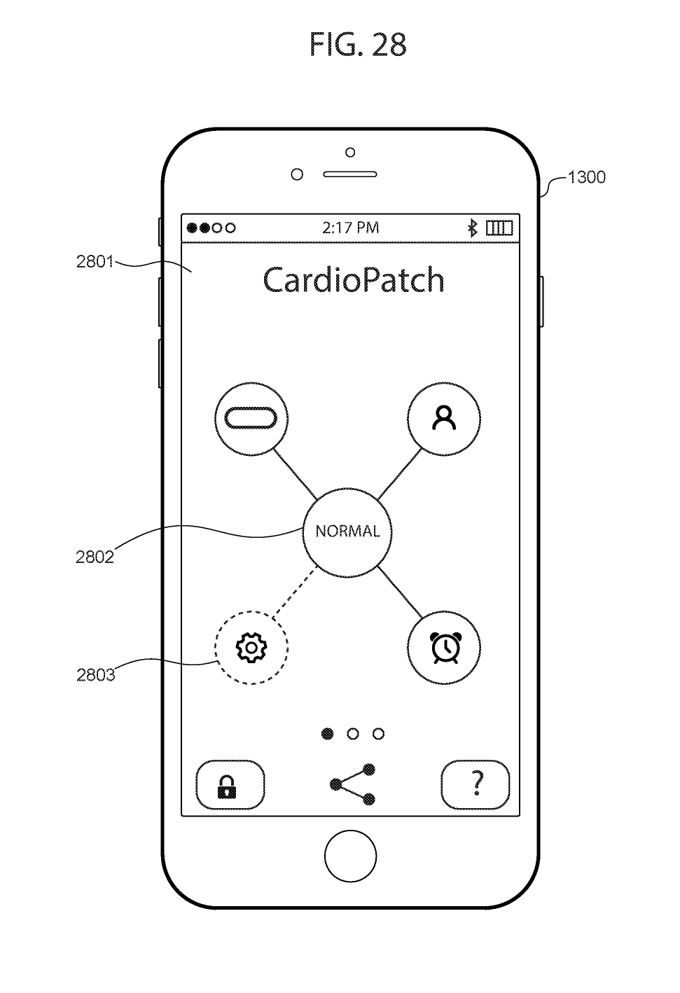

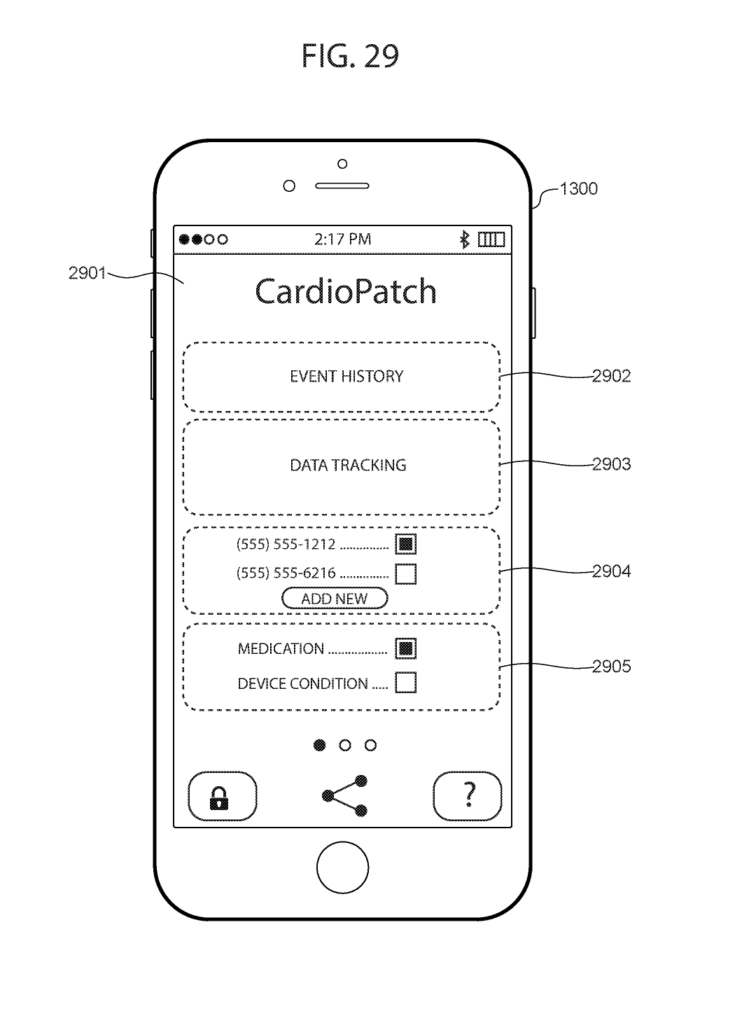

As mentioned above, such a mobile application may include functionalities for a wearable device in accordance with a device such as that disclosed with reference to FIG. 5(a)-(e). Accordingly, FIG. 28 and FIG. 29 display exemplary display outputs 2801 and 2901 that may be provided to display data that may be captured by said device. For example, and without deviating from the scope of the present invention, display output 2801 may include an alert indicator 2802 that indicates a normal voltage differential data from the user. In some exemplary embodiments, selecting indicator 2803 may provide a user with several settings, such as: an event history selection prompt 2902; a data tracking prompt 2903 for providing data tracking options; a contact number list 2904 for sending notifications or alerts to other client devices (such as a physician's client device, a parent's smartphone or otherwise, and or adding additional contact client devices; and or a selectable list 2905 of reminders, including for example reminders associated with a medication time, or a device condition reminder to alert the user of times for taking a medication or alerts associated with a low device battery.

It is an objective of the present invention to address the problems raised above and to further improve the quality of the experience provided to the user. It is another objective of the present invention to minimize latency in accurately measuring body core temperature of a user, so that information may be gleaned and acted upon quickly as needed. It is another objective of the present invention to provide reliable communication between the wearable device and client devices. It is yet another objective of the present invention to increase efficiency and generally prolong battery life. It is another objective of the present invention to allow multiple trusted client devices to access user data when they come in range of the wearable device. It is yet another objective of the present invention to protect user data from unauthorized access by storing and transmitting data securely. It is yet another objective of the present invention to generate insights from data collected. It is yet another objective of the present invention to provide alerts and notifications to various entities depending on the nature of the alert and status reports desired by the entity. These advantages and features of the present invention are not meant as limiting objectives, but are described herein with specificity so as to make the present invention understandable to one of ordinary skill in the art

A system and method of continuous health monitoring has been described. The foregoing description of the various exemplary embodiments of the invention has been presented for the purposes of illustration and disclosure. It is not intended to be exhaustive or to limit the invention to the precise form disclosed. Many modifications and variations are possible in light of the above teaching without departing from the spirit of the invention.

What is claimed is:

1. A wearable device, comprising:
a flexible printed circuit board (PCB) that is folded to form multiple layers;
a temperature sensor situated on a first surface of a first layer of the PCB;
a circuit including a thermal contact region etched on a second surface of the first layer of the PCB, the circuit further including one or more heat pathways connecting the thermal contact region of the circuit to the temperature sensor;
a communication transmitter including an antenna situated on one of the multiple layers of the PCB; and
a microprocessor in communication with the temperature sensor and the communication transmitter, the microprocessor configured to continuously obtain temperature sensing data from the temperature sensor and transmit the sensing data to one or more client devices.

2. The wearable device of claim 1, further comprising
a fold in the PCB adapted to receive a battery for powering the PCB, the fold connecting the circuit of the PCB to a cathode and to an anode of the battery.

3. The wearable device of claim 1, wherein the one or more heat pathways comprise of a vertical interconnect structure (VIA) between the first layer and the second layer of the PCB, the VIA configured to conduct heat from the thermal contact region to the temperature sensor.

4. The wearable device of claim 3, wherein the microprocessor is further configured to use a residual battery capacity to determine a transmitter payload.

5. The wearable device of claim 3, wherein the microprocessor is further configured to throttle a payload of sensing data, by:
computing a rate of change of the temperature data;
determining a state of the wearable device;
estimating a residual battery capacity; and
determining a payload format including a duration of a time interval to a next transmission in order to throttle the transmitted sensing data to the one or more client devices.

6. The wearable device of claim 3, wherein the microprocessor or at least one of the one or more client devices is further configured to compute a basal temperature of the wearer by:
receiving time-stamped sensing data including body temperature and a state of the wearable device;
implementing a buffer range of temperature data; and
determining the basal body temperature by ignoring body temperature data within the buffer range.

7. The wearable device of claim 3, further comprising a housing adapted to secure the PCB, the housing including:
a first plastic layer adapted to receive the PCB; and
a second plastic layer including an aperture for exposing the thermal region of the PCB.

8. The wearable device of claim 7, wherein the second plastic layer further comprises a thin layer over the aperture between the thermal contact region and the exterior of the housing.

9. The wearable device of claim 7, wherein the second plastic layer comprises a middle layer adapted to threadedly secure the PCB therein, and further comprising a third plastic layer for sandwiching the PCB threadedly secured in the middle layer between the first and third plastic layers.

10. The wearable device of claim 3, further comprising a heat guide, including:
a thermal insulation applied on top of the temperature sensor and the thermal contact region, the heat guide for drawing heat energy from a body of a user to the temperature sensor and minimizing a heat loss.

11. A system for continuous health monitoring, comprising:
a server for storing health data including temperature data;
one or more client devices configured to display information associated with the temperature sensing data; and
a wearable device including:
a flexible printed circuit board (PCB) that is folded to form multiple layers;
a temperature sensor situated on a first surface of a first layer of the PCB;
a circuit including a thermal contact region etched on a second surface of the first layer of the PCB, the circuit further including one or more heat pathways connecting the thermal contact region of the circuit to the temperature sensor;
a communication transmitter including an antenna situated on a one of the multiple layers of the PCB; and
a microprocessor in communication with the temperature sensor and the communication transmitter, the microprocessor configured to continuously obtain temperature sensing data from the temperature sensor and transmit the sensing data to the one or more client devices.

12. The system of claim 11, further comprising
a fold in the PCB adapted to receive a battery for powering the PCB, the fold connecting the circuit of the PCB to a cathode and to an anode of the battery.

13. The system of claim 11, wherein the one or more heat pathways comprise of a vertical interconnect structure (VIA) between the first layer and the second layer of the PCB, the VIA configured to conduct heat from the thermal contact region to the temperature sensor.

14. The system of claim 11, wherein the microprocessor is further configured to use a residual battery capacity to determine a transmitter payload.

15. The system of claim 11, wherein the microprocessor or at least one of the one or more client devices is further configured to throttle a payload of sensing data, by:
computing a rate of change of the temperature data;
determining a state of the wearable device;
estimating a residual battery capacity; and
determining a payload format including a duration of a time interval to a next transmission in order to throttle the transmitted sensing data to the one or more client devices.

16. The system of claim 11, wherein the microprocessor or at least one of the one or more client devices is further configured to compute a basal temperature of the wearer by:
receiving time-stamped sensing data including body temperature and a state of the wearable device;
implementing a buffer range of temperature data; and
determining the basal body temperature by ignoring body temperature data within the buffer range.

17. A method for continuous health monitoring, implemented by a wearable device and an executable graphical user interface (GUI) distributed to one or more client devices in communication with the wearable device, comprising:
receiving temperature data from one or more sensors on a flexible printed circuit board (PCB) of the wearable device that is folded to form multiple layers, wherein at least one of the one or more temperature sensors are situated on a first surface of a first layer of the PCB, the PCB including a circuit comprising a thermal contact region etched on a second surface of the first layer of the PCB and one or more pathways connecting the thermal contact region of the circuit to the temperature sensor;
generating one or more data packets associated with the temperature data; and
sending via a communication transmitter including an antenna situated on one of the multiple layers of the PCB, the one or more data packets associated with the temperature data to a client device.

18. The method of claim 17, further comprising:
receiving, by the client device, the temperature data from the wearable device worn by a user, the temperature data including an instantaneous temperature of the user;
computing, by the client device, a basal body temperature of the user for a predetermined period; and
deriving, from the basal body temperature of the user, an estimated fertility period for the user.

19. The method of claim 18, further comprising:
prior to computing the basal body temperature, detecting an elevation of the body temperature; and
compensating for the elevation of the body temperature.

20. The method of claim 18, further comprising:
launching, by the GUI in response to receiving the temperature data, an initial screen displaying a current temperature reading of a wearer of the wearable device; and
prompting a user of the GUI to tag the temperature reading by providing a limited set of actionable data objects on the screen display or by guidance from a blinking data object.
